(19) United States
(12) Patent Application Publication
(10) Pub. No.: US 2023/0159598 A1
(43) Pub. Date: May 25, 2023

(54) POLYPEPTIDE CLEAVAGE METHODS

(71) Applicant: ABSCI Corporation, Vancouver, WA (US)

(72) Inventors: Tyler Nusca, Portland, OR (US); Johan Kers, Vancouver, WA (US); Matthew Weinstock, Vancouver, WA (US); Michelle Slind, Vancouver, WA (US); Amy Doerner, Vancouver, WA (US); Michael Cammarata, Vancouver, WA (US); Abigail Ward, Vancouver, WA (US)

(21) Appl. No.: 17/919,717

(22) PCT Filed: Apr. 20, 2021
(86) PCT No.: PCT/US21/28249

§ 371 Date: Oct. 18, 2022

Related U.S. Application Data
(60) Provisional application No. 63/012,789, filed on Apr. 20, 2020.

(51) Int. Cl. C07K 14/195; C12N 15/62; C12N 15/70; C07K 16/32

(52) U.S. Cl. CPC C07K 14/195; C12N 15/62; C12N 15/70; C07K 16/32; C07K 2319/21; C07K 2319/92; C07K 2319/50; C07K 2319/30; C07K 2317/24; C07K 2317/14; C07K 2317/55

(57) ABSTRACT

Methods and constructs for the cleavage of polypeptides at one or more specific positions within the polypeptide are provided.

### CROSS REFERENCE TO RELATED APPLICATIONS

This application claims the benefit of U.S. Provisional Application No. 63/012,789, filed Apr. 20, 2020, which is incorporated herein by reference in its entirety.

### FIELD

The present invention is in the general technical fields of molecular biology and biotechnological manufacturing. More particularly, the present invention is in the technical field of biotechnological methods for the cleavage of polypeptides and proteins at specific locations.

### BACKGROUND

Biotechnological manufacturing (or ‘biomanufacturing’) of biological materials, such as therapeutic proteins, immunogenic compounds, or industrial enzymes and biomaterials, is a complex process. Many biological materials are manufactured as intermediates, for example, as polypeptides that include tags or other sequences useful in manufacturing and/or purification, that are further processed to form the final biological product. One biomanufacturing challenge is the removal of amino acid residues from the N-terminus of polypeptides, including the N-terminal methionine residue—which can be a modified ‘fMet’ (N-formylmethionine) in the case of prokaryotic host cells—to generate proteins having the ‘native’ N-terminal amino acid residue found on mature forms of that protein. The need to develop methods for the removal of N-terminal amino acid residues is particularly acute for expression systems that are designed to produce proteins in the cytoplasm of host cells, since there will be no cleavage of signal peptides from such proteins during translocation across a membrane.

Another challenge in biomanufacturing is the production of polypeptides and proteins that are toxic to the host cell used in their production. Improved protein expression methods, capable of cleaving polypeptides and proteins at specific residues and useful in the production of toxic proteins, are clearly needed.

### SUMMARY

The present disclosure provides intein polypeptides and methods using intein polypeptides for the cleavage of polypeptides and proteins at specific residues. Provided herein are intein polypeptides, polynucleotides encoding intein polypeptides, expression constructs, assay methods and host cells, which can be combined in various embodiments.

In some embodiments, provided herein are intein polypeptides comprising an amino acid sequence having at least 70% amino acid sequence identity (e.g., at least 70%, at least 80%, at least 90%, or at least 95% amino acid sequence identity) to an amino acid sequence selected from the group consisting of amino acids 105-124 of SEQ ID NO:2, amino acids 93-112 of SEQ ID NO:1, amino acids 85-104 of SEQ ID NO:4, amino acids 1-21 of SEQ ID NO:12, and amino acids 1-21 of SEQ ID NO:15. In other embodiments, provided herein are intein polypeptides comprising an amino acid sequence having at least 60% amino acid sequence identity (such as at least 60%, at least 70%, at least 80%, or at least 90% amino acid sequence identity) to an amino acid sequence selected from the group consisting of amino acids 100-129 of SEQ ID NO:2, amino acids 88-117 of SEQ ID NO:1, amino acids 80-109 of SEQ ID NO:4, amino acids 1-30 of SEQ ID NO:12, and amino acids 1-30 of SEQ ID NO:15. In some examples, the intein polypeptide comprises an amino acid sequence selected from the group consisting of amino acids 105-124 of SEQ ID NO:2, amino acids 93-112 of SEQ ID NO:1, amino acids 85-104 of SEQ ID NO:4, amino acids 1-21 of SEQ ID NO:12, and amino acids 1-21 of SEQ ID NO:15; or comprises an amino acid sequence selected from the group consisting of amino acids 88-117 of SEQ ID NO:1, amino acids 100-129 of SEQ ID NO:2, amino acids 80-109 of SEQ ID NO:4, amino acids 1-30 of SEQ ID NO:12, and amino acids 1-30 of SEQ ID NO:15. In other embodiments, provided herein are intein polypeptides comprising an amino acid sequence having at least 70% amino acid sequence identity (e.g., at least 70%, at least 80%, at least 90%, or at least 95% amino acid sequence identity) to the amino acid sequence of amino acids 105-124 of any one of SEQ ID NOs:120-124 or comprising an amino acid sequence having at least 60% amino acid sequence identity (such as at least 60%, at least 70%, at least 80%, or at least 90% amino acid sequence identity) to the amino acid sequence of amino acids 100-129 of any one of SEQ ID NOs:120-124.

In some examples, the intein polypeptide lacks an N-terminal cysteine residue and/or lacks substantial extein-ligating activity. In additional examples, the intein polypeptide includes a polyhistidine tag (e.g., a 6×His tag) and/or includes a cleavage sequence cleavable by a protease. In particular non-limiting examples, the intein polypeptide comprises an amino acid sequence having at least 70% amino acid sequence identity (e.g., at least 70%, at least 80%, at least 90%, or at least 95% amino acid sequence identity) to the amino acid sequence of any one of SEQ ID NOs: 1, 2, 4, 12, 15. In other non-limiting examples, the intein polypeptide comprises an amino acid sequence having at least 70% amino acid sequence identity (e.g., at least 70%, at least 80%, at least 90%, or at least 95% amino acid sequence identity) to the amino acid sequence of any one of SEQ ID NOs: 120-124.

Also provided herein are fusion polypeptides including the amino acid sequence of a disclosed intein polypeptide and the amino acid sequence of a target polypeptide. In some embodiments, the N-terminal amino acid of the amino acid sequence of the target polypeptide is the N-terminal amino acid of the amino acid sequence of a mature form of the target protein. In other embodiments, the target polypeptide can form one or more disulfide bonds. In some non-limiting examples, the target polypeptide is selected from the group consisting of: an antibody heavy chain, an antibody light chain, and fragments thereof. In additional examples, the intein fusion polypeptide lacks a signal sequence. In other examples, the intein amino acid sequence lack an N-terminal cysteine residue and/or lacks substantial extein-ligating activity. In additional examples, the intein fusion polypeptide includes a polyhistidine tag (e.g., a 6×His tag) and/or includes a cleavage sequence cleavable by a protease.

Also provided are polynucleotides encoding the disclosed intein polypeptides. In particular embodiments, the polynucleotide encoding the intein polypeptide comprises a nucleotide sequence selected from the group consisting of SEQ ID NOs 24-119.

Disclosed herein are expression constructs comprising two or more intein-encoding polynucleotide sequences, wherein each intein-encoding polynucleotide sequence differs from every other intein-encoding polynucleotide sequence, In some embodiments, every intein polypeptide encoded by the intein-encoding polynucleotide sequences has the same amino acid sequence. In other embodiments, at least two of the intein polypeptides encoded by the intein-encoding polynucleotide sequences have different amino acid sequences. In some embodiments, the two or more intein-encoding polynucleotide sequences are selected from the group consisting of SEQ ID NOs: 24-119.

In some examples, the intein-encoding polynucleotide sequences, and the amino acid sequence of the intein polypeptide encoded by those intein-encoding polynucleotide sequences, are selected from the group consisting of: (a) polynucleotide sequences SEQ ID NOs 26 and 27, and amino acid sequence SEQ ID NO:2; (b) polynucleotide sequences SEQ ID NOs 24 and 25, and amino acid sequence SEQ ID NO:1; (c) polynucleotide sequences SEQ ID NOs 28 and 29, and amino acid sequence SEQ ID NO:3; (d) polynucleotide sequences SEQ ID NOs 30 and 31, and amino acid sequence SEQ ID NO:4; (e) polynucleotide sequences SEQ ID NOs 32 and 33, and amino acid sequence SEQ ID NO:5; (f) polynucleotide sequences SEQ ID NOs 34 and 35, and amino acid sequence SEQ ID NO:6; (g) polynucleotide sequences SEQ ID NOs 36 and 37, and amino acid sequence SEQ ID NO:7; (h) polynucleotide sequences SEQ ID NOs 38 and 39, and amino acid sequence SEQ ID NO:8; (i) polynucleotide sequences SEQ ID NOs 40 and 41, and amino acid sequence SEQ ID NO:9; (j) polynucleotide sequences SEQ ID NOs 43 and 44, and amino acid sequence SEQ ID NO:11; (k) polynucleotide sequences SEQ ID NOs 45 and 46, and amino acid sequence SEQ ID NO:12; (l) polynucleotide sequences SEQ ID NOs 48 and 49, and amino acid sequence SEQ ID NO:14; (m) polynucleotide sequences SEQ ID NOs 50 and 51, and amino acid sequence SEQ ID NO:15; (n) polynucleotide sequences SEQ ID NOs 53 and 54, and amino acid sequence SEQ ID NO:17; (o) polynucleotide sequences SEQ ID NOs 56 and 57, and amino acid sequence SEQ ID NO:19; (p) polynucleotide sequences SEQ ID NOs 59 and 60, and amino acid sequence SEQ ID NO:21; and (q) any two or more of polynucleotide sequences SEQ ID NOs 26, 27, 61-66, and 79-119, and amino acid sequence SEQ ID NO:2. In some examples, the expression construct is an expression vector. In additional examples, the expression construct is a dual-promoter expression vector, for example, a dual-promoter expression vector comprising an L-arabinose-inducible promoter and a propionate-inducible promoter.

Methods for producing a target polypeptide are also provided. In some embodiments, the methods include generating a composition comprising an intein fusion polypeptide provided herein, wherein the intein amino acid sequence self-excises from the intein fusion polypeptide, thereby producing the target polypeptide; and recovering the target polypeptide from the composition. In some embodiments, generating the composition includes expressing the intein fusion polypeptide in a host cell. The methods may further include lysing the host cell. In some examples, lysing the host cell generates the composition. In some embodiments, the host cell has a reduced level of function of thioredoxin reductase and a reduced level of function of a protein selected from the group consisting of glutathione reductase and glutathione synthetase. For example, the host cell has an altered form of the gene encoding AhpC selected from the group consisting of the ahpC*, ahpC^{Δ}, V164G, S71F, E173/S71F, E171Ter, and dup162-169 mutations and/or the host cell comprises a polynucleotide encoding a cytoplasmic form of DsbC. In some embodiments, the host cell is a prokaryotic cell, such as an Escherichia coli cell (for example, Escherichia coli B strain 521 cell).

Also provided are host cells comprising the disclosed expression constructs. In some embodiments, the host cell has a reduced level of function of thioredoxin reductase and a reduced level of function of a protein selected from the group consisting of glutathione reductase and glutathione synthetase. In some examples, the host cell has an altered form of the gene encoding AhpC selected from the group consisting of the ahpC*, ahpC^{Δ}, V164G, S71F, E173/S71F, E171Ter, and dup162-169 mutations and/or the host cell comprises a polynucleotide encoding a cytoplasmic form of DsbC. In some embodiments, the host cell is a prokaryotic cell, such as an Escherichia coli cell (for example, Escherichia coli B strain 521 cell).

### BRIEF DESCRIPTION OF THE DRAWINGS

FIGS. 1A and 1B show Western blots of intein-TRAST-Fab constructs expressed in E. coli host cells. The TRAST-Fab heavy and light chains were expressed with various intein polypeptide sequences attached to each of the heavy and light chains at their N-termini; all of the inteins lacked an N-terminal cysteine residue to prevent extein ligation. The type of intein used is indicated above each blot. DnaX: Synechocystis DnaX 6×His-tagged mini-intein; DnaB: Synechocystis DnaB 6×His-tagged mini-intein; D.t. DnaB: Desulfofundulus thermosubterraneus DSM 16057 DnaB intein; gp41-1: Prochlorococcus cyanophage P-SSM2 gp41-1 6×His-tagged mini-intein; and N.p. DnaB: Nostoc punctiforme strain ATCC 29133/PCC 73102 DnaB mini-intein. ‘P’ indicates material that was pelleted by centrifugation, while ‘S’ indicates material that remained soluble. The TRAST-Fab protein samples corresponding to each well were separated by polyacrylamide gel electrophoresis under non-reducing conditions (FIG. 1A) and under reducing conditions (FIG. 1B). The Western blots made from these gels utilized a primary antibody that binds to human IgG. MW (kDa): Molecular weight (mass) markers. The positions of the uncleaved and cleaved protein bands are indicated with arrows and symbols: Panel A (non-reduced), from the top: Fab heterodimer with uncleaved intein, Fab heterodimer with intein cleaved from both polypeptides, monomeric cleaved Fab polypeptide; Panel B (reduced), from the top: monomeric Fab polypeptide with uncleaved intein, monomeric cleaved Fab polypeptide.

FIGS. 2A-2C are a series of chromatograms showing an analysis by liquid chromatography mass spectrometry (LC-MS) of the structures of intein-cleaved (but otherwise intact) intein-TRAST-Fab polypeptides in comparison to control Met-TRAST-Fab. The DnaB-TRAST-Fab and gp41-1-TRAST-Fab proteins were expressed with N-terminal intein sequences as described for FIG. 1. The protein samples were purified by affinity chromatography with Protein L, then reduced with TCEP (tris(2-carboxyethyl)phosphine) and acidified with formic acid. Panel A shows absorbance in arbitrary units of intensity plotted against elution time, and Panels B and C are in counts per second (CPS) plotted against elution time; the unit daltons (‘Da’) in the legends to Panels B and C refers to mass/charge (m/z). FIG. 2A: Absorbance of UV light at 214 nm, showing the elution from minute 13 to minute 18, which includes elution of both the light chain and the heavy chain. DnaB-TRAST-Fab exhibited peaks comparable in location to the Met-TRAST-Fab control. FIG. 2B: Extracted ion chromatogram peaks for proteins in the predicted mass/charge range for TRAST-Fab light chain with the native N-terminus. The Met-TRAST-Fab control sample does not include detectable native N-terminus light chain when viewed on the same intensity scale as the DnaB-TRAST-Fab and gp41-1-TRAST-Fab samples, which both produced appreciable amounts of native N-terminus light chain. FIG. 2C: Extracted ion chromatogram peaks for proteins in the predicted mass/charge range for TRAST-Fab heavy chain with the native N-terminus. The results for the TRAST-Fab heavy chain shown in this panel are comparable to those shown in Panel B for the light chain, but the DnaB-TRAST-Fab sample displayed higher amounts of native N-terminus heavy chain than the gp41-1-TRAST-Fab sample.

FIGS. 3A and 3B are a series of chromatograms showing an analysis by liquid chromatography mass spectrometry (LC-MS) of the structures of intein-cleaved (but otherwise intact) intein-TRAST-Fab heterodimers in comparison to control Met-TRAST-Fab heterodimer. The DnaX-TRAST-Fab, DnaB-TRAST-Fab, and D.t. DnaB-TRAST-Fab proteins were expressed with N-terminal intein sequences as described for FIG. 1. The protein samples were purified by affinity chromatography with Protein L, then acidified with formic acid, and analyzed by LC-MS under non-reducing conditions. The units for FIGS. 3A and 3B are the same as for FIGS. 2A and 2B. FIG. 3A: Absorbance of UV light at 280 nm, showing the elution from minute 11 to minute 24, which includes elution of TRAST-Fab heterodimer. The samples, particularly the DnaB-TRAST-Fab and D.t. DnaB-TRAST-Fab samples, exhibited peaks comparable in location to the Met-TRAST-Fab control. FIG. 3B: Extracted ion chromatogram peaks in the predicted mass/charge range for the TRAST-Fab heterodimer with the native N-terminus for both the heavy and the light chain. The top chromatogram shows that in the control Met-TRAST-Fab sample a detectable amount of native N-termini TRAST-Fab has been generated by N-terminal methionine removal within the host cell. However, comparison of the intensity of the peaks shows that the intein-TRAST-Fab samples have a much higher amount (between 10- and 20-fold higher) of native N-termini than the control Met-TRAST-Fab sample.

### DETAILED DESCRIPTION

The problem of cleaving polypeptides and proteins is addressed by providing the methods described herein, particularly for the purpose of producing polypeptides and proteins of interest that have a ‘native’ or otherwise desirable N-terminal amino acid residue; these are referred to herein as ‘target’ polypeptides and proteins. A native N-terminal residue is the amino acid found at the N-terminus of a fully mature protein, without considering any residues added to the protein by post-translational modification.

Intein polypeptides comprise the amino acid sequence of an intein or one or more fragments of an intein amino acid sequence; examples of intein polypeptides include mini inteins and split inteins as described further herein. Intein polypeptides are capable of self-excision from an extein polypeptide or from extein polypeptides, and may also be capable of ligating extein polypeptides to each other. Intein fusion polypeptides are intein polypeptides that comprise the amino acid sequence of an intein, or one or more fragments of an intein amino acid sequence, and also comprise the amino acid sequence of a target polypeptide.

The methods provided herein include the use of intein sequences, included within the intein fusion polypeptide sequence to be cleaved, and the self-excision of the intein sequences from the intein fusion polypeptide to generate the target polypeptide or protein. Also provided are particular intein polypeptides, and polynucleotide sequences encoding those intein polypeptides.

Inteins and the proteins that can be produced using them are described in sections I-II below. Expression constructs, host cells, and methods of producing and purifying polypeptides comprising inteins and proteins that are produced using inteins are described in sections III-V below.

### I. Inteins and Intein-Containing Expression Constructs.

Inteins and mini-inteins. Inteins are polypeptides capable of catalyzing their own excision from the N-extein and the C-extein that are adjacent to the intein sequence on its N-terminal and C-terminal side respectively. Inteins have been identified in organisms across all phylogenetic kingdoms, and for these naturally occurring inteins, the self-excision of the intein typically results in the linking of the N-extein to the C-extein through a peptide bond. The self-excision of inteins can result from interaction between intein sequences within the same polypeptide, or between intein polypeptides acting in trans (see the description of split inteins, below). Many inteins contain sequences that are not needed for the intein-excision reaction, and for the purposes of biomanufacturing, it is preferred to use ‘mini’ inteins from which any extraneous sequences have been removed (Derbyshire et al., “Genetic definition of a protein-splicing domain functional mini-inteins support structure predictions and a model for intein evolution,” Proc Natl Acad Sci USA 1997 Oct. 14; 94(21): 11466-11471; Erratum in: Proc Natl Acad Sci USA 1998 Jan. 20; 95(2): 762). Mini-inteins comprise amino acid sequences from the N-terminal end and from the C-terminal end of the intact intein, and can also comprise tags, such as 6×His polyhistidine tags, or other desirable sequences placed between the remaining N-terminal and C-terminal portions of the mini-intein.

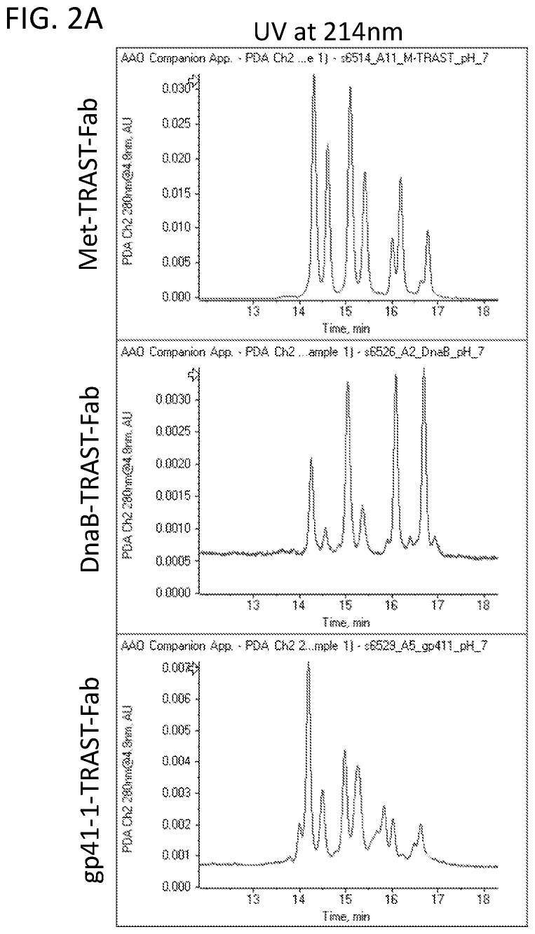

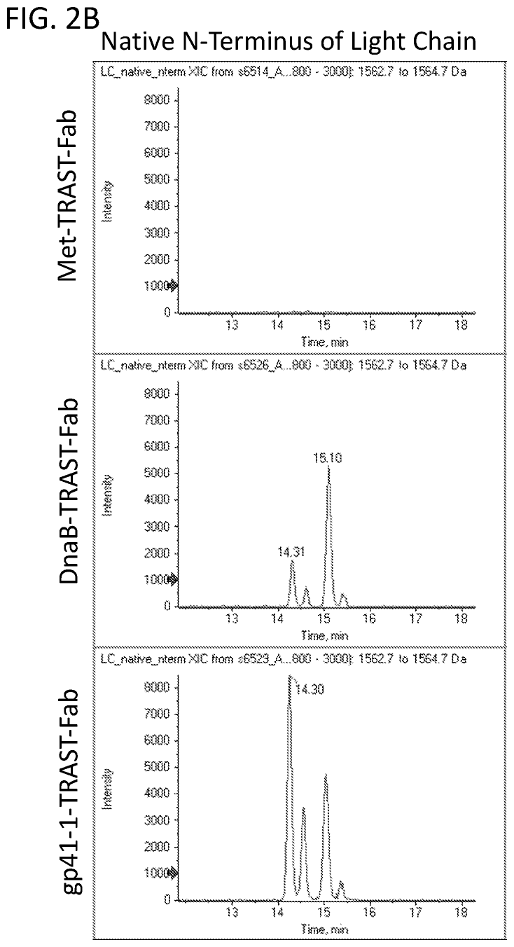

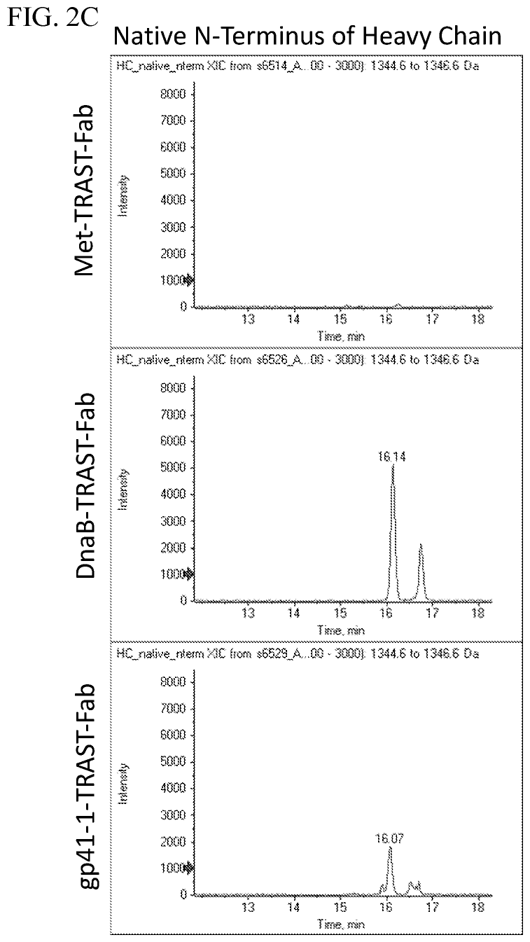

‘Non-ligating’ inteins. Alterations in the amino acid sequences of inteins have been shown to generate intein variants that are capable of self-excision from extein sequences, but which do not catalyze the linkage of the N-extein to the C-extein (Mathys et al., “Characterization of a self-splicing mini-intein and its conversion into autocatalytic N- and C-terminal cleavage elements: facile production of protein building blocks for protein ligation”, Gene 1999 Apr. 29; 231(1-2): 1-13). Specifically, when an alanine residue is substituted for the cysteine residue typically present at the N-terminal end of the intein sequence, the intein can be excised from the exteins without ligation of the exteins. Other changes to the intein amino acid sequence that alter or remove that N-terminal cysteine residue can also result in inteins that are not capable of extein ligation. Inteins, and particularly mini-inteins, that have been altered in this way are referred to herein as ‘non-ligating’ inteins. The degree to which an intein can self-excise and/or ligate exteins can be determined by expressing intein polypeptides under conditions in which excision of inteins and ligation of exteins can occur, and then quantifying the different polypeptide products that are produced, for example by quantifying different protein bands following separation of the polypeptide products by gel electrophoresis, as shown in FIG. 2 of Mathys et al. 1999, or by quantitative liquid chromatography methods. A ‘non-ligating’ intein polypeptide, which is an intein polypeptide that lacks substantial extein-ligating activity, produces less than 25% of the ligated extein product (and preferably less than 10% of the ligated extein product) when compared to the polypeptides produced by a corresponding ‘ligating’ intein polypeptide under the same conditions.

Non-ligating inteins are useful for generating any desired N-terminus of a protein produced by biotechnological methods, because they can be placed immediately upstream of the desired N-terminal amino acid residue, then cleaved off by self-excision without attaching other amino acids. For many proteins produced by biotechnological methods, and especially therapeutic proteins, the desired N-terminal residue will be the ‘native’ N-terminal amino acid found in naturally occurring forms of the protein. It is also possible to use these altered ‘non-ligating’ forms of inteins to produce proteins with desired C-terminal residues, for example when successful expression of a polypeptide requires the presence of additional residues at the C-terminal end of the polypeptide. In these cases the intein amino acid sequence immediately follows the desired C-terminal residue, and any additional amino acid sequences required for expression can be added to the center of a mini-intein, or included following the intein sequence at the C-terminal end of the polypeptide as expressed.

‘Split’ inteins are intein polypeptides in which the N-terminal and C-terminal portions of the intein are expressed as separate polypeptides that can act in trans to accomplish the excision reaction, and if the N-terminal portion of the intein has the required cysteine residue, the ligation reaction. A polypeptide comprising the C-terminal portion of the intein, for example, can also include tag polypeptides or other useful amino acid sequences in its N-terminal region, as they should not interfere with cleavage of the C-terminal intein by the N-terminal portion of the intein to produce target proteins having the desired N-terminal residue as described above. Separating the N-terminal and C-terminal portions of the intein can increase the efficiency of biotechnological production of proteins, in that the intein fusion polypeptide comprising the amino acid sequences of the target protein and of the C-terminal portion of the intein (for example) will be shorter and require less of the host cell resources per molecule to be expressed. These considerations regarding the use of the C-terminal portion of the intein in the expression of polypeptides also apply to the use of the N-terminal portion of the intein to generate a desired C-terminal residue on a protein, as described above.

Using split inteins also allows the excision (and in some cases the ligation) reaction to be spatially and/or temporally separated from the expression of polypeptides comprising the N-terminal or the C-terminal portion of the intein. For example, the portion of the intein that is to act in trans can be expressed in a different area or compartment of the host cell so that it interacts with the intein fusion polypeptide comprising the other portion of the intein following expression and movement (such as translocation) of the intein fusion polypeptide through the host cell, or it can be used ex vivo in processing of the expressed intein fusion polypeptide to produce the target mature protein. As a particular example, the N-terminal portion of a ‘non-ligating’ intein comprising a 6×His polyhistidine tag is expressed and purified, and attached to a solid support medium that comprises Ni²⁺ or Cd²⁺ metal ions. Intein fusion polypeptides comprising the amino acid sequence of a target protein and having the C-terminal portion of the intein at the N-terminal end of the intein fusion polypeptide are passed through the medium comprising the N-terminal portion of the intein, resulting in the cleavage of the C-terminal portion of the intein from the intein fusion polypeptide, releasing the target protein.

Use of inteins to produce toxic polypeptides. The ability to include additional amino acid sequences at the N- or C-terminus of an intein polypeptide in cases where ‘non-ligating’ forms of inteins can be used, or within an intein polypeptide sequence in cases where inteins capable of both excision and ligation can be used, can allow for the efficient expression of otherwise toxic polypeptides. Polypeptides of interest that have an activity that is toxic to the host cell can be rendered less toxic by the inclusion in an intein of amino acid sequences that interact with the active site(s) of the polypeptide of interest, eliminating or reducing the activity of the polypeptide and concomitantly its toxicity, until those interacting amino acid residues can be removed, for example by the intein self-excision reaction.

Intein amino acid sequences and DNA sequences encoding them. Amino acid sequences of several representative intein polypeptides are presented as SEQ ID NOs 1-21 and 120-124, as indicated in Table 1. Each of the intein polypeptides of SEQ ID NOs 1-21 and 120-124 is an altered intein of the non-ligating type and lacks an N-terminal cysteine residue. The split inteins correspond to the N-terminal and C-terminal portions of inteins and mini-inteins, as indicated in Table 1. Split inteins can optionally include additional amino acid sequences, such as the C-terminal portion of the DnaX intein or of the DnaB intein which, as presented in SEQ ID NOs 12 and 15, respectively, each comprise a 6×His tag. DNA sequences encoding these inteins are also listed in Table 1, and are described further in Example 1A.

TABLE 1
Intein Sources and Types, Amino Acid Sequences, and DNA Sequences

| Shortened | Source Species (and Strain), | Intein Amino Acid | Intein-Coding |
|---|---|---|---|
| Name | Gene; Intein Type | Sequences | DNA Sequences |
| DnaX | Synechocystis sp. PCC6803, | SEQ ID NO: 1 | SEQ ID NO: 24 |
| | DnaX; 6xHis-tagged mini-intein | | SEQ ID NO: 25 |
| DnaB | Synechocystis sp. PCC6803, | SEQ ID NO: 2 | SEQ ID NO: 26 |
| | DnaB; 6xHis-tagged mini-intein | | SEQ ID NO: 27 |
| | | | SEQ ID NOs 61-66 |
| | | | and 79-119 |
| D.t. DnaB | Desulfofundulus | SEQ ID NO: 3 | SEQ ID NO: 28 |
| | thermosubterraneus DSM 16057, | | SEQ ID NO: 29 |
| | DnaB; intein | | |
| gp41-1 | Prochlorococcus cyanophage P- | SEQ ID NO: 4 | SEQ ID NO: 30 |
| | SSM2, gp41-1; 6xHis-tagged | | SEQ ID NO: 31 |
| | mini-intein | | |
| N.p. DnaB | Nostoc punctiforme strain ATCC | SEQ ID NO: 5 | SEQ ID NO: 32 |
| | 29133/PCC 73102, DnaB; mini- | | SEQ ID NO: 33 |
| | intein | | |
| M.t. Rec A | Mycobacterium tuberculosis | SEQ ID NO: 6 | SEQ ID NO: 34 |
| | strain ATCC 25618/H37Rv, | | SEQ ID NO: 35 |
| | RecA; mini-intein | | |
| CthBIL4 | Clostridium thermocellum, | SEQ ID NO: 7 | SEQ ID NO: 36 |
| | CthBIL4 N115D; engineered BIL | | SEQ ID NO: 37 |
| | (bacterial intein-like) | | |
| D.s. PolIII | Deinococcus swuensis, PolIII | SEQ ID NO: 8 | SEQ ID NO: 38 |
| | alpha subunit; mini-intein | | SEQ ID NO: 39 |
| C.a. DnaB | Cyanobacterium aponinum, | SEQ ID NO: 9 | SEQ ID NO: 40 |
| | DnaB; intein | | SEQ ID NO: 41 |
| DnaX (split) | Synechocystis sp. PCC6803, | N: SEQ ID NO: 10 | N: SEQ ID NO: 42 |
| | DnaX; split intein | C: SEQ ID NO: 11 | C: SEQ ID NO: 43 |
| | | (see SEQ ID NO: 1) | C: SEQ ID NO: 44 |
| | Synechocystis sp. PCC6803, | 6xHis-tagged C: | SEQ ID NO: 45 |
| | DnaX; 6xHis-tagged C-terminal | SEQ ID NO: 12 | SEQ ID NO: 46 |
| | portion | | |
| DnaB (split) | Synechocystis sp. PCC6803, | N: SEQ ID NO: 13 | N: SEQ ID NO: 47 |
| | DnaB; split intein | C: SEQ ID NO: 14 | C: SEQ ID NO: 48 |
| | | (see SEQ ID NO: 2) | C: SEQ ID NO: 49 |
| | Synechocystis sp. PCC6803, | 6xHis-tagged C: | SEQ ID NO: 50 |
| | DnaB; 6xHis-tagged C-terminal | SEQ ID NO: 15 | SEQ ID NO: 51 |
| | portion | | |
| gp41-1 | Prochlorococcus cyanophage P- | N: SEQ ID NO: 16 | N: SEQ ID NO: 52 |
| (split) | SSM2, gp41-1; split intein | C: SEQ ID NO: 17 | C: SEQ ID NO: 53 |
| | | (see SEQ ID NO: 4) | C: SEQ ID NO: 54 |
| CthBIL4 | Clostridium thermocellum, | N: SEQ ID NO: 18 | N: SEQ ID NO: 55 |
| (split C42) | CthBIL4 N115D; split engineered | C: SEQ ID NO: 19 | C: SEQ ID NO: 56 |
| | BIL, ‘C42’ version | (see SEQ ID NO: 7) | C: SEQ ID NO: 57 |
| CthBIL4 | Clostridium thermocellum, | N: SEQ ID NO: 20 | N: SEQ ID NO: 58 |
| (split C16) | CthBIL4 N115D; split engineered | C: SEQ ID NO: 21 | C: SEQ ID NO: 59 |
| | BIL, ‘C16’ version | (see SEQ ID NO: 7) | C: SEQ ID NO: 60 |
| DnaB | Synechocystis sp. PCC6803, | SEQ ID NO: 120 | SEQ ID NOs: 67-74 |
| | DnaB; 4xHisKA-tagged mini- | | |
| | intein | | |
| DnaB | Synechocystis sp. PCC6803, | SEQ ID NO: 121 | SEQ ID NO: 75 |
| | DnaB; 4xHisKH-tagged mini- | | |
| | intein | | |
| DnaB | Synechocystis sp. PCC6803, | SEQ ID NO: 122 | SEQ ID NO: 76 |
| | DnaB; 4xHisKS-tagged mini- | | |
| | intein | | |
| DnaB | Synechocystis sp. PCC6803, | SEQ ID NO: 123 | SEQ ID NO: 77 |
| | DnaB; 4xHisKT-tagged mini- | | |
| | intein | | |
| DnaB | Synechocystis sp. PCC6803, | SEQ ID NO: 124 | SEQ ID NO: 78 |
| | DnaB; 6xHis-tagged mini-intein | | |
| | variant M86 | | |

Variants of intein amino acid sequences. Intein polypeptides having variants of the amino acid sequences presented in Table 1 can be used in the methods provided herein. Preferably, such variant intein polypeptides have an activity such as the ability to ligate extein sequences and/or the ability to self-excise from extein sequences. In certain embodiments, an intein polypeptide has at least 70%, or at least 80%, or at least 90%, or at least 95% amino acid sequence identity across at least 50% (or at least 60%, or at least 70%, or at least 80%, or at least 90%) of the length of a sequence presented in Table 1, where amino acid sequence identity is determined according to Example 3. Certain intein polypeptides have at least 80% amino acid sequence identity (or at least 90% identity, or at least 95% identity) to at least 10 (or at least 20, or at least 30, or at least 40, or at least 50) contiguous amino acids of a sequence presented in Table 1, where amino acid sequence identity is determined according to Example 3. Particular intein polypeptides provided herein have at least 70% amino acid sequence identity (or at least 80% identity, or at least 90% identity, or at least 95% identity) to an amino acid sequence selected from the group consisting of amino acids 93-112 of SEQ ID NO:1, amino acids 105-124 of SEQ ID NO:2, amino acids 85-104 of SEQ ID NO:4, amino acids 1-21 of SEQ ID NO:12, amino acids 1-21 of SEQ ID NO:15, amino acids 105-124 of SEQ ID NO:120, amino acids 105-124 of SEQ ID NO:121, amino acids 105-124 of SEQ ID NO:122, amino acids 105-124 of SEQ ID NO:123, and amino acids 105-124 of SEQ ID NO:124; further intein polypeptides of the disclosure have at least 60% amino acid sequence identity (or at least 70% identity, or at least 80% identity, or at least 90% identity, or at least 95% identity) to an amino acid sequence selected from the group consisting of amino acids 88-117 of SEQ ID NO:1, amino acids 100-129 of SEQ ID NO:2, amino acids 80-109 of SEQ ID NO:4, amino acids 1-30 of SEQ ID NO:12, amino acids 1-30 of SEQ ID NO:15, amino acids 100-129 of SEQ ID NO:120, amino acids 100-129 of SEQ ID NO:121, amino acids 100-129 of SEQ ID NO:122, amino acids 100-129 of SEQ ID NO:123, and amino acids 100-129 of SEQ ID NO:124, where amino acid sequence identity is determined according to Example 3.

Tags and Other Amino Acid Sequences that can be Used with Intein Polypeptides.

Tags. Intein polypeptides to be used in the methods provided herein can be designed to include molecular moieties that aid in the purification and/or detection of such intein polypeptides and of intein fusion polypeptides comprising them. Many such moieties are known in the art; as one example, an intein polypeptide can be designed to include a polyhistidine ‘tag’ sequence—a run of six or more histidines, preferably six to ten histidine residues, and most preferably six histidines (‘6×His’)—within its amino acid sequence—such as between the N- and C-terminal portions of a mini-intein—or near the N- or C-terminus of an intein polypeptide. The presence of a polyhistidine sequence within a polypeptide allows it to be bound by cobalt- or nickel-based affinity media, and separated from other polypeptides. The polyhistidine tag sequence can be removed by exopeptidases. Another example of a tag is SpyTag, which is a peptide of 13 amino acids that is bound by the 12.3-kDa SpyCatcher protein, resulting in a covalent intermolecular isopeptide bond. A further type of tag is the AviTag™ peptide (Avidity, Aurora, Colo.), which is a target for biotinylation by biotin ligase. Additional tags include: (1) the self-cleaving N-terminal portions (N^{pro}) of polyproteins from pestiviruses such as Hog cholera virus (strain Alfort), also called classical swine fever virus (CSFV), and from border disease virus (BDV) and bovine viral diarrhea virus (BVDV), and fragments thereof; and/or (2) small ubiquitin-related modifier (SUMO) (e.g., SwissProt P55853.1). Any N-terminal tag may itself be further tagged with a polyhistidine tag such as 6×His, allowing for initial purification of the tagged polypeptide on a nickel column, followed by self-cleavage of tags such as N^{pro}, or enzymatic cleavage of the SUMO N-terminal tag by SUMO protease, respectively, and elution of the freed polypeptide from the column. In one embodiment of this method, the SUMO protease polypeptides are also fusion proteins comprising 6×His tags, allowing for a two-step purification: in the first step, the expressed 6×His-SUMO-tagged polypeptide is purified by binding to a nickel column, followed by elution from the column. In the second step, the SUMO tags on the purified polypeptides are cleaved by the 6×His-tagged SUMO protease, and the SUMO protease-polypeptide reaction mixture is run through a second nickel column, which retains the SUMO protease but allows the now untagged polypeptide to flow through.

Linkers. Intein polypeptides to be used in the methods provided herein can include linkers, which are polypeptides that are used to connect two other polypeptides. Examples of linker polypeptides that form alpha-helices are described in Amet et al., “Insertion of the designed helical linker led to increased expression of Tf-based fusion proteins,” Pharm Res 2009 March; 26(3): 523-528; doi: 10.1007/s11095-008-9767-0; Epub 2008 Nov. 11.

Additional Cleavage Sequences. Cleavage sequences are discrete amino acid sequences that can be acted upon by chemical reagents or enzymes to effect cleavage of the polypeptide containing the cleavage sequence. One or more of these sequences can be introduced between a tag (for example) and other amino acid sequences within or adjacent to an intein polypeptide, to allow the tag (or other types of amino acid sequences) to be cleaved off. An enterokinase cleavage sequence (DDDDKG, amino acids 11-16 of SEQ ID NOs 12 and 15) is included in the 6×His-tagged split intein polypeptide sequences of SEQ ID NOs 12 and 15 to allow the 6×His tag to be cleaved off. Further examples of cleavage sequences include amino sequences comprising DP, which can be cleaved by treatment with formic acid at the bond between D (Asp) and P (Pro). Additional examples are amino acid sequences cleavable by proteases such as TEV (tobacco etch virus) protease and thrombin.

Signal Peptides. Intein fusion polypeptides utilized in the methods provided herein can have or lack signal peptides. In certain embodiments, intein fusion polypeptides lack signal peptides because it is advantageous for such intein fusion polypeptides to be retained in the cytoplasm of the host cell. Signal peptides (also termed signal sequences, leader sequences, or leader peptides) are characterized structurally by a stretch of hydrophobic amino acids, approximately five to twenty amino acids long and often around ten to fifteen amino acids in length, that has a tendency to form a single alpha-helix. This hydrophobic stretch is often immediately preceded by a shorter stretch enriched in positively charged amino acids (particularly lysine). Signal peptides that are to be cleaved from the mature polypeptide typically end in a stretch of amino acids that is recognized and cleaved by signal peptidase. Signal peptides that direct insertion of polypeptides into membranes, sometimes referred to as signal anchor sequences, can lack the amino acid sequence that is cleaved by signal peptidase and in that case are retained in the polypeptide. Signal peptides can often be characterized functionally by the ability to direct transport of a polypeptide, either co-translationally or post-translationally, out of the cytoplasm and, for example, through the plasma membrane of prokaryotes (or the inner membrane of gram negative bacteria like E. coli), or into the endoplasmic reticulum of eukaryotic cells. The degree to which a signal peptide enables a polypeptide to be transported into the periplasmic space of a host cell like E. coli, for example, can be determined by separating periplasmic proteins from proteins retained in the cytoplasm (see Example 12 of WO2014025663A1).

### II. Polypeptides and Proteins to be Produced by the Methods

The methods provided herein comprise the use of intein amino acid sequences included within an intein fusion polypeptide sequence to be cleaved, and the self-excision of the intein amino sequences from the remainder of the intein fusion polypeptide to generate a target polypeptide or protein. The target polypeptides and proteins that can be produced using the methods of the disclosure can comprise any, or more than one, of the following: alpha-1-antitrypsin; 2C4 (a monoclonal antibody against HER2); activin; addressins; alkaline phosphatase; anti-CD11a; anti-CD18; anti-CD20; anti-clotting factors such as Protein C; anti-HER-2 antibody; anti-IgE; anti-IgG; anti-VEGF; antibodies and antibody fragments; antibodies to ErbB2 domain(s) such as 2C4 (WO 01/00245 hybridoma ATCC HB-12697), which binds to a region in the extracellular domain of ErbB2 (e.g., any one or more residues in the region from about residue 22 to about residue 584 of ErbB2, inclusive); Apo2 ligand (Apo2L); atrial natriuretic factor; BDNF; beta-lactamase; bombesin; bone morphogenetic protein (BMP); botulinum toxin; brain IGF-I; calcitonin; cardiotrophins (cardiac hypertrophy factor) such as cardiotrophin-1 (CT-1); CD proteins such as CD3, CD4, CD8, and CD19; clotting factors such as factor VIIIC, factor IX, tissue factor, and von Willebrands factor; colony stimulating factors (CSFs), e.g., M-CSF, GM-CSF, and G-CSF; cytokines; decay-accelerating factor; des(1-3)-IGF-I (brain IGF-I); DNase; enkephalinase; epidermal growth factor (EGF); erythropoietin; fibroblast growth factors, such as aFGF and bFGF; follicle-stimulating hormone; glucagon; gp120; ghrelin; growth hormone, including human growth hormone or bovine growth hormone; growth-hormone releasing factor; hemopoietic growth factor; homing receptors; HSA; IGF-I; IGF-II; immunotoxins; inhibin; insulin chains (insulin A-chain, insulin B-chain) or proinsulin; insulin-like growth factor binding proteins; insulin-like growth factor-I and -II (IGF-I and IGF-II); integrin; interferons, such as interferon-alpha, -beta, and -gamma; interleukins (ILs), e.g., IL-1 to IL-10; leptin; lipoproteins; lung surfactant; luteinizing hormone; metreleptin; mouse gonadotropin-associated peptide; mullerian-inhibiting substance; nerve growth factor (NGF); neurotrophic factors, such as brain-derived neurotrophic factor (BDNF), neurotrophin-3, -4, -5, or -6 (NT-3, NT-4, NT-5, or NT-6); osteoinductive factors; parathyroid hormone; plasminogen activator, such as urokinase or human urine or tissue-type plasminogen activator (t-PA); platelet-derived growth factor (PDGF); prorelaxin; protein A or D; receptors for hormones or growth factors; regulatory proteins; relaxin A-chain; relaxin B-chain; rennin; rheumatoid factors; serum albumin, such as human serum albumin (HSA) or bovine serum albumin (BSA); superoxide dismutase; surface-membrane proteins; T-cell receptors; TGF-beta; thrombin; thrombopoietin; thyroid-stimulating hormone; transforming growth factor (TGF) such as TGF-alpha and TGF-beta, including TGF-1, TGF-2, TGF-3, TGF-4, or TGF-5; transport proteins; tumor necrosis factor-alpha and -beta; urokinase; vascular endothelial growth factor (VEGF); viral antigens such as, for example, a portion of the AIDS envelope; fragments of any of the above; and any of the above or a fragment thereof covalently bound to one or more of the proteins above or fragments thereof or functional domains such as: an antibody Fc domain, an antibody single-chain variable fragment (scFv), a domain with enzymatic activity (such as a glycoside hydrolase domain or a kinase domain), an EVH1 (Ena/Vasp homology, or WH1) domain, a PAS (Per-Arnt-Sim) domain, a PDZ domain, a POU (Pit-1, Oct, Unc-86) domain, an SPR (Spread, Sprouty) domain, a VWFC (Von Willebrand factor, type C or VWC) domain, or a zinc-finger domain (for example, a RING-finger domain).

Disulfide Bonds. The target polypeptides and proteins produced by the methods provided herein are in some instances polypeptides and proteins that can form disulfide bonds. Disulfide bonds are covalent bonds between sulfur atoms, represented as R—S—S—R′, formed for example between the thiol groups of cysteine residues present in polypeptides. The number of disulfide bonds for a polypeptide or protein is the total number of intramolecular and intermolecular disulfide bonds formed by that polypeptide or protein when it is present in a functional product. For example, when human IgG antibody light chains and heavy chains are coexpressed, an antibody light chain typically can form three disulfide bonds (two intramolecular bonds and one intermolecular bond), and an antibody heavy chain typically can form seven disulfide bonds (four intramolecular bonds and three intermolecular bonds). In certain embodiments, a polypeptide or protein produced by methods herein can form at least one and fewer than twenty disulfide bonds, or at least two and fewer than seventeen disulfide bonds, or at least seventeen and fewer than fifty disulfide bonds, or at least three and fewer than ten disulfide bonds, or at least three and fewer than eight disulfide bonds, or is a polypeptide or protein that can form a number of disulfide bonds selected from the group consisting of one, two, three, four, five, six, seven, eight, and nine disulfide bonds.

Chaperones. In some embodiments, the intein fusion polypeptides are coexpressed with other gene products, such as chaperones, that are beneficial to the production of the target polypeptide or protein. Chaperones are proteins that assist the folding or unfolding, and/or the assembly or disassembly, of other gene products, but do not occur in the resulting monomeric or multimeric gene product structures when the structures are performing their normal biological functions (having completed the processes of folding and/or assembly). Chaperones can be expressed from an inducible promoter or a constitutive promoter within an expression construct, or can be expressed from the host cell chromosome; preferably, expression of chaperone protein(s) in the host cell is at a sufficiently high level to produce coexpressed target polypeptides that are properly folded and/or assembled into the target protein. Examples of chaperones present in E. coli host cells are the folding factors DnaK/DnaJ/GrpE, DsbC/DsbG, GroEL/GroES, IbpA/IbpB, Skp, Tig (trigger factor), and FkpA, which have been used to prevent protein aggregation of cytoplasmic or periplasmic proteins. DnaK/DnaJ/GrpE, GroEL/GroES, and ClpB can function synergistically in assisting protein folding and therefore expression of these chaperones in combinations has been shown to be beneficial for protein expression (Makino et al., “Strain engineering for improved expression of recombinant proteins in bacteria,” Microb Cell Fact 2011 May 14; 10: 32). When expressing eukaryotic proteins in prokaryotic host cells, a eukaryotic chaperone protein, such as protein disulfide isomerase (PDI) from the same or a related eukaryotic species, or from Humicola insolens, is in certain embodiments coexpressed or inducibly coexpressed with the desired gene product.

### III. Expression Constructs

Expression constructs are polynucleotides designed for the expression of one or more gene products of interest, such as intein fusion polypeptides comprising intein amino acid sequences and the amino acid sequence(s) of the target polypeptide(s) or protein(s). Certain gene products of interest are ‘heterologous’ gene products, that are derived from species that are different from that of the host cell in which they are expressed, and/or are heterologous gene products that are not natively expressed from the promoter(s) utilized within the expression construct, and/or are modified gene products that have been designed to include differences from naturally occurring forms of such gene products. Expression constructs comprising polynucleotides encoding heterologous and/or modified gene products, or comprising a combination of polynucleotides that were derived from organisms of different species, or comprising polynucleotides that have been modified to differ from naturally occurring polynucleotides, are not naturally occurring molecules. Expression constructs can be integrated into a host cell chromosome, or maintained within the host cell as polynucleotide molecules replicating independently of the host cell chromosome, such as plasmids or artificial chromosomes. An example of an expression construct is a polynucleotide resulting from the insertion of one or more polynucleotide sequences into a host cell chromosome, where the inserted polynucleotide sequences alter the expression of chromosomal coding sequences. An expression vector is a plasmid expression construct specifically used for the expression of one or more gene products. One or more expression constructs can be integrated into a host cell chromosome or be maintained on an extrachromosomal polynucleotide such as a plasmid or artificial chromosome. In certain embodiments, the expression construct is a dual-promoter expression vector such as those described in US2015353940A1 and WO2016205570A1.

Expression constructs can comprise certain polynucleotide elements, such as origins of replication, selectable markers, promoters such as constitutive or inducible promoters (described further below), ribosome binding sites, and multiple cloning sites. Examples of these polynucleotide elements are well known in the art, and further descriptions of them can be found in the following patent publications and application(s), all of which are expressly incorporated by reference herein: U.S. Pat. No. 9,617,335B2 and WO 2014/025663A1, “Inducible Coexpression System”; US 2015/353940A1 and WO 2016/205570A1, “Vectors for Use in an Inducible Coexpression System”; and International Application PCT/US2016/067064, “Cytoplasmic Expression System”.

Inducible promoter. As described further below, there are several different inducible promoters that can be included in expression constructs as part of the expression systems described herein. Preferred inducible promoters include those described in Table 2.

TABLE 2
E. coli Sugar-Responsive Transcription Factors
and the Sugar-Inducible Promoters Regulated by Them

| | Transcription | |
|---|---|---|
| | Factor(s) | |
| Sugar(s) | (Synonym) | Genes with Sugar-Inducible Promoter(s) |
| D-Allose | AlsR (RpiR) | alsRBACE; rpiB |
| L-Arabinose | AraC | araBAD; araC; araE; araFGH; araJ |
| N,N′-Diacetyl- | ChbR (CelD) | chbBCARFG |
| Chitobiose | | |
| Fructose | Cra (FruR) | eno; epd-pgk-fbaA; fbaB; fruBKA; gapA-yeaD; glk; |
| [see note] | | gpmM-envC-yibQ; hypF; mpl; mtlADR; pdeL; pfkA; |
| | | ppc; ptsHI-crr (p1); pykF; tpiA |
| L-Fucose | FucR | fucAO; fucPIKUR |
| D-Galactose | GalR and GalS | galETKM; galP; galR; galS; mglBAC |
| D-Glucose | Cra and SgrR | sgrST-setA |
| D-Glucose | CreB | ackA-pta; cbrA; cbrB; creD; nudF-yqiB-cpdA-yqiA- |
| | | parE; recG; talA |
| D-Glucose | SgrR | alaC; sgrR-sroA-thiBPQ |
| Glucose-6-P | UhpA | uhpT |
| Lactose, | LacI | lacZYA |
| Allolactose | | |
| Lactose, | EbgR | ebgAC |
| Lactulose | | |
| Maltose | MalI | malI; malXY |
| Maltose, | MalT | malEFG; malK-lamB-malM; malPQ; malS; malZ |
| Maltotriose | | |
| Melibiose | MelR | melAB; melR |
| L-Rhamnose | RhaR and RhaS | rhaSR |
| L-Rhamnose | RhaS | rhaBAD; rhaT |
| D-Ribose | RbsR | rbsDACBKR |
| Sulfoquinovose | CsqR (YihW) | yihUTS; yihV; yihW |
| D-Trehalose | TreR | treBC |
| D-Xylose | XylR | xylAB; xylE; xylFGHR |

[Note]
Cra acts as an activator (no inducer required) or as a repressor (inducer required for derepression) of many different promoters. For some Cra-regulated promoters, binding or derepression of Cra plus the binding of (or derepression of) other transcription factors is required for expression. The promoters listed here are those where the addition of fructose, in the absence of repressive sugars such as glucose, should be sufficient to result in Cra derepression and expression of the promoter.

Certain preferred inducible promoters share at least 80% polynucleotide sequence identity (more preferably, at least 90% identity, and most preferably, at least 95% identity) to at least 30 (more preferably, at least 40, and most preferably, at least 50) contiguous bases of a promoter polynucleotide sequence as defined in Table 1 of WO 2014/025663A1, where percent polynucleotide sequence identity is determined using the methods of Example 3. Preferred inducible promoters have at least 75% (more preferably, at least 100%, and most preferably, at least 110%) of the strength of the corresponding ‘wild-type’ inducible promoter of E. coli K-12 substrain MG1655, as determined using the quantitative PCR method of De Mey et al. “Promoter knock-in: a novel rational method for the fine tuning of genes,” BMC Biotechnol 2010 Mar. 24; 10: 26 (see Example 8A of WO 2014/025663A1). Within the expression construct, an inducible promoter is placed 5′ to (or ‘upstream of’) the coding sequence for the gene product that is to be inducibly expressed, so that the presence of the inducible promoter will direct transcription of the gene product coding sequence in a 5′ to 3′ direction relative to the coding strand of the polynucleotide encoding the gene product. The gene products expressed from the inducible promoters within expression constructs are not the gene products natively expressed from these inducible promoters; rather, they are heterologous gene products, with the result that the expression constructs comprising heterologous gene products expressed from inducible promoters are necessarily artificial constructs not found in nature.

Inducible Promoters. The following is a description of inducible promoters that can be used in expression constructs for expression of gene products, along with some of the genetic modifications that can be made to host cells that contain such expression constructs. Examples of these inducible promoters and related genes are, unless otherwise specified, those derived from Escherichia coli (E. coli) strain MG1655 (American Type Culture Collection deposit ATCC 700926), which is a substrain of E. coli K-12 (American Type Culture Collection deposit ATCC 10798). Table 1 of International Application PCT/US13/53562 (WO 2014/025663A1) lists the genomic locations, in E. coli MG1655, of the nucleotide sequences for these examples of inducible promoters and related genes; the WO2014025663A1 publication is incorporated by reference in its entirety herein. Nucleotide and other genetic sequences, referenced by genomic location as in Table 1 of WO 2014/025663A1, are expressly incorporated by reference herein. Additional information about E. coli promoters, genes, and strains described herein can be found in many public sources, including the online EcoliWiki resource, located at ecoliwiki.net.

Arabinose promoter. (As used herein, ‘arabinose’ means L-arabinose.) Several E. coli operons involved in arabinose utilization are inducible by arabinose—araBAD, araC, araE, and araFGH—but the terms ‘arabinose promoter’ and ‘ara promoter’ are typically used to designate the araBAD promoter. Several additional terms have been used to indicate the E. coli araBAD promoter, such as Pₐᵣₐ, P_{araB}, P_{araBAD}, and P_{BAD}. The use herein of ‘ara promoter’ or any of the alternative terms given above, means the E. coli araBAD promoter. As can be seen from the use of another term, ‘araC-araBAD promoter’, the araBAD promoter is considered to be part of a bidirectional promoter, with the araBAD promoter controlling expression of the araBAD operon in one direction, and the araC promoter, in close proximity to and on the opposite strand from the araBAD promoter, controlling expression of the araC coding sequence in the other direction. The AraC protein is both a positive and a negative transcriptional regulator of the araBAD promoter. In the absence of arabinose, the AraC protein represses transcription from P_{BAD}, but in the presence of arabinose, the AraC protein, which alters its conformation upon binding arabinose, becomes a positive regulatory element that allows transcription from P_{BAD}. The araBAD operon encodes proteins that metabolize L-arabinose by converting it, through the intermediates L-ribulose and L-ribulose-phosphate, to D-xylulose-5-phosphate. For the purpose of maximizing induction of expression from an arabinose-inducible promoter, it is useful to eliminate or reduce the function of AraA, which catalyzes the conversion of L-arabinose to L-ribulose, and optionally to eliminate or reduce the function of at least one of AraB and AraD, as well. Eliminating or reducing the ability of host cells to decrease the effective concentration of arabinose in the cell, by eliminating or reducing the cell's ability to convert arabinose to other sugars, allows more arabinose to be available for induction of the arabinose-inducible promoter. The genes encoding the transporters which move arabinose into the host cell are araE, which encodes the low-affinity L-arabinose proton symporter, and the araFGH operon, which encodes the subunits of an ABC superfamily high-affinity L-arabinose transporter. Other proteins which can transport L-arabinose into the cell are certain mutants of the LacY lactose permease: the LacY(A177C) and the LacY(A177V) proteins, having a cysteine or a valine amino acid instead of alanine at position 177, respectively (Morgan-Kiss et al., “Long-term and homogeneous regulation of the Escherichia coli araBAD promoter by use of a lactose transporter of relaxed specificity,” Proc Natl Acad Sci USA 2002 May 28; 99(11): 7373-7377). In order to achieve homogenous induction of an arabinose-inducible promoter, it is useful to make transport of arabinose into the cell independent of regulation by arabinose. This can be accomplished by eliminating or reducing the activity of the AraFGH transporter proteins and altering the expression of araE so that it is only transcribed from a constitutive promoter. Constitutive expression of araE can be accomplished by eliminating or reducing the function of the native araE gene, and introducing into the cell an expression construct which includes a coding sequence for the AraE protein expressed from a constitutive promoter. Alternatively, in a cell lacking AraFGH function, the promoter controlling expression of the host cell's chromosomal araE gene can be changed from an arabinose-inducible promoter to a constitutive promoter. In similar manner, as additional alternatives for homogenous induction of an arabinose-inducible promoter, a host cell that lacks AraE function can have any functional AraFGH coding sequence present in the cell expressed from a constitutive promoter. As another alternative, it is possible to express both the araE gene and the araFGH operon from constitutive promoters, by replacing the native araE and araFGH promoters with constitutive promoters in the host chromosome. It is also possible to eliminate or reduce the activity of both the AraE and the AraFGH arabinose transporters, and in that situation to use a mutation in the LacY lactose permease that allows this protein to transport arabinose. Since expression of the lacY gene is not normally regulated by arabinose, use of a LacY mutant such as LacY(A177C) or LacY(A177V), will not lead to the ‘all or none’ induction phenomenon when the arabinose-inducible promoter is induced by the presence of arabinose. Because the LacY(A177C) protein appears to be more effective in transporting arabinose into the cell, use of polynucleotides encoding the LacY(A177C) protein is preferred to the use of polynucleotides encoding the LacY(A177V) protein.

Propionate promoter. The ‘propionate promoter’ or ‘prp promoter’ is the promoter for the E. coli prpBCDE operon, and is also called P_{prpB}. Like the ara promoter, the prp promoter is part of a bidirectional promoter, controlling expression of the prpBCDE operon in one direction, and with the prpR promoter controlling expression of the prpR coding sequence in the other direction. The PrpR protein is the transcriptional regulator of the prp promoter, and activates transcription from the prp promoter when the PrpR protein binds 2-methylcitrate (‘2-MC’). Propionate (also called propanoate) is the ion, CH₃CH₂COO⁻, of propionic acid (or ‘propanoic acid’), and is the smallest of the ‘fatty’ acids having the general formula H(CH₂)—COOH that shares certain properties of this class of molecules: producing an oily layer when salted out of water and having a soapy potassium salt. Commercially available propionate is generally sold as a monovalent cation salt of propionic acid, such as sodium propionate (CH₃CH₂COONa), or as a divalent cation salt, such as calcium propionate (Ca(CH₃CH₂COO)₂). Propionate is membrane-permeable and is metabolized to 2-MC by conversion of propionate to propionyl-CoA by PrpE (propionyl-CoA synthetase), and then conversion of propionyl-CoA to 2-MC by PrpC (2-methylcitrate synthase). The other proteins encoded by the prpBCDE operon, PrpD (2-methylcitrate dehydratase) and PrpB (2-methylisocitrate lyase), are involved in further catabolism of 2-MC into smaller products such as pyruvate and succinate. In order to maximize induction of a propionate-inducible promoter by propionate added to the cell growth medium, it is therefore desirable to have a host cell with PrpC and PrpE activity, to convert propionate into 2-MC, but also having eliminated or reduced PrpD activity, and optionally eliminated or reduced PrpB activity as well, to prevent 2-MC from being metabolized. Another operon encoding proteins involved in 2-MC biosynthesis is the scpA-argK-scpBC operon, also called the sbm-ygfDGH operon. These genes encode proteins required for the conversion of succinate to propionyl-CoA, which can then be converted to 2-MC by PrpC. Elimination or reduction of the function of these proteins would remove a parallel pathway for the production of the 2-MC inducer, and thus might reduce background levels of expression of a propionate-inducible promoter, and increase sensitivity of the propionate-inducible promoter to exogenously supplied propionate. It has been found that a deletion of sbm-ygfD-ygfG-ygfH-ygfI, introduced into E. coli BL21(DE3) to create strain JSB (Lee and Keasling, “A propionate-inducible expression system for enteric bacteria,” Appl Environ Microbiol 2005 November; 71(11): 6856-6862), was helpful in reducing background expression in the absence of exogenously supplied inducer, but this deletion also reduced overall expression from the prp promoter in strain JSB. It should be noted, however, that the deletion sbm-ygfD-ygfG-ygfH-ygfI also apparently affects ygfI, which encodes a putative LysR-family transcriptional regulator of unknown function. The genes sbm-ygfDGH are transcribed as one operon, and ygfI is transcribed from the opposite strand. The 3′ ends of the ygfH and ygfI coding sequences overlap by a few base pairs, so a deletion that takes out all of the sbm-ygfDGH operon apparently takes out ygfI coding function as well. Eliminating or reducing the function of a subset of the sbm-ygfDGH gene products, such as YgfG (also called ScpB, methylmalonyl-CoA decarboxylase), or deleting the majority of the sbm-ygfDGH (or scpA-argK-scpBC) operon while leaving enough of the 3′ end of the ygfH (or scpC) gene so that the expression of ygfI is not affected, could be sufficient to reduce background expression from a propionate-inducible promoter without reducing the maximal level of induced expression.

Rhamnose promoter. (As used herein, ‘rhamnose’ means L-rhamnose.) The ‘rhamnose promoter’ or ‘rha promoter’, or P_{rhaSR}, is the promoter for the E. coli rhaSR operon. Like the ara and prp promoters, the rha promoter is part of a bidirectional promoter, controlling expression of the rhaSR operon in one direction, and with the rhaBAD promoter controlling expression of the rhaBAD operon in the other direction. The rha promoter, however, has two transcriptional regulators involved in modulating expression: RhaR and RhaS. The RhaR protein activates expression of the rhaSR operon in the presence of rhamnose, while RhaS protein activates expression of the L-rhamnose catabolic and transport operons, rhaBAD and rhaT, respectively (Wickstrum et al., “The AraC/XylS family activator RhaS negatively autoregulates rhaSR expression by preventing cyclic AMP receptor protein activation,” J Bacteriol 2010 January; 192(1): 225-232). Although the RhaS protein can also activate expression of the rhaSR operon, in effect RhaS negatively autoregulates this expression by interfering with the ability of the cyclic AMP receptor protein (CRP) to coactivate expression with RhaR to a much greater level. The rhaBAD operon encodes the rhamnose catabolic proteins RhaA (L-rhamnose isomerase), which converts L-rhamnose to L-rhamnulose; RhaB (rhamnulokinase), which phosphorylates L-rhamnulose to form L-rhamnulose-1-P; and RhaD (rhamnulose-1-phosphate aldolase), which converts L-rhamnulose-1-P to L-lactaldehyde and DHAP (dihydroxyacetone phosphate). To maximize the amount of rhamnose in the cell available for induction of expression from a rhamnose-inducible promoter, it is desirable to reduce the amount of rhamnose that is broken down by catalysis, by eliminating or reducing the function of RhaA, or optionally of RhaA and at least one of RhaB and RhaD. E. coli cells can also synthesize L-rhamnose from alpha-D-glucose-1-P through the activities of the proteins Rm1A, Rm1B, Rm1C, and Rm1D (also called RfbA, RfbB, RfbC, and RfbD, respectively) encoded by the rmlBDACX (or rfbBDACX) operon. To reduce background expression from a rhamnose-inducible promoter, and to enhance the sensitivity of induction of the rhamnose-inducible promoter by exogenously supplied rhamnose, it could be useful to eliminate or reduce the function of one or more of the Rm1A, Rm1B, Rm1C, and Rm1D proteins. L-rhamnose is transported into the cell by RhaT, the rhamnose permease or L-rhamnose:proton symporter. As noted above, the expression of RhaT is activated by the transcriptional regulator RhaS. To make expression of RhaT independent of induction by rhamnose (which induces expression of RhaS), the host cell can be altered so that all functional RhaT coding sequences in the cell are expressed from constitutive promoters. Additionally, the coding sequences for RhaS can be deleted or inactivated, so that no functional RhaS is produced. By eliminating or reducing the function of RhaS in the cell, the level of expression from the rhaSR promoter is increased due to the absence of negative autoregulation by RhaS, and the level of expression of the rhamnose catalytic operon rhaBAD is decreased, further increasing the ability of rhamnose to induce expression from the rha promoter.

Xylose promoter. (As used herein, ‘xylose’ means D-xylose.) The xylose promoter, or ‘xyl promoter’, or P_{xylA}, means the promoter for the E. coli xylAB operon. The xylose promoter region is similar in organization to other inducible promoters in that the xylAB operon and the xylFGHR operon are both expressed from adjacent xylose-inducible promoters in opposite directions on the E. coli chromosome (Song and Park, “Organization and regulation of the D-xylose operons in Escherichia coli K-12: XylR acts as a transcriptional activator,” J Bacteriol. 1997 November; 179(22): 7025-7032). The transcriptional regulator of both the P_{xylA} and P_{xylF} promoters is XylR, which activates expression of these promoters in the presence of xylose. The xylR gene is expressed either as part of the xylFGHR operon or from its own weak promoter, which is not inducible by xylose, located between the xylH and xylR protein-coding sequences. D-xylose is catabolized by XylA (D-xylose isomerase), which converts D-xylose to D-xylulose, which is then phosphorylated by XylB (xylulokinase) to form D-xylulose-5-P. To maximize the amount of xylose in the cell available for induction of expression from a xylose-inducible promoter, it is desirable to reduce the amount of xylose that is broken down by catalysis, by eliminating or reducing the function of at least XylA, or optionally of both XylA and XylB. The xylFGHR operon encodes XylF, XylG, and XylH, the subunits of an ABC superfamily high-affinity D-xylose transporter. The xylE gene, which encodes the E. coli low-affinity xylose-proton symporter, represents a separate operon, the expression of which is also inducible by xylose. To make expression of a xylose transporter independent of induction by xylose, the host cell can be altered so that all functional xylose transporters are expressed from constitutive promoters. For example, the xylFGHR operon could be altered so that the xylFGH coding sequences are deleted, leaving XylR as the only active protein expressed from the xylose-inducible P_{xylF} promoter, and with the xylE coding sequence expressed from a constitutive promoter rather than its native promoter. As another example, the xylR coding sequence is expressed from the P_{xylA} or the P_{xylF} promoter in an expression construct, while either the xylFGHR operon is deleted and xylE is constitutively expressed, or alternatively an xylFGH operon (lacking the xylR coding sequence since that is present in an expression construct) is expressed from a constitutive promoter and the xylE coding sequence is deleted or altered so that it does not produce an active protein.

Lactose promoter. The term ‘lactose promoter’ refers to the lactose-inducible promoter for the lacZYA operon, a promoter which is also called lacZp1; this lactose promoter is located at ca. 365603-365568 (minus strand, with the RNA polymerase binding (‘−35’) site at ca. 365603-365598, the Pribnow box (‘-10’) at 365579-365573, and a transcription initiation site at 365567) in the genomic sequence of the E. coli K-12 substrain MG1655 (NCBI Reference Sequence NC_000913.2, 11 Jan. 2012). In some embodiments, expression systems can comprise a lactose-inducible promoter such as the lacZYA promoter. In other embodiments, the expression systems comprise one or more inducible promoters that are not lactose-inducible promoters.

Alkaline phosphatase promoter. The terms ‘alkaline phosphatase promoter’ and ‘phoA promoter’ refer to the promoter for the phoApsiF operon, a promoter which is induced under conditions of phosphate starvation. The phoA promoter region is located at ca. 401647-401746 (plus strand, with the Pribnow box (‘−10’) at 401695-401701 (Kikuchi et al., “The nucleotide sequence of the promoter and the amino-terminal region of alkaline phosphatase structural gene (phoA) of Escherichia coli,” Nucleic Acids Res 1981 Nov. 11; 9(21): 5671-5678) in the genomic sequence of the E. coli K-12 substrain MG1655 (NCBI Reference Sequence NC_000913.3, 16 Dec. 2014). The transcriptional activator for the phoA promoter is PhoB, a transcriptional regulator that, along with the sensor protein PhoR, forms a two-component signal transduction system in E. coli. PhoB and PhoR are transcribed from the phoBR operon, located at ca. 417050-419300 (plus strand, with the PhoB coding sequence at 417,142-417,831 and the PhoR coding sequence at 417,889-419,184) in the genomic sequence of the E. coli K-12 substrain MG1655 (NCBI Reference Sequence NC_000913.3, 16 Dec. 2014). The phoA promoter differs from the inducible promoters described above in that it is induced by the lack of a substance—intracellular phosphate—rather than by the addition of an inducer. For this reason the phoA promoter is generally used to direct transcription of gene products that are to be produced at a stage when the host cells are depleted for phosphate, such as the later stages of fermentation. In some embodiments, expression systems can comprise a phoA promoter. In other embodiments, the expression systems comprise one or more inducible promoters that are not phoA promoters.

### IV. Host Cells

For production of intein fusion polypeptides to be used as described herein, host cells can be any cell capable of expressing such polypeptides, such as single-celled organisms, isolated cells grown in culture, or isolated cells derived from a multicellular organism. Examples of host cells are provided that allow for efficient inducible expression of such polypeptides.

Particularly suitable host cells are capable of growth at high cell density in fermentation culture, and can produce intein fusion polypeptides in oxidizing host cell cytoplasm through highly controlled inducible gene expression. Host cells with these qualities are produced by combining some or all of the following characteristics. (1) The host cells are genetically modified to have an oxidizing cytoplasm, through increasing the expression or function of oxidizing polypeptides in the cytoplasm, and/or by decreasing the expression or function of reducing polypeptides in the cytoplasm. Increased expression of the cysteine oxidase DsbA, the disulfide isomerase DsbC, or combinations of the Dsb proteins, which are all normally transported into the periplasm, has been utilized in the expression of heterologous proteins that require disulfide bonds (Makino et al., “Strain engineering for improved expression of recombinant proteins in bacteria,” Microb Cell Fact 2011 May 14; 10: 32). It is also possible to express cytoplasmic forms of these Dsb proteins, such as a cytoplasmic version of DsbC (‘cDsbC’), for example having an N-terminal truncation of twenty amino acids, which lacks a signal peptide and therefore is not transported into the periplasm. Cytoplasmic Dsb proteins such as cDsbC are useful for making the cytoplasm of the host cell more oxidizing and thus more conducive to the formation of disulfide bonds in heterologous proteins produced in the cytoplasm. The host cell cytoplasm can also be made more oxidizing by altering the thioredoxin and the glutaredoxin/glutathione enzyme systems directly: mutant strains defective in glutathione reductase (gor) or glutathione synthetase (gshB), together with thioredoxin reductase (trxB), render the cytoplasm oxidizing. These strains are unable to reduce ribonucleotides and therefore cannot grow in the absence of exogenous reductant, such as dithiothreitol (DTT). Suppressor mutations (ahpC* or ahpC^{Δ}) in the gene ahpC, which encodes the peroxiredoxin AhpC, convert it to a disulfide reductase that generates reduced glutathione, allowing the channeling of electrons onto the enzyme ribonucleotide reductase and enabling the cells defective in gor and trxB, or defective in gshB and trxB, to grow in the absence of DTT. A different class of mutated forms of AhpC can allow strains, defective in the activity of gamma-glutamylcysteine synthetase (gshA) and defective in trxB, to grow in the absence of DTT; these include AhpC V164G, AhpC S71F, AhpC E173/S71F, AhpC E171Ter, and AhpC dup162-169 (Faulkner et al., “Functional plasticity of a peroxidase allows evolution of diverse disulfide-reducing pathways,” Proc Natl Acad Sci USA 2008 May 6; 105(18): 6735-6740, Epub 2008 May 2). (2) Optionally, host cells can also be genetically modified to express chaperones and/or cofactors that assist in the production of the desired gene product(s), and/or to glycosylate polypeptide gene products. (3) The host cells may also contain additional genetic modifications designed to improve certain aspects of gene product expression from the expression construct(s). In particular embodiments, the host cells (A) have an alteration of gene function of at least one gene encoding a transporter protein for an inducer of at least one inducible promoter, and as another example, wherein the gene encoding the transporter protein is selected from the group consisting of araE, araF, araG, araH, rhaT, xylF, xylG, and xylH, or particularly is araE, or wherein the alteration of gene function more particularly is expression of araE from a constitutive promoter; and/or (B) have a reduced level of gene function of at least one gene encoding a protein that metabolizes an inducer of at least one inducible promoter, and as further examples, wherein the gene encoding a protein that metabolizes an inducer of at least one inducible promoter is selected from the group consisting of araA, araB, araD, prpB, prpD, rhaA, rhaB, rhaD, xylA, and xylB; and/or (C) have a reduced level of gene function of at least one gene encoding a protein involved in biosynthesis of an inducer of at least one inducible promoter, which gene in further embodiments is selected from the group consisting of scpA/sbm, argK/ygfD, scpB/ygfG, scpC/ygfH, rmlA, rmlB, rmlC, and rmlD.

In certain embodiments, the host cells are microbial cells such as yeasts (Saccharomyces, Schizosaccharomyces, etc.) or bacterial cells, or are gram-positive bacteria or gram-negative bacteria, or are E. coli, or are an E. coli B strain, or are E. coli B strain 521 cells, or are E. coli B strain 522 cells. E. coli 521 and 522 cells have the following genotypes:
E. coli 521: ΔaraBAD fhuA2 [lon] ompT ahpC^{Δ} gal λatt::pNEB3-r1-cDsbC (Spec, lacI) ΔtrxB sulA11 R(mcr-73::miniTn10--Tet^{S})2 [dcm] R(zgb-210::Tn10--Tet^{S}) ΔaraEp::J23104 ΔscpA-argK-scpBC endA1 rpsL-Arg43 Δgor Δ(mcrC-mrr)114::IS10
E. coli 522: ΔaraBAD fhuA2 prpD [ion] ompT ahpC^{Δ} gal λatt::pNEB3-r1-cDsbC (Spec, lacI) ΔtrxB sulA11 R(mcr-73::miniTn10--Tet^{S})2 [dcm] R(zgb-210::Tn10--Tet^{S}) ΔaraEp::J23104 ΔscpA-argK-scpBC endA1 rpsL-Arg43 Δgor Δ(mcrC-mrr)114::IS10

In growth experiments with E. coli host cells having oxidizing cytoplasm, we have determined that E. coli B strains with oxidizing cytoplasm are able to grow to much higher cell densities than a corresponding E. coli K strain. Other suitable strains include E. coli B strains SHuffle® Express (NEB Catalog No. C3028H) and SHuffle® T7 Express (NEB Catalog No. C3029H), and the E. coli K strain SHuffle® T7 (NEB Catalog No. C3026H).

Prokaryotic host cells. In some embodiments, the host cells are prokaryotic host cells. Prokaryotic host cells can include archaea (such as Haloferax volcanii, Sulfolobus solfataricus), Gram-positive bacteria (such as Bacillus subtilis, Bacillus licheniformis, Brevibacillus choshinensis, Lactobacillus brevis, Lactobacillus buchneri, Lactococcus lactis, and Streptomyces lividans), or Gram-negative bacteria, including Alphaproteobacteria (Agrobacterium tumefaciens, Caulobacter crescentus, Rhodobacter sphaeroides, and Sinorhizobium meliloti), Betaproteobacteria (Alcaligenes eutrophus), and Gammaproteobacteria (Acinetobacter calcoaceticus, Azotobacter vinelandii, Escherichia coli, Pseudomonas aeruginosa, and Pseudomonas putida). Preferred host cells include Gammaproteobacteria of the family Enterobacteriaceae, such as Enterobacter, Erwinia, Escherichia (including E. coli), Klebsiella, Proteus, Salmonella (including Salmonella typhimurium), Serratia (including Serratia marcescans), and Shigella.

Eukaryotic host cells. Many additional types of host cells can be used in the disclosed methods, including eukaryotic cells such as yeast (Candida shehatae, Kluyveromyces lactis, Kluyveromyces fragilis, other Kluyveromyces species, Pichia pastoris, Saccharomyces cerevisiae, Saccharomyces pastorianus also known as Saccharomyces carlsbergensis, Schizosaccharomyces pombe, Dekkera/Brettanomyces species, and Yarrowia lipolytica); other fungi (Aspergillus nidulans, Aspergillus niger, Neurospora crassa, Penicillium, Tolypocladium, Trichoderma reesia); insect cell lines (Drosophila melanogaster Schneider 2 cells and Spodoptera frugiperda Sf9 cells); and mammalian cell lines including immortalized cell lines (Chinese hamster ovary (CHO) cells, HeLa cells, baby hamster kidney (BHK) cells, monkey kidney cells (COS), human embryonic kidney (HEK, 293, or HEK-293) cells, and human hepatocellular carcinoma cells (Hep G2)). The above host cells are available from the American Type Culture Collection.

### V. Methods for Growing Host Cells.

Small-Volume Growth. Host cells used to produce intein fusion polypeptides can be grown in small volumes for the purpose of testing growth or induction conditions, or for the production of multiple different gene products, etc. The nature of the experiments to be performed will determine the volume that the host cells are to be grown in, such as one mL up to one liter, or between 5 mL and 500 mL, or any convenient volume. In certain embodiments, the vessel in which the host cells are grown is moved repeatedly in order to agitate the growth medium and thus provide oxygen to the host cells. Host cells are grown in a medium containing suitable nutrients and any antibiotics required to select for the retention by the host cells of expression constructs that provide antibiotic resistance. To determine the appropriate amount of inducer to be used to induce expression of inducible expression constructs present in cells, experiments can advantageously be performed with host cells grown in small volumes such as in multiwell plates.

Fermentation. The fermentation processes involved in the production of recombinant proteins will use a mode of operation which falls within one of the following categories: (1) discontinuous (batch process) operation, (2) continuous operation, and (3) semi-continuous (fed-batch) operation. A batch process is characterized by inoculation of the sterile culture medium (batch medium) with microorganisms at the start of the process, cultivated for a specific reaction period. During cultivation, cell concentrations, substrate concentrations (carbon source, nutrient salts, vitamins, etc.) and product concentrations change. Good mixing ensures that there are no significant local differences in composition or temperature of the reaction mixture. The reaction is non-stationary and cells are grown until the growth-limiting substrate (generally the carbon source) has been consumed.

Continuous operation is characterized in that fresh culture medium (feed medium) is added continuously to the fermenter and spent media and cells are drawn continuously from the fermenter at the same rate. In a continuous operation, growth rate is determined by the rate of medium addition, and the growth yield is determined by the concentration of the growth limiting substrate (e.g., carbon source). All reaction variables and control parameters remain constant in time and therefore a time-constant state is established in the fermenter followed by constant productivity and output.

Semi-continuous operation can be regarded as a combination of batch and continuous operation. The fermentation is started off as a batch process and when the growth-limiting substrate has been consumed, a continuous feed medium containing glucose and minerals is added in a specified manner (fed-batch). In other words, this operation employs both a batch medium and a feed medium to achieve cell growth and efficient production of the desired protein. No cells are added or taken away during the cultivation period and therefore the fermenter operates batchwise as far as the microorganisms are concerned. While the present disclosure can be utilized in a variety of processes, including those mentioned above, a particular utilization is in conjunction with a fed-batch process.

In each of the above processes, cell growth and product accumulation can be monitored indirectly by taking advantage of a correlation between metabolite formation and some other variable, such as medium pH, optical density, color, and titratable acidity. For example, optical density provides an indication of the accumulation of insoluble cell particles and can be monitored on-stream using a micro-OD unit coupled to a display device or a recorder, or off-line by sampling. Optical density readings at 600 nanometers (0D600) are used as a means of determining dry cell weight.

High-cell-density fermentations are generally described as those processes which result in a yield of >30 g cell dry weight/liter (0D600 >60) at a minimum, and in certain embodiments result in a yield of >40 g cell dry weight/liter (0D600 >80). All high-cell-density fermentation processes employ a concentrated nutrient media that is gradually metered into the fermenter in a “fed-batch” process. A concentrated nutrient feed media is utilized for high-cell-density processes in order to minimize the dilution of the fermenter contents during feeding. A fed-batch process is utilized because it allows the operator to control the carbon source feeding, which is important because if the cells are exposed to concentrations of the carbon source high enough to generate high cell densities, the cells will produce so much of the inhibitory biproduct, acetate, that growth will stop (Majewski and Domach, “Simple constrained-optimization view of acetate overflow in E. coli,” Biotechnol Bioeng 1990 Mar. 25; 35(7): 732-738).

Acetic acid and its deprotonated ion, acetate, together represent one of the main inhibitory byproducts of bacterial growth and recombinant protein production in bioreactors. At pH 7, acetate is the most prevalent form of acetic acid. Any excess carbon energy source may be converted to acetic acid when the amount of the carbon energy source greatly exceeds the processing ability of the bacterium. Research has shown that saturation of the tricarboxylic acid cycle and/or the electron transport chain is the most likely cause of the acetic acid accumulation. The choice of growth medium may affect the level of acetic acid inhibition; cells grown in defined media may be affected by acetic acid more than those grown in complex media. Replacement of glucose with glycerol may also greatly decrease the amount of acetic acid produced. It is believed that glycerol produces less acetic acid than glucose because its rate of transport into a cell is much slower than that of glucose. However, glycerol is more expensive than glucose, and may cause the bacteria to grow more slowly. The use of reduced growth temperatures can also decrease the speed of carbon source uptake and growth rate thus decreasing the production of acetic acid. Bacteria produce acetic acid not only in the presence of an excess carbon energy source or during fast growth, but also under anaerobic conditions. When bacteria such as E. coli are allowed to grow too fast, they may exceed the oxygen delivery ability of the bioreactor system which may lead to anaerobic growth conditions. To prevent this from happening, a slower constant growth rate may be maintained through nutrient limitation. Other methods for reducing acetic acid accumulation include genetic modification to prevent acetic acid production, addition of acetic acid utilization genes, and selection of strains with reduced acetic acid. E. coli BL21(DE3) is one of the strains that has been shown to produce lower levels of acetic acid because of its ability to use acetic acid in its glyoxylate shunt pathway.

Various larger-scale fed-batch fermenters are available for production of recombinant proteins. Larger fermenters have at least 1000 liters of capacity, preferably about 1000 to 100,000 liters of capacity (working volume), leaving adequate room for headspace. These fermenters use agitator impellers or other suitable means to distribute oxygen and nutrients, especially glucose (the preferred carbon/energy source). Small-scale fermentation refers generally to fermentation in a fermenter that is no more than approximately 100 liters in volumetric capacity, and in some specific embodiments no more than approximately 10 liters.

Standard reaction conditions for the fermentation processes used to produce recombinant proteins generally involve maintenance of pH at about 5.0 to 8.0 and cultivation temperatures ranging from 20 to 50 degrees C. for microbial host cells such as E. coli. In one embodiment, which utilizes E. coli as the host system, fermentation is performed at an optimal pH of about 7.0 and an optimal cultivation temperature of about 30 degrees C.

The standard nutrient media components in these fermentation processes generally include a source of energy, carbon, nitrogen, phosphorus, magnesium, and trace amounts of iron and calcium. In addition, the media may contain growth factors (such as vitamins and amino acids), inorganic salts, and any other precursors essential to product formation. The media may contain a transportable organophosphate such as a glycerophosphate, for example an alpha-glycerophosphate and/or a beta-glycerophosphate, and as a more specific example, glycerol-2-phosphate and/or glycerol-3-phosphate. The elemental composition of the host cell being cultivated can be used to calculate the proportion of each component required to support cell growth. The component concentrations will vary depending upon whether the process is a low-cell-density or a high-cell-density process. For example, the glucose concentrations in low-cell-density batch fermentation processes range from 1 to 5 g/L, while high-cell-density batch processes use glucose concentrations ranging from 45 g/L to 75 g/L. In addition, growth media may contain modest concentrations (for example, in the range of 0.1-5 mM, or 0.25 mM, 0.5 mM, 1 mM, 1.5 mM, or 2 mM) of protective osmolytes such as betaine, dimethylsulfoniopropionate, and/or choline.

One or more inducers can be introduced into the growth medium to induce expression of the gene product(s) of interest. Induction can be initiated during the exponential growth phase, for example, such as toward the end of the exponential growth phase but before the culture reaches maximum cell density, or at earlier or later times during fermentation. When expressing the gene product(s) of interest from one or more promoters inducible by depletion of nutrients such as phosphate, induction will occur when that nutrient has been sufficiently depleted from the growth medium, without the addition of an exogenous inducer.

During exponential growth of host cells, the metabolic rate is directly proportional to availability of oxygen and a carbon/energy source; thus, reducing the levels of available oxygen or carbon/energy sources, or both, will reduce metabolic rate. Manipulation of fermenter operating parameters, such as agitation rate or back pressure, or reducing O₂ pressure, modulates available oxygen levels and can reduce host cell metabolic rate. Reducing concentration or delivery rate, or both, of the carbon/energy source(s) has a similar effect. Furthermore, depending on the nature of the expression system, induction of expression can lead to a decrease in host cell metabolic rate. Finally, upon reaching maximum cell density, the growth rate stops or decreases dramatically. Reduction in host cell metabolic rate can result in more controlled expression of the gene product(s) of interest, including the processes of protein folding and assembly. Host cell metabolic rate can be assessed by measuring cell growth rates, either specific growth rates or instantaneous growth rates (by measuring optical density (OD) such as OD₆₀₀ and/or optionally by converting OD to biomass). The approximate biomass (cell dry weight) at each assayed point is calculated: approximate biomass (g)=(OD₆₀₀÷2)×volume (L). Desirable growth rates are, in certain embodiments, in the range of 0.01 to 0.7, or are in the range of 0.05 to 0.3, or are in the range of 0.1 to 0.2, or are approximately 0.15 (0.15 plus-or-minus 10%), or are 0.15.

Fermentation Equipment. The following are examples of equipment that can be used to grow host cells; many other configurations of fermentation systems are commercially available. Host cells can be grown in a New Brunswick BioFlo/CelliGen 115 water jacketed fermenter (Eppendorf North America, Hauppauge, N.Y.), 1 L vessel size with a 2× Rushton impeller and a BioFlo/CelliGen 115 Fermenter/Bioreactor controller; temperature, pH, and dissolved oxygen (DO) are monitored. It is also possible to grow host cells in a four-fold configurable DASGIP system (Eppendorf North America, Hauppauge, N.Y.) comprising four 60- to 250-ml DASbox fermentation vessels, each with a 2× Rushton impeller, a DASbox exhaust condenser, and a DASbox feeding and monitoring module (which includes a temperature sensor, a pH/redox sensor, and a dissolved oxygen sensor). Suitable fermentation equipment also includes NLF 22 30 L lab fermenters (Bioengineering, Inc., Somerville, Mass.), with 30-L capacity and 20-L maximum working volume in a stainless steel vessel; two Rushton impellers, sparged with air only; and a control system running BioSCADA software that allows for tracking and control of all relevant parameters including pH, DO, exhaust O₂, exhaust CO₂, temperature, and pressure.

Purification of intein fusion polypeptides and target polypeptides. Polypeptides that include a 6×His tag can be purified by immobilized metal affinity chromatography (IMAC), such as the use of a nickel-nitrilotriacetic acid (Ni-NTA) column to specifically retain the 6×His-tagged polypeptide of interest while other molecules flow through. IMAC exploits interactions between histidine residues and divalent metal ions, most commonly Ni²⁺; other metal ions including Cu²⁺, Co²⁺, Fe²⁺, and Zn²⁺ have also been shown to have affinity for His residues. The metal ions are typically immobilized on the matrix via various metal-chelator systems, including iminodiacetic acid (IDA) and the more commonly used nitrilotriacetic acid (NTA). A wide variety of matrices are commercially available such as nickel-nitrilotriacetic acid (Ni-NTA), Ni Sepharose, and copper-carboxylmethylaspartate (CO-CMA). The column can be equilibrated with a buffer such as 50 mM Tris, 3 M urea, 0.5 M NaCl, 25 mM imidazole, pH 8.0. After binding of the 6×HIs-tagged polypeptide, a wash step with a buffer containing a low concentration of imidazole (0 mM, or 10 to 50 mM), or a buffer with a pH higher or lower than that of the binding buffer, can be included to remove nonspecific proteins that are weakly bound to the column during sample loading. For example, a wash buffer of 50 mM Tris, 100 mM NaCl, pH 10 can be used. The 6×His-tagged polypeptide can be eluted from the matrix using a buffer containing imidazole at a concentration of at least 100 mM imidazole, or 250 to 500 mM imidazole, or 500 mM imidazole. It is also possible to elute the polypeptide(s) of interest by lowering the buffer pH, and/or by including chelating agents such as EDTA (at a concentration of 50 to 200 mM, or 100 mM) in the elution buffer. For example, an elution buffer of 50 mM Tris, 100 mM NaCl, 100 mM imidazole, pH 10 can be used. Purification methods for gene products that include a polyhistidine tag are further described in Bornhorst and Falke, “Purification of proteins using polyhistidine affinity tags,” Methods Enzymol 2000; 326: 245-254, which is incorporated by reference herein. In the purification by IMAC of 6×His-tagged CPBpro proinsulin proteins from solubilizable complexes, using either Ni-NTA Superflow (QIAgen, Germantown, Md.) or HisTrap HP Ni Sepharose columns (GE Healthcare, Pittsburgh, Pa.), this method allowed for purification of the proinsulin gene product to greater than 90% purity.

For samples lacking a 6×His tag, or for procedures where use of such a tag is not necessary, cation or anion exchange chromatography, such as the use of DEAE resins, and/or reversed-phase or high-performance liquid chromatography (RPLC or HPLC), can be employed to further separate the polypeptide of interest from other contaminants or from the unwanted product(s) of chemical or enzymatic treatment.

### Example 1

### Use of Intein Polypeptides to Produce TRAST-Fab with Native N-Termini

A. Expression of Intein-TRAST-Fab Heterodimers in Host Cells

Trastuzumab, also referred to by its trade name Herceptin®, is a full-length humanized IgG1 monoclonal antibody that recognizes the HER2 antigen, also called ERBB2. TRAST-Fab is an antigen-binding fragment of trastuzumab; the amino acid sequences of a TRAST-Fab heavy chain (‘HC’) and a TRAST-Fab light chain (‘LC’) are presented in SEQ ID NOs: 22 and 23, respectively. Within the TRAST Fab heterodimer, the heavy chain and light chain are connected by an intermolecular disulfide bond (in addition to intramolecular disulfide bonds within each of the chains).

Expression constructs were created in which the DNA sequences encoding the TRAST-Fab heavy chain and the TRAST-Fab light chain (SEQ ID NOs: 22 and 23, respectively) each had an intein-encoding DNA sequence added at their 5′ ends, with the intein amino acid sequences for each type of expression construct being one of SEQ ID NOs: 1-6 as described in Table 1. The intein-TRAST-Fab-encoding DNA sequences were cloned into a dual-promoter vector downstream of an L-arabinose-inducible promoter (ParaBAD or ‘ara promoter’) in a bicistronic arrangement with the sequence encoding intein-TRAST-HC placed upstream of the sequence encoding intein-TRAST-LC.

Because there were two polypeptides each comprising an intein at their N-terminus to be expressed in E. coli from a single expression vector, two different DNA sequences encoding each intein were prepared—intein-encoding sequence #1 and intein-encoding sequence #2—that had optimized codon usage for E. coli, and that were different enough to reduce the possibility of a recombination event between them. The two intein-encoding sequences for each of the inteins having SEQ ID NOs: 1-9 are listed in Table 1 and have SEQ ID NOs: 24-41.

Expression constructs were also created in a similar way for the expression of DnaX(split_C)-TRAST-Fab and DnaB(split_C)-TRAST-Fab, in the presence of DnaX(split_N) or DnaB(split_N), respectively. A DNA sequence encoding the 6×His-tagged C-terminal portion of the split intein (SEQ ID NO: 12 for DnaX(split_C) and SEQ ID NO: 15 for DnaB(split_C)) was placed upstream of each of the sequences encoding TRAST-Fab HC and TRAST-Fab LC, in a bicistronic arrangement downstream of the ara promoter. These constructs were dual-promoter expression constructs also comprising a propionate-inducible PprpBCDE or ‘prp promoter’, and the prp promoter was used to express the N-terminal portion of the split intein (SEQ ID NO: 10 for DnaX(split_N) and SEQ ID NO: 13 for DnaB(split_N)). In the same manner as for the intact inteins, two different DNA sequences were used to encode the two C-terminal portions of the split intein present in each expression construct, as shown in Table 1.

E. coli 521 cells were transformed with different expression vectors expressing intein-TRAST-Fab heterodimers, each comprising inteins having one of the amino acid sequences of SEQ ID NOs: 1-5, described in Table 1. Following transformation with the expression vector(s), the host cell samples were plated onto solid media containing kanamycin to select for successful transformants comprising expression vectors, which carry a gene for kanamycin resistance. The host cells were cultured, and expression of the intein-TRAST-Fab was induced with L-arabinose.

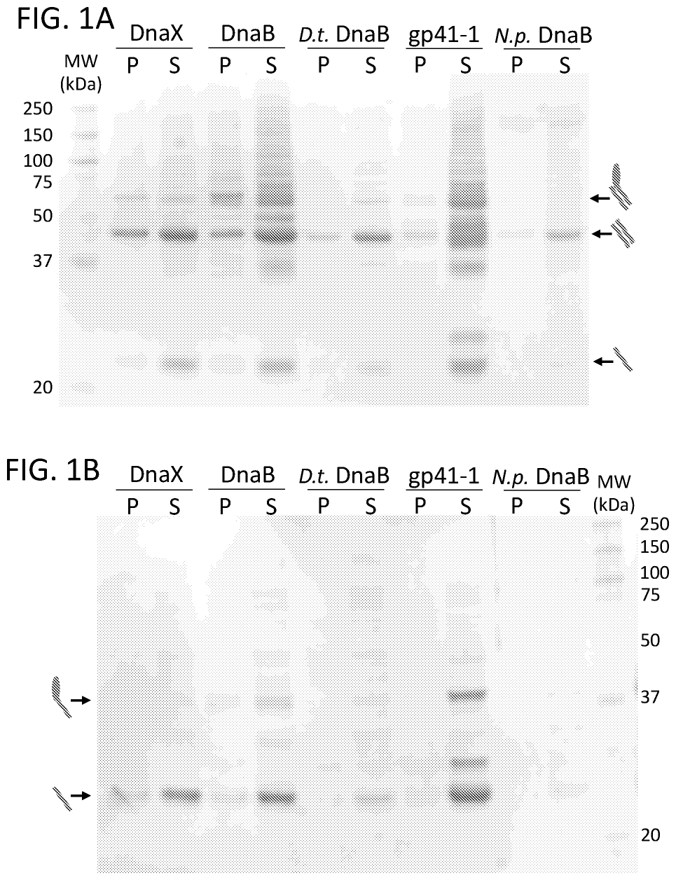

The host cells with the expression vectors expressing DnaX-TRAST-Fab, DnaB-TRAST-Fab, D.t. DnaB-TRAST-Fab, gp41-1-TRAST-Fab, and N.p. DnaB-TRAST-Fab, respectively, were harvested following induction, and lysed at pH 7.4. Duplicate samples of host cells expressing DnaB-TRAST-Fab were lysed at pH 6.4 and at pH 8.0. Lysis under all three pH conditions produced comparable results. The host cell lysates were subjected to centrifugation, the soluble fraction was collected from each sample, and each pellet was resuspended. The soluble fractions (‘S’) and the resuspended pellets (‘P’) were subjected to polyacrylamide gel electrophoresis under non-reducing conditions and also under reducing conditions. The proteins in the gels were then transferred to a membrane, contacted with a primary antibody that binds to human IgG, and detected using a secondary antibody. The resulting Western blots are shown in FIGS. 1A and 1B. For each of the different intein-TRAST-Fab samples, it can be seen that the intein has been excised from the majority of the TRAST-Fab polypeptide species detected on the Western blots.

E. coli 521 cells were also transformed with the expression constructs encoding DnaX(split_C)-TRAST-Fab/DnaX(split_N) and DnaB(split_C)-TRAST-Fab/DnaB(split_N); the host cells were grown in media containing kanamycin, and expression of the intein(split_C)-TRAST-Fab polypeptides was induced with L-arabinose, and expression of intein(split_N) was induced with propionate. The induced host cells were harvested, lysed, and the lysates analyzed by polyacrylamide gel electrophoresis. Coexpression of the intein(split_C)-TRAST-Fab polypeptides and the intein(split_N) polypeptide resulted in cleavage of the intein(split_C) from the N-terminal end of the TRAST-Fab polypeptides.

B. LC-MS Analysis of Intein-TRAST-Fab Heterodimers

E. coli 521 host cells comprising expression vectors expressing DnaX-TRAST-Fab, DnaB-TRAST-Fab, D.t. DnaB-TRAST-Fab, and gp41-1-TRAST-Fab were cultured in 24-well plates, along with E. coli 521 host cells expressing a control Met-TRAST-Fab heterodimer from coding sequences downstream of the ara promoter in a bicistronic arrangement, with the sequence encoding Met-TRAST-LC in this construct placed upstream of the sequence encoding Met-TRAST-HC. Expression of the control Met-TRAST-Fab and intein-TRAST-Fab was induced in growth medium that included 50 micrograms/mL kanamycin and L-arabinose at varying concentrations: 0.0013 mM, 0.0067 mM, 0.0333 mM, 0.1667 mM, 0.8333 mM, and 4.1667 mM. Expression of Met-TRAST-Fab and intein-TRAST-Fab was induced for 16 hours, and then three samples were collected from each well.

The Met-TRAST-Fab and intein-TRAST-Fab samples were lysed in lysis buffer (50 mM Tris pH 7.4, 200 mM NaCl, comprising protease inhibitors, benzonase, and lysozyme), then purified by Protein L affinity capture. Liquid chromatography mass spectrometry (LC-MS) analysis was performed by running the samples over a ZORBAX RRHD 300A StableBond diphenyl HPLC column (Agilent Technologies, Santa Clara, Calif.), with a gradient elution from 28 to 38% solvent B (0.085% TFA in acetonitrile), at a constant temperature of 70 degrees C. Elutions from the column were detected both by UV at 214 nm and 280 nm and by MS1 scans from 600-3000 m/z on a 5600 quadrupole time-of-flight (QTOF) mass spectrometer (SCIEX, Framingham, Mass.). Mass/charge (m/z) ranges pertaining to TRAST-Fab polypeptides having native N-terminal amino acids were selected for post-data-acquisition ion extraction to evaluate the generation of native N-termini by intein-mediated cleavage of the intein-TRAST-Fab polypeptides.

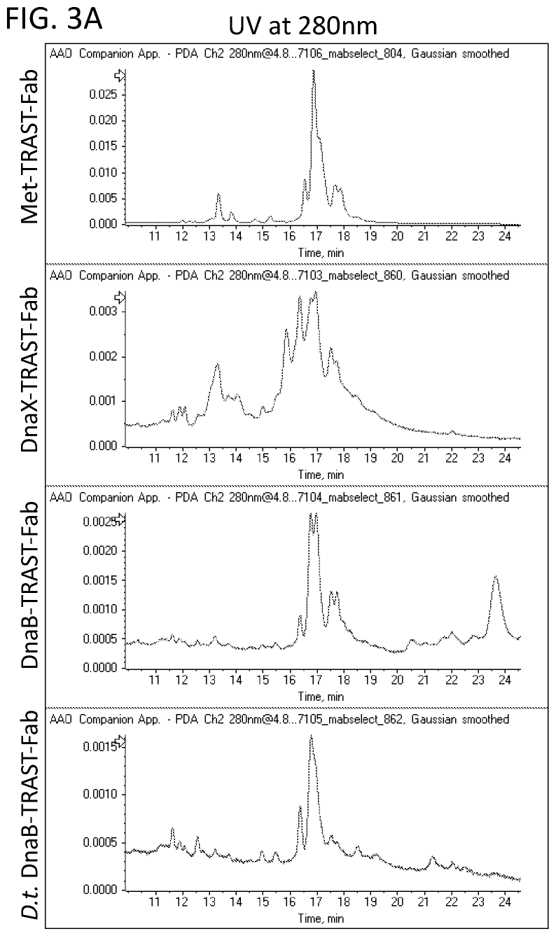

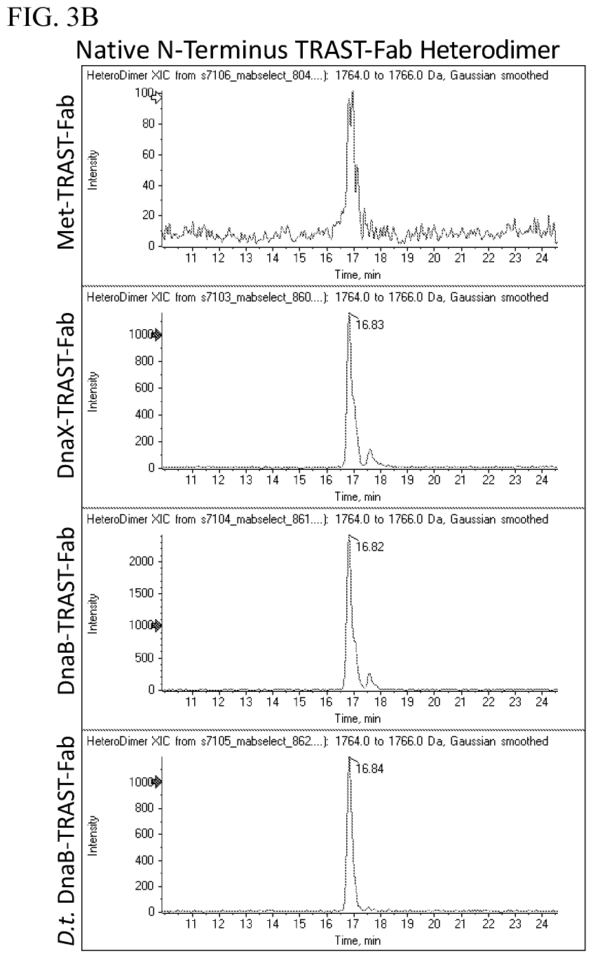

The results of the LC-MS analysis are provided in FIGS. 2 and 3. FIG. 2 shows the result of samples analyzed under reducing conditions, with panels 2B and 2C showing the intensity of peaks corresponding to the native N-termini of the light chain and of the heavy chain, respectively. As can be seen from FIGS. 2A-2C, the control Met-TRAST-Fab produced very low amounts of light chain or heavy chain with native N-termini (i.e., without an N-terminal Met residue), but DnaB-TRAST-Fab and gp41-1-TRAST-Fab did produce significant amounts of TRAST-Fab polypeptides with native N-termini. In Panel B of FIG. 3, analyzed in non-reducing conditions, the amount of TRAST-Fab heterodimer with native N-termini is compared between the Met-TRAST-Fab control, DnaX-TRAST-Fab, DnaB-TRAST-Fab, and D.t. DnaB-TRAST-Fab. Although in these chromatographs it is possible to see the peak produced by the removal of Met residues from the Met-TRAST-Fab control to generate native N-termini (peak intensity of about 100), the amount of native N-termini TRAST-Fab produced by each of the intein-containing constructs was much higher (peak intensities of 1000-2000).

### Example 2

### Polynucleotides Encoding High-Performing Intein-Fusion Polypeptides

As listed in Table 1 and described in Example 1A above, two different polynucleotides that encode the same intein amino acid sequence were prepared for many of the intein polypeptides listed in Table 1. These intein-coding sequences were codon-optimized for improved expression in E. coli. Experiments were performed to identify polynucleotides, which encoded the DnaB (SEQ ID NO: 2) amino acid sequence portion of DnaB-fusion polypeptides, that were useful in the expression of high levels of active protein of interest in an E. coli host cell. A library of expression vectors was constructed in which the two polypeptide chains of the protein of interest were expressed in a bicistronic arrangement in the same manner as described in Example 1A for the TRAST-Fab HC and LC; each polypeptide chain was expressed as a DnaB-fusion polypeptide with DnaB at the N-terminus of the fusion polypeptide. The DnaB portion of each fusion polypeptide was encoded by a different polynucleotide sequence, as described in Example 1A with respect to SEQ ID NOs: 26 and 27. A subset of the expression vectors in the library varied at several positions within the DnaB-encoding polynucleotides of SEQ ID NOs: 26 and 27, between nucleotide positions 9 and 30 of those sequences, to produce many different silent changes in the third through tenth codons of each DnaB coding sequence.

The expression vector library encoding the DnaB-fusion polypeptides was transformed into E. coli host cells, the host cells were cultured, and expression of the DnaB-fusion polypeptides was induced with L-arabinose. The host cells were assessed for the level of expression of active protein of interest formed from the DnaB-fusion polypeptides, and a subpopulation of high-expressing host cells was selected. The expression vectors present in the subpopulation of high-expressing host cells were sequenced by next-generation sequencing (NGS), and the relative proportions of different DnaB-encoding polynucleotides present within the subpopulation of high-expressing host cells were determined. At each of the two DnaB-coding positions within the expression vectors, the polynucleotide sequences that were enriched to the greatest degree, when comparing the starting population of host cells to the high-expressing host cell subpopulation, were the DnaB-coding sequences of SEQ ID NOs: 26 and 27. This result indicates that the codon-optimized DnaB-coding sequences of SEQ ID NOs: 26 and 27 had the highest average degree of correlation with the high-expression characteristic of the selected host cells. None of the silent coding sequence changes in the third through tenth codons of each DnaB coding sequence resulted in a DnaB-coding sequence that was enriched to a greater degree than SEQ ID NOs: 26 and 27 when selecting for a high level of expression of active protein of interest across the population of host cells.

A further experiment was performed using the expression vector library, which encoded DnaB-fusion polypeptides with silent coding sequence changes in the third through tenth codons of each DnaB coding sequence, as described above. In this experiment, host cells evidencing a high level of expression of active protein of interest were selected, and further rounds of selection of host cells were performed using specific activity assays on the protein of interest produced, and assays of the amount of structurally correct protein of interest produced, such as solid-phase extraction mass spectrometry (SPE-MS) assays. Based on these criteria, high-performing host cells were selected and the expression vectors they contain were sequenced. Four additional DnaB-coding sequences were identified in this way that demonstrated improvement in expression of active protein of interest when compared to the original expression vector comprising SEQ ID NOs: 26 and 27; the four DnaB-coding sequences are all variations of SEQ ID NO: 27 and are presented as SEQ ID NOs: 61-64.

### Example 3

### Determination of Polynucleotide or Amino Acid Sequence Similarity

Percent polynucleotide sequence or amino acid sequence identity is defined as the number of aligned symbols (e.g., nucleotides or amino acids), that are identical in both aligned sequences, divided by the total number of symbols in the alignment of the two sequences, including gaps. The degree of similarity (percent identity) between two sequences may be determined by aligning the sequences using the global alignment method of Needleman and Wunsch (J. Mol. Biol. 48:443, 1970), as implemented by the National Center for Biotechnology Information (NCBI) in the Needleman-Wunsch Global Sequence Alignment Tool, available through the website blast.ncbi.nlm.nih.gov/Blast.cgi. In one embodiment, the Needleman and Wunsch alignment parameters are set to the default values (Match/Mismatch Scores of 2 and −3, respectively, and Gap Costs for Existence and Extension of 5 and 2, respectively). Other programs used by those skilled in the art of sequence comparison may also be used to align sequences, such as, for example, the basic local alignment search tool or BLAST® program (Altschul et al., “Basic local alignment search tool,” J Mol Biol 1990 Oct. 5; 215(3): 403-410), as implemented by NCBI at the blast.ncbi.nlm.nih.gov/Blast.cgi website, using the default parameter settings. The BLAST algorithm has multiple optional parameters including two that may be used as follows: (A) inclusion of a filter to mask segments of the query sequence that have low compositional complexity or segments consisting of short-periodicity internal repeats, which is preferably not utilized or set to ‘off’, and (B) a statistical significance threshold for reporting matches against database sequences, called the ‘Expect’ or E-score (the expected probability of matches being found merely by chance; if the statistical significance ascribed to a match is greater than this E-score threshold, the match will not be reported). If this ‘Expect’ or E-score value is adjusted from the default value (10), preferred threshold values are 0.5, or in order of increasing preference, 0.25, 0.1, 0.05, 0.01, 0.001, 0.0001, 0.00001, and 0.000001.

In practicing the present invention, many conventional techniques in molecular biology, microbiology, and recombinant DNA technology are optionally used. Such conventional techniques relate to vectors, host cells, and recombinant methods. These techniques are well known and are explained in, for example, Berger and Kimmel, Guide to Molecular Cloning Techniques, Methods in Enzymology, Volume 152 Academic Press, Mc, San Diego, Calif.; Sambrook et al., Molecular Cloning—A Laboratory Manual (3rd Ed.), Vol. 1-3, Cold Spring Harbor Laboratory, Cold Spring Harbor, N.Y., 2000; and Current Protocols in Molecular Biology, F. M. Ausubel et al., eds., Current Protocols, a joint venture between Greene Publishing Associates, Inc. and John Wiley & Sons, Inc., (supplemented through 2006). Other useful references, for example for cell isolation and culture and for subsequent nucleic acid or protein isolation, include Freshney (1994) Culture of Animal Cells, A Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg N.Y.); and Atlas and Parks (Eds.) The Handbook of Microbiological Media (1993) CRC Press, Boca Raton, Fla. Methods of making nucleic acids (for example, by in vitro amplification, purification from cells, or chemical synthesis), methods for manipulating nucleic acids (for example, by site-directed mutagenesis, restriction enzyme digestion, ligation, etc.), and various vectors, cell lines, and the like useful in manipulating and making nucleic acids are described in the above references. In addition, essentially any polynucleotide (including labeled or biotinylated polynucleotides) can be custom or standard ordered from any of a variety of commercial sources.

The present invention has been described in terms of particular embodiments found or proposed to comprise certain modes for the practice of the invention. It will be appreciated by those of ordinary skill in the art that, in light of the present disclosure, numerous modifications and changes can be made in the particular embodiments exemplified without departing from the intended scope of the invention. Any embodiments or features of embodiments can be combined with one another, and such combinations are expressly encompassed within the scope of the present invention.

All cited references, including patent publications, are incorporated herein by reference in their entirety. Nucleotide and other genetic sequences, referred to by published genomic location or other description, are also expressly incorporated herein by reference.

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

### Sequences Presented in the Sequence Listing

| SEQ | | | | |
|---|---|---|---|---|
| ID NO: | Length: | Type: | Organism: | Description; ‘Other Information’ |
| 1 | 151 | PRT | Artificial | Synechocystis sp. PCC6803 DnaX 6xHis-tagged |
| | | | Sequence | non-ligating mini-intein |
| 2 | 169 | PRT | Artificial | Synechocystis sp. PCC6803 DnaB 6xHis-tagged |
| | | | Sequence | non-ligating mini-intein |
| 3 | 140 | PRT | Artificial | Desulfofundulus thermosubterraneus DSM |
| | | | Sequence | 16057 DnaB non-ligating intein |
| 4 | 136 | PRT | Artificial | Prochlorococcus cyanophage P-SSM2 gp41-1 |
| | | | Sequence | 6xHis-tagged non-ligating mini-intein |
| 5 | 140 | PRT | Artificial | Nostoc punctiforme strain ATCC 29133/PCC |
| | | | Sequence | 73102 DnaB non-ligating mini-intein |
| 6 | 170 | PRT | Artificial | Mycobacterium tuberculosis strain ATCC |
| | | | Sequence | 25618/H37Rv RecA non-ligating mini-intein |
| 7 | 136 | PRT | Artificial | Clostridium thermocellum CthBIL4 N115D |
| | | | Sequence | non-ligating intein |
| 8 | 168 | PRT | Artificial | Deinococcus swuensis PolIII subunit alpha non- |
| | | | Sequence | ligating mini-intein |
| 9 | 163 | PRT | Artificial | Cyanobacterium aponinum DnaB non-ligating |
| | | | Sequence | intein |
| 10 | 95 | PRT | Artificial | Synechocystis sp. PCC6803 DnaX non-ligating |
| | | | Sequence | split intein, N-terminal portion |
| 11 | 43 | PRT | Artificial | Synechocystis sp. PCC6803 DnaX non-ligating |
| | | | Sequence | split intein, C-terminal portion |
| 12 | 61 | PRT | Artificial | Synechocystis sp. PCC6803 DnaX 6xHis-tagged |
| | | | Sequence | non-ligating split intein, C-terminal portion |
| 13 | 107 | PRT | Artificial | Synechocystis sp. PCC6803 DnaB non-ligating |
| | | | Sequence | split intein, N-terminal portion |
| 14 | 49 | PRT | Artificial | Synechocystis sp. PCC6803 DnaB non-ligating |
| | | | Sequence | split intein, C-terminal portion |
| 15 | 67 | PRT | Artificial | Synechocystis sp. PCC6803 DnaB 6xHis-tagged |
| | | | Sequence | non-ligating split intein, C-terminal portion |
| 16 | 89 | PRT | Artificial | Prochlorococcus cyanophage P-SSM2 gp41-1 |
| | | | Sequence | non-ligating split intein, N-terminal portion |
| 17 | 36 | PRT | Artificial | Prochlorococcus cyanophage P-SSM2 gp41-1 |
| | | | Sequence | non-ligating split intein, C-terminal portion |
| 18 | 94 | PRT | Artificial | Clostridium thermocellum CthBIL4 N115D |
| | | | Sequence | non-ligating split intein ‘C42’, N-terminal |
| | | | | portion |
| 19 | 43 | PRT | Artificial | Clostridium thermocellum CthBIL4 N115D |
| | | | Sequence | non-ligating split intein ‘C42’, C-terminal |
| | | | | portion |
| 20 | 120 | PRT | Artificial | Clostridium thermocellum CthBIL4 N115D |
| | | | Sequence | non-ligating split intein ‘C16’, N-terminal |
| | | | | portion |
| 21 | 17 | PRT | Artificial | Clostridium thermocellum CthBIL4 N115D |
| | | | Sequence | non-ligating split intein ‘C16’, C-terminal |
| | | | | portion |
| 22 | 227 | PRT | Artificial | TRAST-Fab heavy chain |
| | | | Sequence | |
| 23 | 214 | PRT | Artificial | TRAST-Fab light chain |
| | | | Sequence | |
| 24 | 453 | DNA | Artificial | Codon-optimized sequence #1 encoding |
| | | | Sequence | Synechocystis sp. PCC6803 DnaX 6xHis-tagged |
| | | | | non-ligating mini-intein (SEQ ID NO: 1) |
| 25 | 453 | DNA | Artificial | Codon-optimized sequence #2 encoding |
| | | | Sequence | Synechocystis sp. PCC6803 DnaX 6xHis-tagged |
| | | | | non-ligating mini-intein (SEQ ID NO: 1) |
| 26 | 507 | DNA | Artificial | Codon-optimized sequence #1 encoding |
| | | | Sequence | Synechocystis sp. PCC6803 DnaB 6xHis-tagged |
| | | | | non-ligating mini-intein (SEQ ID NO: 2) |
| 27 | 507 | DNA | Artificial | Codon-optimized sequence #2 encoding |
| | | | Sequence | Synechocystis sp. PCC6803 DnaB 6xHis-tagged |
| | | | | non-ligating mini-intein (SEQ ID NO: 2) |
| 28 | 420 | DNA | Artificial | Codon-optimized sequence #1 encoding |
| | | | Sequence | Desulfofundulus thermosubterraneus DSM |
| | | | | 16057 DnaB non-ligating intein (SEQ ID NO: 3) |
| 29 | 420 | DNA | Artificial | Codon-optimized sequence #2 encoding |
| | | | Sequence | Desulfofundulus thermosubterraneus DSM |
| | | | | 16057 DnaB non-ligating intein (SEQ ID NO: 3) |
| 30 | 408 | DNA | Artificial | Codon-optimized sequence #1 encoding |
| | | | Sequence | Prochlorococcus cyanophage P-SSM2 gp41-1 |
| | | | | 6xHis-tagged non-ligating mini-intein (SEQ ID |
| | | | | NO: 4) |
| 31 | 408 | DNA | Artificial | Codon-optimized sequence #2 encoding |
| | | | Sequence | Prochlorococcus cyanophage P-SSM2 gp41-1 |
| | | | | 6xHis-tagged non-ligating mini-intein (SEQ ID |
| | | | | NO: 4) |
| 32 | 420 | DNA | Artificial | Codon-optimized sequence #1 encoding Nostoc |
| | | | Sequence | punctiforme strain ATCC 29133/PCC 73102 |
| | | | | DnaB non-ligating mini-intein (SEQ ID NO: 5) |
| 33 | 420 | DNA | Artificial | Codon-optimized sequence #2 encoding Nostoc |
| | | | Sequence | punctiforme strain ATCC 29133/PCC 73102 |
| | | | | DnaB non-ligating mini-intein (SEQ ID NO: 5) |
| 34 | 510 | DNA | Artificial | Codon-optimized sequence #1 encoding |
| | | | Sequence | Mycobacterium tuberculosis strain ATCC |
| | | | | 25618/H37Rv RecA non-ligating mini-intein |
| | | | | (SEQ ID NO: 6) |
| 35 | 510 | DNA | Artificial | Codon-optimized sequence #2 encoding |
| | | | Sequence | Mycobacterium tuberculosis strain ATCC |
| | | | | 25618/H37Rv RecA non-ligating mini-intein |
| | | | | (SEQ ID NO: 6) |
| 36 | 408 | DNA | Artificial | Codon-optimized sequence #1 encoding |
| | | | Sequence | Clostridium thermocellum CthBIL4 N115D |
| | | | | engineered BIL (bacterial non-ligating intein- |
| | | | | like) (SEQ ID NO: 7) |
| 37 | 408 | DNA | Artificial | Codon-optimized sequence #2 encoding |
| | | | Sequence | Clostridium thermocellum CthBIL4 N115D |
| | | | | engineered BIL (bacterial non-ligating intein- |
| | | | | like) (SEQ ID NO: 7) |
| 38 | 504 | DNA | Artificial | Codon-optimized sequence #1 encoding |
| | | | Sequence | Deinococcus swuensis PolIII alpha subunit non- |
| | | | | ligating mini-intein (SEQ ID NO: 8) |
| 39 | 504 | DNA | Artificial | Codon-optimized sequence #2 encoding |
| | | | Sequence | Deinococcus swuensis PolIII alpha subunit non- |
| | | | | ligating mini-intein (SEQ ID NO: 8) |
| 40 | 489 | DNA | Artificial | Codon-optimized sequence #1 encoding |
| | | | Sequence | Cyanobacterium aponinum DnaB non-ligating |
| | | | | intein (SEQ ID NO: 9) |
| 41 | 489 | DNA | Artificial | Codon-optimized sequence #2 encoding |
| | | | Sequence | Cyanobacterium aponinum DnaB non-ligating |
| | | | | intein (SEQ ID NO: 9) |
| 42 | 285 | DNA | Artificial | Codon-optimized sequence encoding |
| | | | Sequence | Synechocystis sp. PCC6803 DnaX non-ligating |
| | | | | split intein, N-terminal portion (SEQ ID NO: 10) |
| 43 | 129 | DNA | Artificial | Codon-optimized sequence #1 encoding |
| | | | Sequence | Synechocystis sp. PCC6803 DnaX non-ligating |
| | | | | split intein, C-terminal portion (SEQ ID NO: 11) |
| 44 | 129 | DNA | Artificial | Codon-optimized sequence #2 encoding |
| | | | Sequence | Synechocystis sp. PCC6803 DnaX non-ligating |
| | | | | split intein, C-terminal portion (SEQ ID NO: 11) |
| 45 | 183 | DNA | Artificial | Codon-optimized sequence #1 encoding |
| | | | Sequence | Synechocystis sp. PCC6803 DnaX 6xHis-tagged |
| | | | | non-ligating split intein, C-terminal portion |
| | | | | (SEQ ID NO: 12) |
| 46 | 183 | DNA | Artificial | Codon-optimized sequence #2 encoding |
| | | | Sequence | Synechocystis sp. PCC6803 DnaX 6xHis-tagged |
| | | | | non-ligating split intein, C-terminal portion |
| | | | | (SEQ ID NO: 12) |
| 47 | 321 | DNA | Artificial | Codon-optimized sequence encoding |
| | | | Sequence | Synechocystis sp. PCC6803 DnaB non-ligating |
| | | | | split intein, N-terminal portion (SEQ ID NO: 13) |
| 48 | 147 | DNA | Artificial | Codon-optimized sequence #1 encoding |
| | | | Sequence | Synechocystis sp. PCC6803 DnaB non-ligating |
| | | | | split intein, C-terminal portion (SEQ ID NO: 14) |
| 49 | 147 | DNA | Artificial | Codon-optimized sequence #2 encoding |
| | | | Sequence | Synechocystis sp. PCC6803 DnaB non-ligating |
| | | | | split intein, C-terminal portion (SEQ ID NO: 14) |
| 50 | 201 | DNA | Artificial | Codon-optimized sequence #1 encoding |
| | | | Sequence | Synechocystis sp. PCC6803 DnaB 6xHis-tagged |
| | | | | non-ligating split intein, C-terminal portion |
| | | | | (SEQ ID NO: 15) |
| 51 | 201 | DNA | Artificial | Codon-optimized sequence #2 encoding |
| | | | Sequence | Synechocystis sp. PCC6803 DnaB 6xHis-tagged |
| | | | | non-ligating split intein, C-terminal portion |
| | | | | (SEQ ID NO: 15) |
| 52 | 267 | DNA | Artificial | Codon-optimized sequence encoding |
| | | | Sequence | Prochlorococcus cyanophage P-SSM2 gp41-1 |
| | | | | non-ligating split intein, N-terminal portion |
| | | | | (SEQ ID NO: 16) |
| 53 | 108 | DNA | Artificial | Codon-optimized sequence #1 encoding |
| | | | Sequence | Prochlorococcus cyanophage P-SSM2 gp41-1 |
| | | | | non-ligating split intein, C-terminal portion |
| | | | | (SEQ ID NO: 17) |
| 54 | 108 | DNA | Artificial | Codon-optimized sequence #2 encoding |
| | | | Sequence | Prochlorococcus cyanophage P-SSM2 gp41-1 |
| | | | | non-ligating split intein, C-terminal portion |
| | | | | (SEQ ID NO: 17) |
| 55 | 282 | DNA | Artificial | Codon-optimized sequence encoding |
| | | | Sequence | Clostridium thermocellum CthBIL4 N115D |
| | | | | non-ligating split intein ‘C42’, N-terminal |
| | | | | portion (SEQ ID NO: 18) |
| 56 | 129 | DNA | Artificial | Codon-optimized sequence #1 encoding |
| | | | Sequence | Clostridium thermocellum CthBIL4 N115D |
| | | | | non-ligating split intein ‘C42’, C-terminal |
| | | | | portion (SEQ ID NO: 19) |
| 57 | 129 | DNA | Artificial | Codon-optimized sequence #2 encoding |
| | | | Sequence | Clostridium thermocellum CthBIL4 N115D |
| | | | | non-ligating split intein ‘C42’, C-terminal |
| | | | | portion (SEQ ID NO: 19) |
| 58 | 360 | DNA | Artificial | Codon-optimized sequence encoding |
| | | | Sequence | Clostridium thermocellum CthBIL4 N115D |
| | | | | non-ligating split intein ‘C16’, N-terminal |
| | | | | portion (SEQ ID NO: 20) |
| 59 | 51 | DNA | Artificial | Codon-optimized sequence #1 encoding |
| | | | Sequence | Clostridium thermocellum CthBIL4 N115D |
| | | | | non-ligating split intein 'Cl6', C-terminal |
| | | | | portion (SEQ ID NO: 21) |
| 60 | 51 | DNA | Artificial | Codon-optimized sequence #2 encoding |
| | | | Sequence | Clostridium thermocellum CthBIL4 N115D |
| | | | | non-ligating split intein ‘C16’, C-terminal |
| | | | | portion (SEQ ID NO: 21) |
| 61 | 507 | DNA | Artificial | Codon-optimized sequence #2A encoding |
| | | | Sequence | Synechocystis sp. PCC6803 DnaB 6xHis-tagged |
| | | | | non-ligating mini-intein (SEQ ID NO: 2) |
| 62 | 507 | DNA | Artificial | Codon-optimized sequence #2B encoding |
| | | | Sequence | Synechocystis sp. PCC6803 DnaB 6xHis-tagged |
| | | | | non-ligating mini-intein (SEQ ID NO: 2) |
| 63 | 507 | DNA | Artificial | Codon-optimized sequence #2C encoding |
| | | | Sequence | Synechocystis sp. PCC6803 DnaB 6xHis-tagged |
| | | | | non-ligating mini-intein (SEQ ID NO: 2) |
| 64 | 507 | DNA | Artificial | Codon-optimized sequence #2D encoding |
| | | | Sequence | Synechocystis sp. PCC6803 DnaB 6xHis-tagged |
| | | | | non-ligating mini-intein (SEQ ID NO: 2) |
| 65 | 507 | DNA | Artificial | Codon-optimized sequence DnaB_Alt_v02 |
| | | | Sequence | encoding Synechocystis sp. PCC6803 DnaB |
| | | | | 6xHis-tagged non-ligating mini-intein (SEQ ID |
| | | | | NO: 2) |
| 66 | 507 | DNA | Artificial | Codon-optimized sequence DnaB_Alt_v03 |
| | | | Sequence | encoding Synechocystis sp. PCC6803 DnaB |
| | | | | 6xHis-tagged non-ligating mini-intein (SEQ ID |
| | | | | NO: 2) |
| 67 | 507 | DNA | Artificial | Codon-optimized sequence DnaB_KA encoding |
| | | | Sequence | Synechocystis sp. PCC6803 DnaB 4xHisKA- |
| | | | | tagged non-ligating mini-intein (SEQ ID |
| | | | | NO: 120) |
| 68 | 507 | DNA | Artificial | Codon-optimized sequence DnaB_KA_var2 |
| | | | Sequence | encoding Synechocystis sp. PCC6803 DnaB |
| | | | | 4xHisKA-tagged non-ligating mini-intein (SEQ |
| | | | | ID NO: 120) |
| 69 | 507 | DNA | Artificial | Codon-optimized sequence DnaB_KA_var2-1 |
| | | | Sequence | encoding Synechocystis sp. PCC6803 DnaB |
| | | | | 4xHisKA-tagged non-ligating mini-intein (SEQ |
| | | | | ID NO: 120) |
| 70 | 507 | DNA | Artificial | Codon-optimized sequence DnaB_KA_var2-2 |
| | | | Sequence | encoding Synechocystis sp. PCC6803 DnaB |
| | | | | 4xHisKA-tagged non-ligating mini-intein (SEQ |
| | | | | ID NO: 120) |
| 71 | 507 | DNA | Artificial | Codon-optimized sequence DnaB_KA_var2- |
| | | | Sequence | 3_mutant encoding Synechocystis sp. PCC6803 |
| | | | | DnaB 4xHisKA-tagged non-ligating mini-intein |
| | | | | (SEQ ID NO: 120) |
| 72 | 507 | DNA | Artificial | Codon-optimized sequence DnaB_KA_var2-4 |
| | | | Sequence | encoding Synechocystis sp. PCC6803 DnaB |
| | | | | 4xHisKA-tagged non-ligating mini-intein (SEQ |
| | | | | ID NO: 120) |
| 73 | 507 | DNA | Artificial | Codon-optimized sequence DnaB_KA_var2-5 |
| | | | Sequence | encoding Synechocystis sp. PCC6803 DnaB |
| | | | | 4xHisKA-tagged non-ligating mini-intein (SEQ |
| | | | | ID NO: 120) |
| 74 | 507 | DNA | Artificial | Codon-optimized sequence DnaB_KA_var2-6 |
| | | | Sequence | encoding Synechocystis sp. PCC6803 DnaB |
| | | | | 4xHisKA-tagged non-ligating mini-intein (SEQ |
| | | | | ID NO: 120) |
| 75 | 507 | DNA | Artificial | Codon-optimized sequence DnaB_KH encoding |
| | | | Sequence | Synechocystis sp. PCC6803 DnaB 4xHisKH- |
| | | | | tagged non-ligating mini-intein (SEQ ID |
| | | | | NO: 121) |
| 76 | 507 | DNA | Artificial | Codon-optimized sequence DnaB_KS encoding |
| | | | Sequence | Synechocystis sp. PCC6803 DnaB 4xHisKS- |
| | | | | tagged non-ligating mini-intein (SEQ ID NO: 122) |
| 77 | 507 | DNA | Artificial | Codon-optimized sequence DnaB_KT encoding |
| | | | Sequence | Synechocystis sp. PCC6803 DnaB 4xHisKT- |
| | | | | tagged non-ligating mini-intein (SEQ ID NO: 123) |
| 78 | 465 | DNA | Artificial | Codon-optimized sequence DnaB_M86 |
| | | | Sequence | encoding Synechocystis sp. PCC6803 DnaB |
| | | | | non-ligating mini-intein (SEQ ID NO: 124) |
| 79 | 507 | DNA | Artificial | Codon-optimized sequence DnaB_v03 encoding |
| | | | Sequence | Synechocystis sp. PCC6803 DnaB 6xHis- |
| | | | | tagged non-ligating mini-intein (SEQ ID NO: 2) |
| 80 | 507 | DNA | Artificial | Codon-optimized sequence DnaB_v04 encoding |
| | | | Sequence | Synechocystis sp. PCC6803 DnaB 6xHis- |
| | | | | tagged non-ligating mini-intein (SEQ ID NO: 2) |
| 81 | 507 | DNA | Artificial | Codon-optimized sequence DnaB_v05 encoding |
| | | | Sequence | Synechocystis sp. PCC6803 DnaB 6xHis- |
| | | | | tagged non-ligating mini-intein (SEQ ID NO: 2) |
| 82 | 507 | DNA | Artificial | Codon-optimized sequence DnaB_v06 encoding |
| | | | Sequence | Synechocystis sp. PCC6803 DnaB 6xHis- |
| | | | | tagged non-ligating mini-intein (SEQ ID NO: 2) |
| 83 | 507 | DNA | Artificial | Codon-optimized sequence DnaB_v07 encoding |
| | | | Sequence | Synechocystis sp. PCC6803 DnaB 6xHis- |
| | | | | tagged non-ligating mini-intein (SEQ ID NO: 2) |
| 84 | 507 | DNA | Artificial | Codon-optimized sequence DnaB_v08 encoding |
| | | | Sequence | Synechocystis sp. PCC6803 DnaB 6xHis- |
| | | | | tagged non-ligating mini-intein (SEQ ID NO: 2) |
| 85 | 507 | DNA | Artificial | Codon-optimized sequence DnaB_v09 encoding |
| | | | Sequence | Synechocystis sp. PCC6803 DnaB 6xHis- |
| | | | | tagged non-ligating mini-intein (SEQ ID NO: 2) |
| 86 | 507 | DNA | Artificial | Codon-optimized sequence DnaB_v10 encoding |
| | | | Sequence | Synechocystis sp. PCC6803 DnaB 6xHis- |
| | | | | tagged non-ligating mini-intein (SEQ ID NO: 2) |
| 87 | 507 | DNA | Artificial | Codon-optimized sequence DnaB_v11 encoding |
| | | | Sequence | Synechocystis sp. PCC6803 DnaB 6xHis- |
| | | | | tagged non-ligating mini-intein (SEQ ID NO: 2) |
| 88 | 507 | DNA | Artificial | Codon-optimized sequence DnaB_v12 encoding |
| | | | Sequence | Synechocystis sp. PCC6803 DnaB 6xHis- |
| | | | | tagged non-ligating mini-intein (SEQ ID NO: 2) |
| 89 | 507 | DNA | Artificial | Codon-optimized sequence DnaB_v13 encoding |
| | | | Sequence | Synechocystis sp. PCC6803 DnaB 6xHis- |
| | | | | tagged non-ligating mini-intein (SEQ ID NO: 2) |
| 90 | 507 | DNA | Artificial | Codon-optimized sequence DnaB_v14 encoding |
| | | | Sequence | Synechocystis sp. PCC6803 DnaB 6xHis- |
| | | | | tagged non-ligating mini-intein (SEQ ID NO: 2) |
| 91 | 507 | DNA | Artificial | Codon-optimized sequence DnaB_v15 encoding |
| | | | Sequence | Synechocystis sp. PCC6803 DnaB 6xHis- |
| | | | | tagged non-ligating mini-intein (SEQ ID NO: 2) |
| 92 | 507 | DNA | Artificial | Codon-optimized sequence DnaB_v16 encoding |
| | | | Sequence | Synechocystis sp. PCC6803 DnaB 6xHis- |
| | | | | tagged non-ligating mini-intein (SEQ ID NO: 2) |
| 93 | 507 | DNA | Artificial | Codon-optimized sequence DnaB_v17 encoding |
| | | | Sequence | Synechocystis sp. PCC6803 DnaB 6xHis- |
| | | | | tagged non-ligating mini-intein (SEQ ID NO: 2) |
| 94 | 507 | DNA | Artificial | Codon-optimized sequence DnaB_v18 encoding |
| | | | Sequence | Synechocystis sp. PCC6803 DnaB 6xHis- |
| | | | | tagged non-ligating mini-intein (SEQ ID NO: 2) |
| 95 | 507 | DNA | Artificial | Codon-optimized sequence DnaB_v19 encoding |
| | | | Sequence | Synechocystis sp. PCC6803 DnaB 6xHis- |
| | | | | tagged non-ligating mini-intein (SEQ ID NO: 2) |
| 96 | 507 | DNA | Artificial | Codon-optimized sequence DnaB_v20 encoding |
| | | | Sequence | Synechocystis sp. PCC6803 DnaB 6xHis- |
| | | | | tagged non-ligating mini-intein (SEQ ID NO: 2) |
| 97 | 507 | DNA | Artificial | Codon-optimized sequence DnaB_v21 encoding |
| | | | Sequence | Synechocystis sp. PCC6803 DnaB 6xHis- |
| | | | | tagged non-ligating mini-intein (SEQ ID NO: 2) |
| 98 | 507 | DNA | Artificial | Codon-optimized sequence DnaB_v22 encoding |
| | | | Sequence | Synechocystis sp. PCC6803 DnaB 6xHis- |
| | | | | tagged non-ligating mini-intein (SEQ ID NO: 2) |
| 99 | 507 | DNA | Artificial | Codon-optimized sequence DnaB_v23 encoding |
| | | | Sequence | Synechocystis sp. PCC6803 DnaB 6xHis- |
| | | | | tagged non-ligating mini-intein (SEQ ID NO: 2) |
| 100 | 507 | DNA | Artificial | Codon-optimized sequence DnaB_v24 encoding |
| | | | Sequence | Synechocystis sp. PCC6803 DnaB 6xHis- |
| | | | | tagged non-ligating mini-intein (SEQ ID NO: 2) |
| 101 | 507 | DNA | Artificial | Codon-optimized sequence DnaB_v25 encoding |
| | | | Sequence | Synechocystis sp. PCC6803 DnaB 6xHis- |
| | | | | tagged non-ligating mini-intein (SEQ ID NO: 2) |
| 102 | 507 | DNA | Artificial | Codon-optimized sequence DnaB_v26 encoding |
| | | | Sequence | Synechocystis sp. PCC6803 DnaB 6xHis- |
| | | | | tagged non-ligating mini-intein (SEQ ID NO: 2) |
| 103 | 507 | DNA | Artificial | Codon-optimized sequence DnaB_v27 encoding |
| | | | Sequence | Synechocystis sp. PCC6803 DnaB 6xHis- |
| | | | | tagged non-ligating mini-intein (SEQ ID NO: 2) |
| 104 | 507 | DNA | Artificial | Codon-optimized sequence DnaB_v28 encoding |
| | | | Sequence | Synechocystis sp. PCC6803 DnaB 6xHis- |
| | | | | tagged non-ligating mini-intein (SEQ ID NO: 2) |
| 105 | 507 | DNA | Artificial | Codon-optimized sequence DnaB_v29 encoding |
| | | | Sequence | Synechocystis sp. PCC6803 DnaB 6xHis- |
| | | | | tagged non-ligating mini-intein (SEQ ID NO: 2) |
| 106 | 507 | DNA | Artificial | Codon-optimized sequence DnaB_v30 encoding |
| | | | Sequence | Synechocystis sp. PCC6803 DnaB 6xHis- |
| | | | | tagged non-ligating mini-intein (SEQ ID NO: 2) |
| 107 | 507 | DNA | Artificial | Codon-optimized sequence DnaB_v31 encoding |
| | | | Sequence | Synechocystis sp. PCC6803 DnaB 6xHis- |
| | | | | tagged non-ligating mini-intein (SEQ ID NO: 2) |
| 108 | 507 | DNA | Artificial | Codon-optimized sequence DnaB_v32 encoding |
| | | | Sequence | Synechocystis sp. PCC6803 DnaB 6xHis- |
| | | | | tagged non-ligating mini-intein (SEQ ID NO: 2) |
| 109 | 507 | DNA | Artificial | Codon-optimized sequence DnaB_v33 encoding |
| | | | Sequence | Synechocystis sp. PCC6803 DnaB 6xHis- |
| | | | | tagged non-ligating mini-intein (SEQ ID NO: 2) |
| 110 | 507 | DNA | Artificial | Codon-optimized sequence DnaB_v34 encoding |
| | | | Sequence | Synechocystis sp. PCC6803 DnaB 6xHis- |
| | | | | tagged non-ligating mini-intein (SEQ ID NO: 2) |
| 111 | 507 | DNA | Artificial | Codon-optimized sequence DnaB_v35 encoding |
| | | | Sequence | Synechocystis sp. PCC6803 DnaB 6xHis- |
| | | | | tagged non-ligating mini-intein (SEQ ID NO: 2) |
| 112 | 507 | DNA | Artificial | Codon-optimized sequence DnaB_v36 encoding |
| | | | Sequence | Synechocystis sp. PCC6803 DnaB 6xHis- |
| | | | | tagged non-ligating mini-intein (SEQ ID NO: 2) |
| 113 | 507 | DNA | Artificial | Codon-optimized sequence DnaB_v37 encoding |
| | | | Sequence | Synechocystis sp. PCC6803 DnaB 6xHis- |
| | | | | tagged non-ligating mini-intein (SEQ ID NO: 2) |
| 114 | 507 | DNA | Artificial | Codon-optimized sequence DnaB_v38 encoding |
| | | | Sequence | Synechocystis sp. PCC6803 DnaB 6xHis- |
| | | | | tagged non-ligating mini-intein (SEQ ID NO: 2) |
| 115 | 507 | DNA | Artificial | Codon-optimized sequence DnaB_v39 encoding |
| | | | Sequence | Synechocystis sp. PCC6803 DnaB 6xHis- |
| | | | | tagged non-ligating mini-intein (SEQ ID NO: 2) |
| 116 | 507 | DNA | Artificial | Codon-optimized sequence DnaB_v40 encoding |
| | | | Sequence | Synechocystis sp. PCC6803 DnaB 6xHis- |
| | | | | tagged non-ligating mini-intein (SEQ ID NO: 2) |
| 117 | 507 | DNA | Artificial | Codon-optimized sequence DnaB_v41 encoding |
| | | | Sequence | Synechocystis sp. PCC6803 DnaB 6xHis- |
| | | | | tagged non-ligating mini-intein (SEQ ID NO: 2) |
| 118 | 507 | DNA | Artificial | Codon-optimized sequence DnaB_v42 encoding |
| | | | Sequence | Synechocystis sp. PCC6803 DnaB 6xHis- |
| | | | | tagged non-ligating mini-intein (SEQ ID NO: 2) |
| 119 | 507 | DNA | Artificial | Codon-optimized sequence DnaB_v43 encoding |
| | | | Sequence | Synechocystis sp. PCC6803 DnaB 6xHis- |
| | | | | tagged non-ligating mini-intein (SEQ ID NO: 2) |
| 120 | 169 | PRT | Artificial | Codon-optimized sequence DnaB_KA_var2-6 |
| | | | Sequence | encoding Synechocystis sp. PCC6803 DnaB |
| | | | | 4xHisKA-tagged non-ligating mini-intein |
| 121 | 169 | PRT | Artificial | Codon-optimized sequence DnaB_KA_var2-6 |
| | | | Sequence | encoding Synechocystis sp. PCC6803 DnaB |
| | | | | 4xHisKH-tagged non-ligating mini-intein |
| 122 | 169 | PRT | Artificial | Codon-optimized sequence DnaB_KA_var2-6 |
| | | | Sequence | encoding Synechocystis sp. PCC6803 DnaB |
| | | | | 4xHisKS-tagged non-ligating mini-intein |
| 123 | 169 | PRT | Artificial | Codon-optimized sequence DnaB_KA_var2-6 |
| | | | Sequence | encoding Synechocystis sp. PCC6803 DnaB |
| | | | | 4xHisKT-tagged non-ligating mini-intein |
| 124 | 155 | PRT | Artificial | Synechocystis sp. PCC6803, DnaB; 6xHis- |
| | | | Sequence | tagged mini-intein variant M86 |

We claim:

1. An intein polypeptide comprising an amino acid sequence having at least 70% amino acid sequence identity to an amino acid sequence selected from the group consisting of amino acids 105-124 of SEQ ID NO:2, amino acids 93-112 of SEQ ID NO:1, amino acids 85-104 of SEQ ID NO:4, amino acids 1-21 of SEQ ID NO:12, amino acids 1-21 of SEQ ID NO:15, amino acids 105-124 of SEQ ID NO:120, amino acids 105-124 of SEQ ID NO:121, amino acids 105-124 of SEQ ID NO:122, amino acids 105-124 of SEQ ID NO:123, and amino acids 105-124 of SEQ ID NO:124; or an intein polypeptide comprising an amino acid sequence having at least 60% amino acid sequence identity to an amino acid sequence selected from the group consisting of amino acids 100-129 of SEQ ID NO:2, amino acids 88-117 of SEQ ID NO:1, amino acids 80-109 of SEQ ID NO:4, amino acids 1-30 of SEQ ID NO:12, amino acids 1-30 of SEQ ID NO:15, amino acids 100-129 of SEQ ID NO:120, amino acids 100-129 of SEQ ID NO:121, amino acids 100-129 of SEQ ID NO:122, amino acids 100-129 of SEQ ID NO:123, and amino acids 100-129 of SEQ ID NO:124.

2. The intein polypeptide of claim 1, comprising an amino acid sequence having at least 80% amino acid sequence identity to an amino acid sequence selected from the group consisting of amino acids 105-124 of SEQ ID NO:2, amino acids 93-112 of SEQ ID NO:1, amino acids 85-104 of SEQ ID NO:4, amino acids 1-21 of SEQ ID NO:12, amino acids 1-21 of SEQ ID NO:15, amino acids 105-124 of SEQ ID NO:120, amino acids 105-124 of SEQ ID NO:121, amino acids 105-124 of SEQ ID NO:122, amino acids 105-124 of SEQ ID NO:123, and amino acids 105-124 of SEQ ID NO:124; or comprising an amino acid sequence having at least 70% amino acid sequence identity to an amino acid sequence selected from the group consisting of amino acids 100-129 of SEQ ID NO:2, amino acids 88-117 of SEQ ID NO:1, amino acids 80-109 of SEQ ID NO:4, amino acids 1-30 of SEQ ID NO:12, amino acids 1-30 of SEQ ID NO:15, amino acids 100-129 of SEQ ID NO:120, amino acids 100-129 of SEQ ID NO:121, amino acids 100-129 of SEQ ID NO:122, amino acids 100-129 of SEQ ID NO:123, and amino acids 100-129 of SEQ ID NO:124.

3. The intein polypeptide of claim 2, comprising an amino acid sequence having at least 90% amino acid sequence identity to an amino acid sequence selected from the group consisting of amino acids 105-124 of SEQ ID NO:2, amino acids 93-112 of SEQ ID NO:1, amino acids 85-104 of SEQ ID NO:4, amino acids 1-21 of SEQ ID NO:12, amino acids 1-21 of SEQ ID NO:15, amino acids 105-124 of SEQ ID NO:120, amino acids 105-124 of SEQ ID NO:121, amino acids 105-124 of SEQ ID NO:122, amino acids 105-124 of SEQ ID NO:123, and amino acids 105-124 of SEQ ID NO:124; or comprising an amino acid sequence having at least 80% amino acid sequence identity to an amino acid sequence selected from the group consisting of amino acids 88-117 of SEQ ID NO:1, amino acids 100-129 of SEQ ID NO:2, amino acids 80-109 of SEQ ID NO:4, amino acids 1-30 of SEQ ID NO:12, amino acids 1-30 of SEQ ID NO:15, amino acids 100-129 of SEQ ID NO:120, amino acids 100-129 of SEQ ID NO:121, amino acids 100-129 of SEQ ID NO:122, amino acids 100-129 of SEQ ID NO:123, and amino acids 100-129 of SEQ ID NO:124.

4. The intein polypeptide of claim 3, comprising an amino acid sequence having at least 95% amino acid sequence identity to an amino acid sequence selected from the group consisting of amino acids 105-124 of SEQ ID NO:2, amino acids 93-112 of SEQ ID NO:1, amino acids 85-104 of SEQ ID NO:4, amino acids 1-21 of SEQ ID NO:12, amino acids 1-21 of SEQ ID NO:15, amino acids 105-124 of SEQ ID NO:120, amino acids 105-124 of SEQ ID NO:121, amino acids 105-124 of SEQ ID NO:122, amino acids 105-124 of SEQ ID NO:123, and amino acids 105-124 of SEQ ID NO:124; or comprising an amino acid sequence having at least 90% amino acid sequence identity to an amino acid sequence selected from the group consisting of amino acids 88-117 of SEQ ID NO:1, amino acids 100-129 of SEQ ID NO:2, amino acids 80-109 of SEQ ID NO:4, amino acids 1-30 of SEQ ID NO:12, amino acids 1-30 of SEQ ID NO:15, amino acids 100-129 of SEQ ID NO:120, amino acids 100-129 of SEQ ID NO:121, amino acids 100-129 of SEQ ID NO:122, amino acids 100-129 of SEQ ID NO:123, and amino acids 100-129 of SEQ ID NO:124.

5. The intein polypeptide of claim 4, comprising an amino acid sequence selected from the group consisting of amino acids 105-124 of SEQ ID NO:2, amino acids 93-112 of SEQ ID NO:1, amino acids 85-104 of SEQ ID NO:4, amino acids 1-21 of SEQ ID NO:12, amino acids 1-21 of SEQ ID NO:15, amino acids 105-124 of SEQ ID NO:120, amino acids 105-124 of SEQ ID NO:121, amino acids 105-124 of SEQ ID NO:122, amino acids 105-124 of SEQ ID NO:123, and amino acids 105-124 of SEQ ID NO:124; or comprising an amino acid sequence selected from the group consisting of amino acids 88-117 of SEQ ID NO:1, amino acids 100-129 of SEQ ID NO:2, amino acids 80-109 of SEQ ID NO:4, amino acids 1-30 of SEQ ID NO:12, amino acids 1-30 of SEQ ID NO:15, amino acids 100-129 of SEQ ID NO:120, amino acids 100-129 of SEQ ID NO:121, amino acids 100-129 of SEQ ID NO:122, amino acids 100-129 of SEQ ID NO:123, and amino acids 100-129 of SEQ ID NO:124.

6. The intein polypeptide of any one of claims 1 to 5, wherein the intein amino acid sequence lacks an N-terminal cysteine residue.

7. The intein polypeptide of any one of claims 1 to 6, wherein the intein polypeptide lacks substantial extein-ligating activity.

8. The intein polypeptide of any one of claims 1 to 5, wherein the intein polypeptide comprises a polyhistidine tag.

9. The intein polypeptide of claim 8, wherein the polyhistidine tag comprises a 6×His tag.

10. The intein polypeptide of any one of claims 1 to 5, wherein the intein polypeptide comprises a cleavage sequence cleavable by a protease.

11. The intein polypeptide of any one of claims 1 to 5, comprising the amino acid sequence of any one of SEQ ID NOs: 1, 2, 4, 12, 15, and 120-124

12. An intein fusion polypeptide comprising the amino acid sequence of an intein polypeptide of any one of claims 1 to 5, and an amino acid sequence of a target polypeptide.

13. The intein fusion polypeptide of claim 12, wherein the N-terminal amino acid of the amino acid sequence of the target polypeptide is the N-terminal amino acid of the amino acid sequence of a mature form of the target protein.

14. The intein fusion polypeptide of claim 13, wherein the target polypeptide can form one or more disulfide bonds.

15. The intein fusion polypeptide of claim 12, wherein the target polypeptide comprises a polypeptide selected from the group consisting of: an antibody heavy chain, an antibody light chain, and fragments thereof.

16. The intein fusion polypeptide of claim 12, wherein the intein fusion polypeptide lacks a signal sequence.

17. The intein fusion polypeptide of claim 12, wherein the intein amino acid sequence lacks an N-terminal cysteine residue.

18. The intein fusion polypeptide of claim 12, wherein the intein fusion polypeptide lacks substantial extein-ligating activity.

19. The intein fusion polypeptide of claim 12, wherein the intein fusion polypeptide comprises a polyhistidine tag.

20. The intein fusion polypeptide of claim 19, wherein the polyhistidine tag comprises a 6×His tag.

21. The intein fusion polypeptide of claim 12, wherein the intein fusion polypeptide comprises a cleavage sequence cleavable by a protease.

22. A polynucleotide encoding the intein polypeptide of any one of claims 1 to 5.

23. A polynucleotide encoding an intein polypeptide and comprising a nucleotide sequence selected from the group consisting of SEQ ID NOs 24-119.

24. An expression construct comprising two or more intein-encoding polynucleotide sequences, wherein each intein-encoding polynucleotide sequence differs from every other intein-encoding polynucleotide sequence, and wherein every intein polypeptide encoded by the intein-encoding polynucleotide sequences has the same amino acid sequence, or wherein at least two of the intein polypeptides encoded by the intein-encoding polynucleotide sequences have different amino acid sequences.

25. The expression construct of claim 24, wherein the two or more intein encoding polynucleotide sequences are selected from the group consisting of SEQ ID NOs: 24-119.

26. The expression construct of claim 24, wherein the intein-encoding polynucleotide sequences, and the amino acid sequence of the intein polypeptide encoded by those intein-encoding polynucleotide sequences, are selected from the group consisting of:
(a) polynucleotide sequences SEQ ID NOs 26 and 27, and amino acid sequence SEQ ID NO:2;
(b) polynucleotide sequences SEQ ID NOs 24 and 25, and amino acid sequence SEQ ID NO:1;
(c) polynucleotide sequences SEQ ID NOs 28 and 29, and amino acid sequence SEQ ID NO:3;
(d) polynucleotide sequences SEQ ID NOs 30 and 31, and amino acid sequence SEQ ID NO:4;
(e) polynucleotide sequences SEQ ID NOs 32 and 33, and amino acid sequence SEQ ID NO:5;
(f) polynucleotide sequences SEQ ID NOs 34 and 35, and amino acid sequence SEQ ID NO:6;
(g) polynucleotide sequences SEQ ID NOs 36 and 37, and amino acid sequence SEQ ID NO:7;
(h) polynucleotide sequences SEQ ID NOs 38 and 39, and amino acid sequence SEQ ID NO:8;
(i) polynucleotide sequences SEQ ID NOs 40 and 41, and amino acid sequence SEQ ID NO:9;
(j) polynucleotide sequences SEQ ID NOs 43 and 44, and amino acid sequence SEQ ID NO:11;
(k) polynucleotide sequences SEQ ID NOs 45 and 46, and amino acid sequence SEQ ID NO:12;
(l) polynucleotide sequences SEQ ID NOs 48 and 49, and amino acid sequence SEQ ID NO:14;
(m) polynucleotide sequences SEQ ID NOs 50 and 51, and amino acid sequence SEQ ID NO:15;
(n) polynucleotide sequences SEQ ID NOs 53 and 54, and amino acid sequence SEQ ID NO:17;
(o) polynucleotide sequences SEQ ID NOs 56 and 57, and amino acid sequence SEQ ID NO:19;
(p) polynucleotide sequences SEQ ID NOs 59 and 60, and amino acid sequence SEQ ID NO:21; and
(q) any two or more of polynucleotide sequences SEQ ID NOs 26, 27, 61-66, and 79-119, and amino acid sequence SEQ ID NO:2.

27. The expression construct of any one of claims 24 to 26, wherein the expression construct is an expression vector.

28. The expression construct of claim 27, wherein the expression construct is a dual-promoter expression vector.

29. The expression construct of claim 28, wherein the dual-promoter expression vector comprises an L-arabinose-inducible promoter and a propionate-inducible promoter.

30. A method for producing a target polypeptide, the method comprising:
generating a composition comprising the intein fusion polypeptide of claim 12, wherein the intein amino acid sequence self-excises from the intein fusion polypeptide, thereby producing the target polypeptide; and
recovering the target polypeptide from the composition.

31. The method of claim 30, wherein generating the composition comprises expressing the intein fusion protein in a host cell.

32. The method of claim 31, wherein generating the composition further comprises lysing the host cell.

33. The method of claim 32, wherein the composition is the lysate generated by lysing the host cell.

34. The method of any one of claims 30 to 33, wherein the host cell has a reduced level of function of thioredoxin reductase and a reduced level of function of a protein selected from the group consisting of glutathione reductase and glutathione synthetase.

35. The method of claim 34, wherein the host cell has an altered form of the gene encoding AhpC selected from the group consisting of the ahpC*, ahpC^{Δ}, V164G, S71F, E173/S71F, E171Ter, and dup162-169 mutations.

36. The method of claim 34 or claim 35, wherein the host cell comprises a polynucleotide encoding a cytoplasmic form of DsbC.

37. The method of any one of claims 31 to 33, wherein the host cell is a prokaryotic cell.

38. The method of claim 37, wherein the host cell is Escherichia coli.

39. The method of claim 38, wherein the host cell is an Escherichia coli B strain 521 cell.

40. A host cell comprising the expression construct of any one of claims 24 to 29, wherein the host cell has a reduced level of function of thioredoxin reductase and a reduced level of function of a protein selected from the group consisting of glutathione reductase and glutathione synthetase.

41. The host cell of claim 40, wherein the host cell has an altered form of the gene encoding AhpC selected from the group consisting of the ahpC*, ahpC^{Δ}, V164G, S71F, E173/S71F, E171Ter, and dup162-169 mutations.

42. The host cell of claim 40 or claim 41, wherein the host cell comprises a polynucleotide encoding a cytoplasmic form of DsbC.

43. The host cell of claim 40, wherein the host cell is a prokaryotic cell.

44. The host cell of claim 43, wherein the host cell is Escherichia coli.

45. The host cell of claim 44, wherein the host cell is an Escherichia coli B strain 521 cell.


SEQUENCE LISTING (124 sequences)

SEQ ID NO: 1 - protein, 151 aa, Artificial Sequence
  Synechocystis sp. PCC6803 DnaX 6xHis-tagged non-ligating mini-intein
MALTGDSQVLTRNGLMSIDNPQIKGREVLSYNETLQQWEYKKVLRWLDRGEKQTLSIKTKNSTVRCTANHLIRTEQGWTRAENITPGMKILSPASASGHHHHHHGGSGSSPQWHTNFEEVESVTKGQVEKVYDLEVEDNHNFVANGLLVHN

SEQ ID NO: 2 - protein, 169 aa, Artificial Sequence
  Synechocystis sp. PCC6803 DnaB 6xHis-tagged non-ligating mini-intein
MAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLASGHHHHHHGGSGSSPEIEKLSQSDIYWDSIVSITETGVEEVFDLTVPGPHNFVANDIIVHN

SEQ ID NO: 3 - protein, 140 aa, Artificial Sequence
  Desulfofundulus thermosubterraneus DSM 16057 DnaB non-ligating intein
MALAGETLVQLASGHRVPIKDLVREKQPVKVMALNEITWKLEPAAVSKVWCTGVKPVFRLCTQLGREVRATANHKFRTLEGWKCLGELQPGEFIALPRSLESDVFWDRVVSIEPDGIEEVYDLEVPGHHNFVAADIIVHN

SEQ ID NO: 4 - protein, 136 aa, Artificial Sequence
  Prochlorococcus cyanophage P-SSM2 gp41-1 6xHis-tagged non-ligating mini-intein
MALDLKTQVQTPQGMKEISNIQVGDLVLSNTGYNEVLNVFPKSKKKSYKITLEDGKEIICSEEHLFPTQTGEMNISGGLKEGMCLYVKEGHHHHHHGGSGMLKKILKIEELDERELIDIEVSGNHLFYANDILTHN

SEQ ID NO: 5 - protein, 140 aa, Artificial Sequence
  Nostoc punctiforme strain ATCC 29133 / PCC 73102 DnaB non-ligating mini-intein
MALAGDSLVTLVDSGLQVPIKELVGKSGFAVWALNEATMQLEKAIVSNAFSTGIKPLFTLTTRLGRKIRATGNHKFLTINGWKRLDELTPKEHLALPRNSGSDIYWDEIVSITYSGEEEVFDLTVPGLHNFVANNIIVHN

SEQ ID NO: 6 - protein, 170 aa, Artificial Sequence
  Mycobacterium tuberculosis strain ATCC 25618 / H37Rv RecA non-ligating mini-intein
MALAEGTRIFDPVTGTTHRIEDVVGGRKPIHVVAAAKDGTLHARPVVSWFDQGTRDVIGLRIAGGAILWATPDHKVLTEYGWRAAGELRKGDRVAQPRRFDGFGDSAPIPADRVQALADALDDKFLHDMLAEELRYSVIREVLPTRRARTFGLEVEELHTLVAEGVVVHN

SEQ ID NO: 7 - protein, 136 aa, Artificial Sequence
  Clostridium thermocellum CthBIL4 N115D non-ligating intein
MAFVAGTMILTATGLVAIENIKAGDKVIATNPETFEVAEKTVLETYVRETTELLHLTIGGEVIKTTFDHPFYVKDVGFVEAGKLQVGDKLLDSRGNVLVVEEKKLEIADKPVKVYDFKVDDFHTYHVGDNEVLVHN

SEQ ID NO: 8 - protein, 168 aa, Artificial Sequence
  Deinococcus swuensis PolIII subunit alpha non-ligating mini-intein
MALTGDTRVPVAGGELRRIEDLCGEGQPVQLPSVNGEYRLELRPTGQFFDNGVKPVFKVKTALGRELTATGNHPLLTLDGWWRDVEAQSGVCTKEFYGTPKAHKKGFRRSTIQTLGEFFGATELLDACLDDLYWDTITSITPAGEAQTFDLEVPGTHNFVANDLIVHN

SEQ ID NO: 9 - protein, 163 aa, Artificial Sequence
  Cyanobacterium aponinum DnaB non-ligating intein
MALSGDSLIALADTNTVVPIRELVSKKDITVFALNEATMKIEKAKVINAFCTGIKPVFCLKTRLGRTIKATANHKFFTIEGWKRLDELSVKDFIALPRYLPSSEISTIKLGEIVQCDVIKSLANSDIYWDEITSIEPLGETEVYDLTVENYHNFIANNIIVHN

SEQ ID NO: 10 - protein, 95 aa, Artificial Sequence
  Synechocystis sp. PCC6803 DnaX non-ligating split intein, N-terminal portion
MALTGDSQVLTRNGLMSIDNPQIKGREVLSYNETLQQWEYKKVLRWLDRGEKQTLSIKTKNSTVRCTANHLIRTEQGWTRAENITPGMKILSPAS

SEQ ID NO: 11 - protein, 43 aa, Artificial Sequence
  Synechocystis sp. PCC6803 DnaX non-ligating split intein, C-terminal portion
MSPQWHTNFEEVESVTKGQVEKVYDLEVEDNHNFVANGLLVHN

SEQ ID NO: 12 - protein, 61 aa, Artificial Sequence
  Synechocystis sp. PCC6803 DnaX 6xHis-tagged non-ligating split intein, C-terminal portion
MHHHHHHDYKDDDDKGSGSSPQWHTNFEEVESVTKGQVEKVYDLEVEDNHNFVANGLLVHN

SEQ ID NO: 13 - protein, 107 aa, Artificial Sequence
  Synechocystis sp. PCC6803 DnaB non-ligating split intein, N-terminal portion
MAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQL

SEQ ID NO: 14 - protein, 49 aa, Artificial Sequence
  Synechocystis sp. PCC6803 DnaB non-ligating split intein, C-terminal portion
MSPEIEKLSQSDIYWDSIVSITETGVEEVFDLTVPGPHNFVANDIIVHN

SEQ ID NO: 15 - protein, 67 aa, Artificial Sequence
  Synechocystis sp. PCC6803 DnaB 6xHis-tagged non-ligating split intein, C-terminal portion
MHHHHHHDYKDDDDKGSGSSPEIEKLSQSDIYWDSIVSITETGVEEVFDLTVPGPHNFVANDIIVHN

SEQ ID NO: 16 - protein, 89 aa, Artificial Sequence
  Prochlorococcus cyanophage P-SSM2 gp41-1 non-ligating split intein, N-terminal portion
MALDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 17 - protein, 36 aa, Artificial Sequence
  Prochlorococcus cyanophage P-SSM2 gp41-1 non-ligating split intein, C-terminal portion
MLKKILKIEELDERELIDIEVSGNHLFYANDILTHN

SEQ ID NO: 18 - protein, 94 aa, Artificial Sequence
  Clostridium thermocellum CthBIL4 N115D non-ligating split intein ′C42′, N-terminal portion
MAFVAGTMILTATGLVAIENIKAGDKVIATNPETFEVAEKTVLETYVRETTELLHLTIGGEVIKTTFDHPFYVKDVGFVEAGKLQVGDKLLDSR

SEQ ID NO: 19 - protein, 43 aa, Artificial Sequence
  Clostridium thermocellum CthBIL4 N115D non-ligating split intein ′C42′, C-terminal portion
MGNVLVVEEKKLEIADKPVKVYDFKVDDFHTYHVGDNEVLVHN

SEQ ID NO: 20 - protein, 120 aa, Artificial Sequence
  Clostridium thermocellum CthBIL4 N115D non-ligating split intein ′C16′, N-terminal portion
MAFVAGTMILTATGLVAIENIKAGDKVIATNPETFEVAEKTVLETYVRETTELLHLTIGGEVIKTTFDHPFYVKDVGFVEAGKLQVGDKLLDSRGNVLVVEEKKLEIADKPVKVYDFKVD

SEQ ID NO: 21 - protein, 17 aa, Artificial Sequence
  Clostridium thermocellum CthBIL4 N115D non-ligating split intein ′C16′, C-terminal portion
MDFHTYHVGDNEVLVHN

SEQ ID NO: 22 - protein, 227 aa, Artificial Sequence
  TRAST-Fab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 23 - protein, 214 aa, Artificial Sequence
  TRAST-Fab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 453 bp, Artificial Sequence
  Codon-optimized sequence #1 encoding Synechocystis sp. PCC6803 DnaX 6xHis-tagged non-ligating mini-intein (SEQ ID NO1)
atggctcttaccggagactcgcaggtccttacacgcaacgggttaatgtctattgacaacccacagattaagggtcgtgaggtactttcgtacaatgaaacacttcaacagtgggaatataagaaagtcttacgttggttggatcgcggcgagaagcaaactctttcaatcaaaacaaaaaactcgacggttcgctgtacagccaaccacttaattcgtaccgaacaggggtggacacgtgcggaaaatatcacgccgggcatgaaaattctttcccctgccagcgcaagtggtcatcatcaccatcatcatgggggtagcggatcatcgccacaatggcataccaattttgaggaggtggagtcggttacaaaaggtcaggtggaaaaggtatatgaccttgaggtagaagacaatcacaattttgttgcaaatgggcttttagttcataac

SEQ ID NO: 25 - DNA, 453 bp, Artificial Sequence
  Codon-optimized sequence #2 encoding Synechocystis sp. PCC6803 DnaX 6xHis-tagged non-ligating mini-intein (SEQ ID NO1)
atggccttaacaggggactcccaagtgttgacccgtaatggtttaatgagtatcgataacccgcagatcaagggtcgtgaagtattatcatataatgaaacgctgcaacagtgggaatacaaaaaggttttacgttggctggaccgtggggagaagcagacactgtcgatcaagacgaaaaactccaccgtccgctgcactgcaaaccaccttatccgcaccgaacaaggctggacccgcgcagagaacatcacacctggaatgaaaatccttagtcctgcgtctgcctcaggacaccatcaccatcatcatgggggaagtgggtccagtccgcagtggcatactaatttcgaggaggtagagtcagtcactaagggtcaggtggagaaggtttatgatttggaagtggaagacaatcataatttcgtggcaaatggtctgcttgttcataac

SEQ ID NO: 26 - DNA, 507 bp, Artificial Sequence
  Codon-optimized sequence #1 encoding Synechocystis sp. PCC6803 DnaB 6xHis-tagged non-ligating mini-intein (SEQ ID NO2)
atggctatttcaggcgacagtctgatctctcttgcttccacagggaagcgcgtatctattaaagatttgttggacgaaaaagattttgaaatttgggctatcaacgagcaaaccatgaagctggaatctgcaaaggtcagccgcgtcttctgcacaggtaaaaaacttgtttacattcttaaaacgcgtctgggtcgtacaatcaaggcgacagccaaccatcgctttttaaccattgatggttggaagcgtctggatgagttgtctctgaaggagcacatcgcgcttcctcgtaaactggagtcgtctagtttgcaattggcctccgggcaccatcaccatcatcatggggggtcggggtcttcgccggagatcgaaaagttgtcccagtccgacatctactgggattcgatcgttagcatcactgagacaggagtggaagaggtgttcgacttaacggtgcctggaccccataactttgtggcgaacgatatcatcgttcacaac

SEQ ID NO: 27 - DNA, 507 bp, Artificial Sequence
  Codon-optimized sequence #2 encoding Synechocystis sp. PCC6803 DnaB 6xHis-tagged non-ligating mini-intein (SEQ ID NO2)
atggccattagcggagactccctgatctctttggcttctaccggaaagcgcgtaagcatcaaagacctgcttgatgagaaagatttcgaaatctgggcgattaacgagcaaactatgaaattggagtctgctaaggtaagtcgcgtattctgcactgggaaaaagttagtttatattttgaaaacacgtttagggcgcactattaaggcaacagcaaatcaccgctttctgaccattgatggttggaaacgtctggatgagcttagtcttaaagaacacatcgctttgcctcgcaaacttgaatcgtcatcgcttcaactggcgtcaggccatcatcatcaccatcatgggggatcgggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 28 - DNA, 420 bp, Artificial Sequence
  Codon-optimized sequence #1 encoding Desulfofundulus thermosubterraneus DSM 16057 DnaB non-ligating intein (SEQ ID NO3)
atggcattagccggtgaaaccctggtacaactggcctccggtcaccgtgtcccaatcaaagaccttgtacgtgagaaacagcccgttaaggttatggcactgaatgaaatcacatggaaacttgaacctgctgccgtctctaaggtttggtgcactggtgtaaaacccgtattccgcttatgcacgcaacttggccgtgaggtacgcgcgacagctaatcataaattccgcaccttagaaggttggaagtgtcttggagagttgcaaccgggagagtttatcgccttgcctcgtagtctggagagcgatgtgttctgggaccgcgtcgtctcaatcgaaccggatgggatcgaggaagtctatgatctggaagtccctggacatcacaattttgtagccgccgatattatcgtccataat

SEQ ID NO: 29 - DNA, 420 bp, Artificial Sequence
  Codon-optimized sequence #2 encoding Desulfofundulus thermosubterraneus DSM 16057 DnaB non-ligating intein (SEQ ID NO3)
atggcgctggcaggcgaaacattagtccaacttgcatctggtcaccgcgtgccaatcaaggatcttgtgcgcgagaaacaaccagttaaggtcatggcacttaacgagatcacttggaaactggaacctgcggctgtctccaaagtttggtgtacgggggttaagccggtgttccgcttgtgcacgcaactggggcgtgaagtacgtgcgacggcaaatcataagtttcgcacattagaaggttggaaatgccttggtgaattacaacctggagagtttattgccttgccgcgctcactggagagcgatgttttttgggaccgtgttgtctccattgaaccggatggtattgaggaagtttacgaccttgaggttccaggccaccacaactttgtagcagccgacattattgtccataat

SEQ ID NO: 30 - DNA, 408 bp, Artificial Sequence
  Codon-optimized sequence #1 encoding Prochlorococcus cyanophage P-SSM2 gp41-1 6xHis-tagged non-ligating mini-intein (SEQ ID NO4)
atggccctggatctgaagacgcaagttcagacgccgcagggtatgaaggaaatctcgaacattcaagtcggtgatttagtgcttagcaatacgggctacaacgaagtcctgaacgtatttcctaaatcaaaaaagaaatcctacaagattactttagaagacgggaaggagatcatttgtagtgaagagcacttgtttcctacccaaaccggagagatgaatatcagcggtggtcttaaggaaggtatgtgtttatacgtgaaagagggacaccatcatcatcaccacggtgggagtggtatgcttaagaaaatcttgaagattgaggagttagatgagcgcgaactgattgacattgaggtgtcaggaaaccatctgttctatgccaacgacattcttacgcacaac

SEQ ID NO: 31 - DNA, 408 bp, Artificial Sequence
  Codon-optimized sequence #2 encoding Prochlorococcus cyanophage P-SSM2 gp41-1 6xHis-tagged non-ligating mini-intein (SEQ ID NO4)
atggccctggacttaaagacacaagtgcagaccccacaaggtatgaaagagatcagtaacatccaagtcggagacttagtgctgagtaatacgggatacaatgaggtgctgaatgttttcccgaagtctaagaagaaaagttataaaattacgctggaggatggcaaggagattatttgctcagaggaacatcttttccccacacaaacgggcgagatgaacatttcgggcggccttaaagagggaatgtgcttatatgtcaaagagggacatcaccatcatcatcatggaggttctggcatgttgaaaaagattttgaagatcgaagagttggacgaacgcgaattgatcgacatcgaggtatcaggcaaccatttattttatgcgaatgacatcttaacacacaac

SEQ ID NO: 32 - DNA, 420 bp, Artificial Sequence
  Codon-optimized sequence #1 encoding Nostoc punctiforme strain ATCC 29133 / PCC 73102 DnaB non-ligating mini-intein (SEQ ID NO5)
atggccctggccggagacagtttggtaacattagttgattcggggctgcaagtcccaatcaaggaactggtcggcaagtcaggatttgcagtatgggccttaaacgaagcgaccatgcagttggagaaagcgattgtaagtaatgctttctcaacaggtatcaagccattattcacccttaccacccgcttaggccgcaagatccgtgcaacaggaaatcataaatttctgaccattaacggttggaagcgtctggacgagttgacacccaaagagcatcttgcgttgcctcgcaactctgggagcgacatttactgggatgagatcgttagtattacctactcaggtgaagaggaagtgttcgatttgaccgttccgggtcttcataattttgtagctaataatattatcgttcataac

SEQ ID NO: 33 - DNA, 420 bp, Artificial Sequence
  Codon-optimized sequence #2 encoding Nostoc punctiforme strain ATCC 29133 / PCC 73102 DnaB non-ligating mini-intein (SEQ ID NO5)
atggctttagcgggagactctctggttactctggttgacagcgggttacaggttccaattaaagaacttgttggaaagtcgggattcgctgtttgggcactgaacgaggctactatgcagcttgagaaagcaatcgtctcaaacgcgttctctacgggtattaagcccctttttactcttacgacccgcttgggccgcaagatccgtgcgactggtaaccataaattccttaccatcaacggctggaagcgcctggatgagctgactcctaaggagcatttagctcttccacgcaattcaggatctgatatttactgggatgaaattgtgtctatcacgtattctggcgaagaggaggtattcgacctgactgtgccgggtttgcataacttcgtcgcaaacaacattattgtccataac

SEQ ID NO: 34 - DNA, 510 bp, Artificial Sequence
  Codon-optimized sequence #1 encoding Mycobacterium tuberculosis strain ATCC 25618 / H37Rv RecA non-ligating mini-intein (SEQ ID NO6)
atggccttggctgaagggacacgtatcttcgatccagttacgggtacaacgcaccgcatcgaagatgtagtgggtggtcgtaagccgattcatgtagttgctgctgcgaaagacgggactttgcacgcgcgtcctgtagtttcctggttcgatcagggaactcgtgacgttattggtcttcgcattgcagggggtgccattttatgggccacgccagatcacaaagttcttaccgagtacgggtggcgtgcagcaggcgaacttcgtaaaggggatcgcgtggcacagcctcgccgctttgatgggtttggtgattctgctcccattccagcagatcgtgtacaagcgttggctgatgccctggacgacaaattcttacatgatatgcttgctgaggagttgcgctattcagttattcgcgaagttttacctactcgccgcgcccgcacgttcggtctggaagtggaggaactgcacactttagtagctgaaggagtagtagtgcacaat

SEQ ID NO: 35 - DNA, 510 bp, Artificial Sequence
  Codon-optimized sequence #2 encoding Mycobacterium tuberculosis strain ATCC 25618 / H37Rv RecA non-ligating mini-intein (SEQ ID NO6)
atggcccttgcagaaggtacacgcatttttgaccccgtcactggcaccacccaccgcatcgaggacgtggtaggcgggcgcaaaccgatccatgtagtagctgcggctaaagatggaactttgcacgctcgtccggtagtctcgtggtttgatcaggggacacgcgacgttatcggattacgtatcgcgggtggggctattctttgggccacaccagaccacaaagtacttaccgagtatggttggcgtgcagccggcgaactgcgtaaaggtgatcgtgttgcacaaccccgtcgctttgacggattcggggactctgcgccgatccctgctgatcgcgtgcaggcacttgccgatgctcttgatgataaattcttgcacgacatgttagcggaagaacttcgttacagtgtaattcgtgaggtattaccgacccgccgcgctcgcacatttggattagaagtggaggagctgcatacactggtcgctgaaggtgtcgttgttcataat

SEQ ID NO: 36 - DNA, 408 bp, Artificial Sequence
  Codon-optimized sequence #1 encoding Clostridium thermocellum CthBIL4 N115D engineered BIL (bacterial non-ligating intein-like) (SEQ ID NO7)
atggcttttgtagcgggcaccatgattcttacagctaccggcttagtcgccatcgaaaatatcaaggcaggcgacaaggtaatcgcgacaaatcctgagacttttgaggtcgccgagaagactgtattggaaacctatgtccgtgagacaactgaattgctgcacctgacgattggtggtgaagtgattaaaaccactttcgatcaccccttctacgtaaaggatgtaggttttgtggaggccggtaaattacaggttggggacaagctgttagattcgcgcggtaacgttctggtggtcgaagaaaagaaacttgagattgcagacaaacccgtgaaagtctacgacttcaaggtggacgactttcacacttaccacgtcggagataatgaagtgcttgtccataac

SEQ ID NO: 37 - DNA, 408 bp, Artificial Sequence
  Codon-optimized sequence #2 encoding Clostridium thermocellum CthBIL4 N115D engineered BIL (bacterial non-ligating intein-like) (SEQ ID NO7)
atggcgtttgtagcaggcacgatgattcttacggctaccggtctggtggcaattgaaaacatcaaggctggggacaaggtgatcgccacaaatcccgaaacgtttgaggtcgccgaaaaaaccgtgttagagacttatgttcgtgaaaccacagaacttttgcatttgacgattggcggagaagtcatcaaaacgactttcgaccatcctttctatgtgaaagatgtcggtttcgtggaagcaggtaagttacaggtgggcgacaagttattagattcgcgcgggaatgttttagtcgtagaggagaaaaagttggagattgccgacaagcctgttaaagtctatgacttcaaagtggatgacttccacacctaccatgtgggagataacgaagtgttagttcacaat

SEQ ID NO: 38 - DNA, 504 bp, Artificial Sequence
  Codon-optimized sequence #1 encoding Deinococcus swuensis PolIII alpha subunit non-ligating mini-intein (SEQ ID NO8)
atggcgctgaccggggatactcgcgtgcctgttgcaggcggagaactgcgccgtatcgaggatttatgtggtgaaggccaacctgttcaattaccgtcggtgaatggagaataccgtcttgagctgcgcccgactgggcagttttttgacaacggcgtgaagccggtctttaaggtaaagaccgcgcttggtcgtgaattaaccgctacggggaaccacccacttttaacgttggacggttggtggcgcgacgtcgaagcgcaatccggggtttgtaccaaggagttctatgggacccctaaagcgcataagaaggggtttcgccgttctacgatccaaacgcttggtgagttcttcggggcgactgaactgctggatgcctgccttgatgatttatattgggacacaatcacgtccatcacaccagcgggcgaggcacaaactttcgatttggaagtgcctggtacacacaacttcgtagccaatgatctgatcgtacataac

SEQ ID NO: 39 - DNA, 504 bp, Artificial Sequence
  Codon-optimized sequence #2 encoding Deinococcus swuensis PolIII alpha subunit non-ligating mini-intein (SEQ ID NO8)
atggccttgacgggagatacccgtgttcccgttgccgggggagaattacgtcgtattgaagatctttgcggggaagggcagccagttcaattgcctagtgtgaacggcgaatatcgtttagaactgcgtcctacaggtcagttctttgataatggtgtgaaacccgtgtttaaggtaaagactgcattaggacgcgagttgactgccaccgggaatcacccgcttcttacccttgatgggtggtggcgcgatgtggaagcccaatcaggcgtatgtacgaaagagttctacggcacgcccaaggcacacaaaaaaggcttccgtcgctccacaattcaaacattaggcgaattttttggagcaaccgaactgttagatgcctgtttagatgatctgtactgggatacaatcacaagcattacacctgccggagaagcccaaacgttcgacttagaggtcccaggcactcacaattttgtagcaaacgaccttatcgtacacaat

SEQ ID NO: 40 - DNA, 489 bp, Artificial Sequence
  Codon-optimized sequence #1 encoding Cyanobacterium aponinum DnaB non-ligating intein (SEQ ID NO9)
atggcgctgtccggggattccttaattgcactggctgatactaacacggtggtgccaattcgtgaattagtgagcaagaaggacatcacagtgttcgcgttaaatgaggcgacaatgaagatcgaaaaagctaaagtcatcaatgccttctgcacgggcatcaagccagtattttgtcttaagacacgtttaggtcgtactatcaaggccaccgccaatcacaaattcttcactatcgagggttggaaacgtcttgacgaattatccgttaaagatttcatcgcgcttcctcgctatttaccctcctcggaaatctctacaattaaactgggagaaattgtgcaatgtgacgtaattaagtcgttagctaactcggatatttattgggatgaaatcacctcaatcgaaccattaggcgagacggaagtgtacgaccttactgtagagaactatcataatttcattgccaacaatatcattgttcacaac

SEQ ID NO: 41 - DNA, 489 bp, Artificial Sequence
  Codon-optimized sequence #2 encoding Cyanobacterium aponinum DnaB non-ligating intein (SEQ ID NO9)
atggcattaagcggggacagtctgattgctttggcggacacaaatacagttgtgcctattcgtgagcttgtgagtaaaaaagacatcacggtttttgctttgaacgaagctactatgaaaattgagaaagccaaggtgattaatgctttctgtacggggattaagcccgtcttttgccttaaaacccgcctgggacgcaccatcaaggccaccgcaaatcacaaattttttactattgaagggtggaagcgtttagatgaacttagcgttaaggactttattgcgctgccgcgctacttaccctcatcagaaatctcgacgattaagttaggtgaaatcgtgcagtgcgatgttatcaaatctttagcaaacagtgacatttactgggatgagattactagcatcgaacccttaggcgaaacagaagtatacgatttgactgtggaaaattaccacaatttcatcgccaataacatcattgtccacaac

SEQ ID NO: 42 - DNA, 285 bp, Artificial Sequence
  Codon-optimized sequence encoding Synechocystis sp. PCC6803 DnaX non-ligating split intein, N-terminal portion (SEQ ID NO10)
atggctcttaccggagactcgcaggtccttacacgcaacgggttaatgtctattgacaacccacagattaagggtcgtgaggtactttcgtacaatgaaacacttcaacagtgggaatataagaaagtcttacgttggttggatcgcggcgagaagcaaactctttcaatcaaaacaaaaaactcgacggttcgctgtacagccaaccacttaattcgtaccgaacaggggtggacacgtgcggaaaatatcacgccgggcatgaaaattctttcccctgccagc

SEQ ID NO: 43 - DNA, 129 bp, Artificial Sequence
  Codon-optimized sequence #1 encoding Synechocystis sp. PCC6803 DnaX non-ligating split intein, C-terminal portion (SEQ ID NO11)
atgtcgccacaatggcataccaattttgaggaggtggagtcggttacaaaaggtcaggtggaaaaggtatatgaccttgaggtagaagacaatcacaattttgttgcaaatgggcttttagttcataac

SEQ ID NO: 44 - DNA, 129 bp, Artificial Sequence
  Codon-optimized sequence #2 encoding Synechocystis sp. PCC6803 DnaX non-ligating split intein, C-terminal portion (SEQ ID NO11)
atgagtccgcagtggcatactaatttcgaggaggtagagtcagtcactaagggtcaggtggagaaggtttatgatttggaagtggaagacaatcataatttcgtggcaaatggtctgcttgttcataac

SEQ ID NO: 45 - DNA, 183 bp, Artificial Sequence
  Codon-optimized sequence #1 encoding Synechocystis sp. PCC6803 DnaX 6xHis-tagged non-ligating split intein, C-terminal portion (SEQ ID NO12)
atgcaccaccatcaccatcacgattacaaagatgatgatgacaaaggtagcggatcatcgccacaatggcataccaattttgaggaggtggagtcggttacaaaaggtcaggtggaaaaggtatatgaccttgaggtagaagacaatcacaattttgttgcaaatgggcttttagttcataac

SEQ ID NO: 46 - DNA, 183 bp, Artificial Sequence
  Codon-optimized sequence #2 encoding Synechocystis sp. PCC6803 DnaX 6xHis-tagged non-ligating split intein, C-terminal portion (SEQ ID NO12)
atgcaccaccatcaccatcacgattacaaagatgatgatgacaaaggaagtgggtccagtccgcagtggcatactaatttcgaggaggtagagtcagtcactaagggtcaggtggagaaggtttatgatttggaagtggaagacaatcataatttcgtggcaaatggtctgcttgttcataac

SEQ ID NO: 47 - DNA, 321 bp, Artificial Sequence
  Codon-optimized sequence encoding Synechocystis sp. PCC6803 DnaB non-ligating split intein, N-terminal portion (SEQ ID NO13)
atggctatttcaggcgacagtctgatctctcttgcttccacagggaagcgcgtatctattaaagatttgttggacgaaaaagattttgaaatttgggctatcaacgagcaaaccatgaagctggaatctgcaaaggtcagccgcgtcttctgcacaggtaaaaaacttgtttacattcttaaaacgcgtctgggtcgtacaatcaaggcgacagccaaccatcgctttttaaccattgatggttggaagcgtctggatgagttgtctctgaaggagcacatcgcgcttcctcgtaaactggagtcgtctagtttgcaattg

SEQ ID NO: 48 - DNA, 147 bp, Artificial Sequence
  Codon-optimized sequence #1 encoding Synechocystis sp. PCC6803 DnaB non-ligating split intein, C-terminal portion (SEQ ID NO14)
atgtcgccggagatcgaaaagttgtcccagtccgacatctactgggattcgatcgttagcatcactgagacaggagtggaagaggtgttcgacttaacggtgcctggaccccataactttgtggcgaacgatatcatcgttcacaac

SEQ ID NO: 49 - DNA, 147 bp, Artificial Sequence
  Codon-optimized sequence #2 encoding Synechocystis sp. PCC6803 DnaB non-ligating split intein, C-terminal portion (SEQ ID NO14)
atgtctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 50 - DNA, 201 bp, Artificial Sequence
  Codon-optimized sequence #1 encoding Synechocystis sp. PCC6803 DnaB 6xHis-tagged non-ligating split intein, C-terminal portion (SEQ ID NO15)
atgcaccaccatcaccatcacgattacaaagatgatgatgacaaagggtcggggtcttcgccggagatcgaaaagttgtcccagtccgacatctactgggattcgatcgttagcatcactgagacaggagtggaagaggtgttcgacttaacggtgcctggaccccataactttgtggcgaacgatatcatcgttcacaac

SEQ ID NO: 51 - DNA, 201 bp, Artificial Sequence
  Codon-optimized sequence #2 encoding Synechocystis sp. PCC6803 DnaB 6xHis-tagged non-ligating split intein, C-terminal portion (SEQ ID NO15)
atgcaccaccatcaccatcacgattacaaagatgatgatgacaaaggaagtggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 52 - DNA, 267 bp, Artificial Sequence
  Codon-optimized sequence encoding Prochlorococcus cyanophage P-SSM2 gp41-1 non-ligating split intein, N-terminal portion (SEQ ID NO16)
atggccctggatctgaagacgcaagttcagacgccgcagggtatgaaggaaatctcgaacattcaagtcggtgatttagtgcttagcaatacgggctacaacgaagtcctgaacgtatttcctaaatcaaaaaagaaatcctacaagattactttagaagacgggaaggagatcatttgtagtgaagagcacttgtttcctacccaaaccggagagatgaatatcagcggtggtcttaaggaaggtatgtgtttatacgtgaaagag

SEQ ID NO: 53 - DNA, 108 bp, Artificial Sequence
  Codon-optimized sequence #1 encoding Prochlorococcus cyanophage P-SSM2 gp41-1 non-ligating split intein, C-terminal portion (SEQ ID NO17)
atgcttaagaaaatcttgaagattgaggagttagatgagcgcgaactgattgacattgaggtgtcaggaaaccatctgttctatgccaacgacattcttacgcacaac

SEQ ID NO: 54 - DNA, 108 bp, Artificial Sequence
  Codon-optimized sequence #2 encoding Prochlorococcus cyanophage P-SSM2 gp41-1 non-ligating split intein, C-terminal portion (SEQ ID NO17)
atgttgaaaaagattttgaagatcgaagagttggacgaacgcgaattgatcgacatcgaggtatcaggcaaccatttattttatgcgaatgacatcttaacacacaac

SEQ ID NO: 55 - DNA, 282 bp, Artificial Sequence
  Codon-optimized sequence encoding Clostridium thermocellum CthBIL4 N115D non-ligating split intein ′C42′, N-terminal portion (SEQ ID NO18)
atggcttttgtagcgggcaccatgattcttacagctaccggcttagtcgccatcgaaaatatcaaggcaggcgacaaggtaatcgcgacaaatcctgagacttttgaggtcgccgagaagactgtattggaaacctatgtccgtgagacaactgaattgctgcacctgacgattggtggtgaagtgattaaaaccactttcgatcaccccttctacgtaaaggatgtaggttttgtggaggccggtaaattacaggttggggacaagctgttagattcgcgc

SEQ ID NO: 56 - DNA, 129 bp, Artificial Sequence
  Codon-optimized sequence #1 encoding Clostridium thermocellum CthBIL4 N115D non-ligating split intein ′C42′, C-terminal portion (SEQ ID NO19)
atgggtaacgttctggtggtcgaagaaaagaaacttgagattgcagacaaacccgtgaaagtctacgacttcaaggtggacgactttcacacttaccacgtcggagataatgaagtgcttgtccataac

SEQ ID NO: 57 - DNA, 129 bp, Artificial Sequence
  Codon-optimized sequence #2 encoding Clostridium thermocellum CthBIL4 N115D non-ligating split intein ′C42′, C-terminal portion (SEQ ID NO19)
atggggaatgttttagtcgtagaggagaaaaagttggagattgccgacaagcctgttaaagtctatgacttcaaagtggatgacttccacacctaccatgtgggagataacgaagtgttagttcacaat

SEQ ID NO: 58 - DNA, 360 bp, Artificial Sequence
  Codon-optimized sequence encoding Clostridium thermocellum CthBIL4 N115D non-ligating split intein ′C16′, N-terminal portion (SEQ ID NO20)
atggcttttgtagcgggcaccatgattcttacagctaccggcttagtcgccatcgaaaatatcaaggcaggcgacaaggtaatcgcgacaaatcctgagacttttgaggtcgccgagaagactgtattggaaacctatgtccgtgagacaactgaattgctgcacctgacgattggtggtgaagtgattaaaaccactttcgatcaccccttctacgtaaaggatgtaggttttgtggaggccggtaaattacaggttggggacaagctgttagattcgcgcggtaacgttctggtggtcgaagaaaagaaacttgagattgcagacaaacccgtgaaagtctacgacttcaaggtggac

SEQ ID NO: 59 - DNA, 51 bp, Artificial Sequence
  Codon-optimized sequence #1 encoding Clostridium thermocellum CthBIL4 N115D non-ligating split intein ′C16′, C-terminal portion (SEQ ID NO21)
atggactttcacacttaccacgtcggagataatgaagtgcttgtccataac

SEQ ID NO: 60 - DNA, 51 bp, Artificial Sequence
  Codon-optimized sequence #2 encoding Clostridium thermocellum CthBIL4 N115D non-ligating split intein ′C16′, C-terminal portion (SEQ ID NO21)
atggacttccacacctaccatgtgggagataacgaagtgttagttcacaat

SEQ ID NO: 61 - DNA, 507 bp, Artificial Sequence
  Codon-optimized sequence #2A encoding Synechocystis sp. PCC6803 DnaB 6xHis-tagged non-ligating mini-intein (SEQ ID NO2)
atggcaatatcgggtgattccctgatctcattggcttctaccggaaagcgcgtaagcatcaaagacctgcttgatgagaaagatttcgaaatctgggcgattaacgagcaaactatgaaattggagtctgctaaggtaagtcgcgtattctgcactgggaaaaagttagtttatattttgaaaacacgtttagggcgcactattaaggcaacagcaaatcaccgctttctgaccattgatggttggaaacgtctggatgagcttagtcttaaagaacacatcgctttgcctcgcaaacttgaatcgtcatcgcttcaactggcgtcaggccatcatcatcaccatcatgggggatcgggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 62 - DNA, 507 bp, Artificial Sequence
  Codon-optimized sequence #2B encoding Synechocystis sp. PCC6803 DnaB 6xHis-tagged non-ligating mini-intein (SEQ ID NO2)
atggcaatatcgggtgattctctgatctcgttggcttctaccggaaagcgcgtaagcatcaaagacctgcttgatgagaaagatttcgaaatctgggcgattaacgagcaaactatgaaattggagtctgctaaggtaagtcgcgtattctgcactgggaaaaagttagtttatattttgaaaacacgtttagggcgcactattaaggcaacagcaaatcaccgctttctgaccattgatggttggaaacgtctggatgagcttagtcttaaagaacacatcgctttgcctcgcaaacttgaatcgtcatcgcttcaactggcgtcaggccatcatcatcaccatcatgggggatcgggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 63 - DNA, 507 bp, Artificial Sequence
  Codon-optimized sequence #2C encoding Synechocystis sp. PCC6803 DnaB 6xHis-tagged non-ligating mini-intein (SEQ ID NO2)
atggctatctcaggtgattcgctgatttctttggcttctaccggaaagcgcgtaagcatcaaagacctgcttgatgagaaagatttcgaaatctgggcgattaacgagcaaactatgaaattggagtctgctaaggtaagtcgcgtattctgcactgggaaaaagttagtttatattttgaaaacacgtttagggcgcactattaaggcaacagcaaatcaccgctttctgaccattgatggttggaaacgtctggatgagcttagtcttaaagaacacatcgctttgcctcgcaaacttgaatcgtcatcgcttcaactggcgtcaggccatcatcatcaccatcatgggggatcgggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 64 - DNA, 507 bp, Artificial Sequence
  Codon-optimized sequence #2D encoding Synechocystis sp. PCC6803 DnaB 6xHis-tagged non-ligating mini-intein (SEQ ID NO2)
atggctatttcaggcgattcactaatttcattggcttctaccggaaagcgcgtaagcatcaaagacctgcttgatgagaaagatttcgaaatctgggcgattaacgagcaaactatgaaattggagtctgctaaggtaagtcgcgtattctgcactgggaaaaagttagtttatattttgaaaacacgtttagggcgcactattaaggcaacagcaaatcaccgctttctgaccattgatggttggaaacgtctggatgagcttagtcttaaagaacacatcgctttgcctcgcaaacttgaatcgtcatcgcttcaactggcgtcaggccatcatcatcaccatcatgggggatcgggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 65 - DNA, 507 bp, Artificial Sequence
  Codon-optimized sequence DnaB_Alt_v02 encoding Synechocystis sp. PCC6803 DnaB 6xHis-tagged non-ligating mini-intein (SEQ ID NO2)
atggctatttcgggcgattcactgataagtttggcttctaccggaaagcgcgtaagcatcaaagacctgcttgatgagaaagatttcgaaatctgggcgattaacgagcaaactatgaaattggagtctgctaaggtaagtcgcgtattctgcactgggaaaaagttagtttatattttgaaaacacgtttagggcgcactattaaggcaacagcaaatcaccgctttctgaccattgatggttggaaacgtctggatgagcttagtcttaaagaacacatcgctttgcctcgcaaacttgaatcgtcatcgcttcaactggcgtcaggccatcatcatcaccatcatgggggatcgggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 66 - DNA, 507 bp, Artificial Sequence
  Codon-optimized sequence DnaB_Alt_v03 encoding Synechocystis sp. PCC6803 DnaB 6xHis-tagged non-ligating mini-intein (SEQ ID NO2)
atggccatatctggtgactcgctgatcagtttggcttctaccggaaagcgcgtaagcatcaaagacctgcttgatgagaaagatttcgaaatctgggcgattaacgagcaaactatgaaattggagtctgctaaggtaagtcgcgtattctgcactgggaaaaagttagtttatattttgaaaacacgtttagggcgcactattaaggcaacagcaaatcaccgctttctgaccattgatggttggaaacgtctggatgagcttagtcttaaagaacacatcgctttgcctcgcaaacttgaatcgtcatcgcttcaactggcgtcaggccatcatcatcaccatcatgggggatcgggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 67 - DNA, 507 bp, Artificial Sequence
  Codon-optimized sequence DnaB_KA encoding Synechocystis sp. PCC6803 DnaB 4xHis-KA-tagged non-ligating mini-intein
atggccatttcaggtgatagtttaatctcgttagcctcaacagggaagcgcgtgtctatcaaagatctgctggatgaaaaggattttgaaatttgggctatcaatgaacaaaccatgaagttggaatccgcgaaggtaagtcgtgtcttctgtacgggaaaaaagctggtgtatatcctgaagacccgtttgggccgcactatcaaggcaaccgctaatcatcgcttcctgaccattgacggatggaagcgtttagacgaattgagcttgaaagaacatatcgctttaccgcgcaaattggaatccagtagccttcaacttgcctctggacatcatcatcataaagccggtggtagcggctcctccccagaaattgagaagctttcacaatctgacatctactgggacagtatcgtatctattaccgagacgggtgttgaagaggtgtttgatcttacggtaccaggtccgcataattttgtagctaatgacatcattgtgcacaac

SEQ ID NO: 68 - DNA, 507 bp, Artificial Sequence
  Codon-optimized sequence DnaB_KA_var2 encoding Synechocystis sp. PCC6803 DnaB 4xHis-KA-tagged non-ligating mini-intein
atggccatctcaggggactcgctgattagcctggcatccaccgggaagcgtgtttccattaaggacctgctggacgagaaagattttgaaatctgggcaatcaatgagcaaacgatgaagttagagtcggctaaagtgtcccgcgtcttttgcaccggtaaaaaattggtgtacattcttaaaacccgtcttggtcgtaccattaaagcgacagcgaaccatcgttttttaacgatcgacggctggaagcgcctggacgagttgtcactgaaagaacacattgccttaccacgcaagttagaaagttcctcattacagctggccagcggccatcaccaccacaaggccggtgggtccggatcgtcacccgaaatcgaaaaattgtctcaatcggatatctattgggatagcattgtctccatcacggaaacgggcgtcgaggaagtttttgatcttacggtgccgggaccacacaatttcgtagccaatgacatcattgtgcataac

SEQ ID NO: 69 - DNA, 507 bp, Artificial Sequence
  Codon-optimized sequence DnaB_KA_var2-1 encoding Synechocystis sp. PCC6803 DnaB 4xHis-KA-tagged non-ligating mini-intein
atggccatttcaggtgattcgcttataagtctggcatccaccgggaagcgtgtttccattaaggacctgctggacgagaaagattttgaaatctgggcaatcaatgagcaaacgatgaagttagagtcggctaaagtgtcccgcgtcttttgcaccggtaaaaaattggtgtacattcttaaaacccgtcttggtcgtaccattaaagcgacagcgaaccatcgttttttaacgatcgacggctggaagcgcctggacgagttgtcactgaaagaacacattgccttaccacgcaagttagaaagttcctcattacagctggccagcggccatcaccaccacaaggccggtgggtccggatcgtcacccgaaatcgaaaaattgtctcaatcggatatctattgggatagcattgtctccatcacggaaacgggcgtcgaggaagtttttgatcttacggtgccgggaccacacaatttcgtagccaatgacatcattgtgcataac

SEQ ID NO: 70 - DNA, 507 bp, Artificial Sequence
  Codon-optimized sequence DnaB_KA_var2-2 encoding Synechocystis sp. PCC6803 DnaB 4xHis-KA-tagged non-ligating mini-intein
atggcgatatcgggtgattctctaatcagtctcgcatccaccgggaagcgtgtttccattaaggacctgctggacgagaaagattttgaaatctgggcaatcaatgagcaaacgatgaagttagagtcggctaaagtgtcccgcgtcttttgcaccggtaaaaaattggtgtacattcttaaaacccgtcttggtcgtaccattaaagcgacagcgaaccatcgttttttaacgatcgacggctggaagcgcctggacgagttgtcactgaaagaacacattgccttaccacgcaagttagaaagttcctcattacagctggccagcggccatcaccaccacaaggccggtgggtccggatcgtcacccgaaatcgaaaaattgtctcaatcggatatctattgggatagcattgtctccatcacggaaacgggcgtcgaggaagtttttgatcttacggtgccgggaccacacaatttcgtagccaatgacatcattgtgcataac

SEQ ID NO: 71 - DNA, 507 bp, Artificial Sequence
  Codon-optimized sequence DnaB_KA_var2-3_mutant encoding Synechocystis sp. PCC6803 DnaB 4xHis-KA-tagged non-ligating mini-intein
atggccatatctggtgactcgctgatcagtcttgcatccaccgggaagcgtgtttccattaaggacctgctggacgagaaagattttgaaatctgggcaatcaatgagcaaacgatgaagttagagtcggctaaagtgtcccgcgtcttttgcaccggtaaaaaattggtgtacattcttaaaacccgtcttggtcgtgccattaaagcgacagcgaaccatcgttttttaacgatcgacggctggaagcgcctggacgagttgtcactgaaagaacacattgccttaccacgcaagttagaaagttcctcattacagctggccagcggccatcaccaccacaaggccggtgggtccggatcgtcacccgaaatcgaaaaattgtctcaatcggatatctattgggatagcattgtctccatcacggaaacgggcgtcgaggaagtttttgatcttacggtgccgggaccacacaatttcgtagccaatgacatcattgtgcataac

SEQ ID NO: 72 - DNA, 507 bp, Artificial Sequence
  Codon-optimized sequence DnaB_KA_var2-4 encoding Synechocystis sp. PCC6803 DnaB 4xHis-KA-tagged non-ligating mini-intein
atggctatatctggagattcacttatcagtctcgcatccaccgggaagcgtgtttccattaaggacctgctggacgagaaagattttgaaatctgggcaatcaatgagcaaacgatgaagttagagtcggctaaagtgtcccgcgtcttttgcaccggtaaaaaattggtgtacattcttaaaacccgtcttggtcgtaccattaaagcgacagcgaaccatcgttttttaacgatcgacggctggaagcgcctggacgagttgtcactgaaagaacacattgccttaccacgcaagttagaaagttcctcattacagctggccagcggccatcaccaccacaaggccggtgggtccggatcgtcacccgaaatcgaaaaattgtctcaatcggatatctattgggatagcattgtctccatcacggaaacgggcgtcgaggaagtttttgatcttacggtgccgggaccacacaatttcgtagccaatgacatcattgtgcataac

SEQ ID NO: 73 - DNA, 507 bp, Artificial Sequence
  Codon-optimized sequence DnaB_KA_var2-5 encoding Synechocystis sp. PCC6803 DnaB 4xHis-KA-tagged non-ligating mini-intein
atggccatttctggcgattcgctaattagtcttgcatccaccgggaagcgtgtttccattaaggacctgctggacgagaaagattttgaaatctgggcaatcaatgagcaaacgatgaagttagagtcggctaaagtgtcccgcgtcttttgcaccggtaaaaaattggtgtacattcttaaaacccgtcttggtcgtaccattaaagcgacagcgaaccatcgttttttaacgatcgacggctggaagcgcctggacgagttgtcactgaaagaacacattgccttaccacgcaagttagaaagttcctcattacagctggccagcggccatcaccaccacaaggccggtgggtccggatcgtcacccgaaatcgaaaaattgtctcaatcggatatctattgggatagcattgtctccatcacggaaacgggcgtcgaggaagtttttgatcttacggtgccgggaccacacaatttcgtagccaatgacatcattgtgcataac

SEQ ID NO: 74 - DNA, 507 bp, Artificial Sequence
  Codon-optimized sequence DnaB_KA_var2-6 encoding Synechocystis sp. PCC6803 DnaB 4xHis-KA-tagged non-ligating mini-intein
atggctatttcgggcgattcactgataagtctagcatccaccgggaagcgtgtttccattaaggacctgctggacgagaaagattttgaaatctgggcaatcaatgagcaaacgatgaagttagagtcggctaaagtgtcccgcgtcttttgcaccggtaaaaaattggtgtacattcttaaaacccgtcttggtcgtaccattaaagcgacagcgaaccatcgttttttaacgatcgacggctggaagcgcctggacgagttgtcactgaaagaacacattgccttaccacgcaagttagaaagttcctcattacagctggccagcggccatcaccaccacaaggccggtgggtccggatcgtcacccgaaatcgaaaaattgtctcaatcggatatctattgggatagcattgtctccatcacggaaacgggcgtcgaggaagtttttgatcttacggtgccgggaccacacaatttcgtagccaatgacatcattgtgcataac

SEQ ID NO: 75 - DNA, 507 bp, Artificial Sequence
  Codon-optimized sequence DnaB_KH encoding Synechocystis sp. PCC6803 DnaB HHHHKH-tagged non-ligating mini-intein
atggctattagcggtgactccttaatctctttagctagtacaggtaagcgcgtatcaattaaggacttactggatgaaaaggatttcgagatctgggcaattaacgagcagactatgaaattagaatctgccaaggtatcgcgtgttttttgcactggtaaaaagttggtttatattcttaaaactcgtctgggccgtacgattaaagccactgccaatcaccgttttcttaccattgatggctggaagcgtttggatgaactgtcacttaaagaacatattgcgcttcctcgtaaacttgaatcctcgtctttgcaacttgcatcaggtcaccatcaccacaagcatggaggctcagggagttcgcccgaaattgaaaagctgtctcagtctgatatctactgggattctattgtatccattactgaaaccggtgtggaggaagtcttcgatttaacggttcctggtccgcacaattttgtagctaatgatatcattgtccacaac

SEQ ID NO: 76 - DNA, 507 bp, Artificial Sequence
  Codon-optimized sequence DnaB_KS encoding Synechocystis sp. PCC6803 DnaB HHHHKS-tagged non-ligating mini-intein
atggcaatctctggtgacagtttgatctctttagcatcaactggcaagcgtgtatccattaaggatttgctggatgaaaaggattttgaaatctgggcgattaatgaacaaacgatgaaattagagagcgcgaaagtcagccgcgtgttttgtaccgggaagaaattggtatacatcctgaagacgcgccttggccgcactattaaggcgacagcgaatcaccgtttcttaacgatcgacggctggaagcgcctggatgaactgagcttaaaagagcacatcgccctgccacgcaagctggaatcttctagcttacaactggcgtcgggtcatcaccaccataagtcgggtggttctgggtcttcgccggaaatcgaaaaactgtcacagtccgatatttactgggactctatcgtcagtattaccgagactggcgtcgaggaggtatttgacttaacggtgcctgggcctcataactttgtggctaatgacattattgtgcataat

SEQ ID NO: 77 - DNA, 507 bp, Artificial Sequence
  Codon-optimized sequence DnaB_KT encoding Synechocystis sp. PCC6803 DnaB HHHHKT-tagged non-ligating mini-intein
atggcgatttcgggggactcgcttatttcactggcttccaccgggaagcgcgtatctatcaaggatttattggatgaaaaagacttcgagatttgggcgattaatgagcagaccatgaagcttgaaagtgcgaaagtctcccgcgtgttctgcaccggaaagaagttggtctacattcttaaaacgcgcctgggtcgtacaatcaaagcgacggctaaccatcgtttcttgaccattgatggttggaagcgtttggacgaactgagtctgaaagagcacatcgcattaccccgcaagcttgagtccagttcgttgcagcttgccagcggacaccatcatcacaaaactggaggatcaggtagtagtcctgaaatcgagaagttgtcacagtccgatatttattgggattctattgtgagtattactgaaacaggcgtagaagaagttttcgacttaacagtccctggtccacataacttcgtggccaatgacatcatcgtgcataac

SEQ ID NO: 78 - DNA, 465 bp, Artificial Sequence
  Codon-optimized sequence DnaB_M86 encoding Synechocystis sp. PCC6803 DnaB non-ligating mini-intein
atggcaattagcggagattctcttatttcgttagcatcgaccgggaagcgcgtcccaatcaaagaccttttaggagagaaagatttcgagatttgggccatcaatgagcagacgatgaagttggaaagcgcaaaagtgtctcgtgtgttttgtacgggaaagaaattagtatacacgttgaaaacgcgcttagggcgtaccatcaaagcgactgctaaccaccgctttttgaccattgacggctggaaacgtctggatgagttatcattgaaggagcatatcgctttaccgcgtaagttagagtcgtcatccttacaactggctcccgaaatcgagaaacttccacagtccgacatttactgggaccctatcgtctccatcaccgagacaggggtggaagaggtcttcgatttaaccgtcccggggttacgcaacttcgttgcgaacgacattatcgttcataac

SEQ ID NO: 79 - DNA, 507 bp, Artificial Sequence
  Codon-optimized sequence DnaB_v03 encoding Synechocystis sp. PCC6803 DnaB 6xHis-tagged non-ligating mini-intein (SEQ ID NO2)
atggcgattagcggcgatagtctgattagtctggccagcaccggcaagcgtgtgagcatcaaggatctgctggacgaaaaggacttcgagatctgggccatcaacgagcagaccatgaaactggagagcgccaaagttagccgcgttttctgcaccggtaagaagctcgtgtacatcctcaagacccgtctgggtcgtaccatcaaagcgacggcgaaccatcgctttctgaccatcgatggctggaaacgtctggacgaactgagtctgaaagagcatatcgcgctgccacgcaagctggaaagcagtagtctgcagctggcgagcggccaccatcatcaccatcatggcggcagcggtagcagcccggaaatcgagaagctgagccagagcgacatctactgggatagcatcgtgagcatcaccgagacgggcgtggaagaagtgtttgatctgacggttccgggcccgcacaacttcgtggccaacgacatcatcgtgcacaac

SEQ ID NO: 80 - DNA, 507 bp, Artificial Sequence
  Codon-optimized sequence DnaB_v04 encoding Synechocystis sp. PCC6803 DnaB 6xHis-tagged non-ligating mini-intein (SEQ ID NO2)
atggcgatcagcggcgatagtctgatcagtctggcgagtaccggcaaacgcgtgagcatcaaggatctgctcgatgagaaggacttcgagatctgggcgatcaacgagcagaccatgaagctggagagcgcgaaagtgagtcgcgttttctgcacgggcaagaagctggtgtacatcctcaaaacccgtctcggccgcaccattaaggcgaccgccaatcaccgctttctgaccattgatggctggaaacgtctggatgaactgagtctgaaggaacacatcgcgctgccacgcaaactggagagtagcagcctccagctggccagcggtcatcatcaccaccatcatggtggtagcggtagcagcccggaaattgagaagctcagccagagcgacatctactgggatagcatcgtgagcatcaccgaaacgggtgtggaagaagttttcgatctgacggttccgggcccgcacaactttgtggcgaacgatatcatcgtgcacaac

SEQ ID NO: 81 - DNA, 507 bp, Artificial Sequence
  Codon-optimized sequence DnaB_v05 encoding Synechocystis sp. PCC6803 DnaB 6xHis-tagged non-ligating mini-intein (SEQ ID NO2)
atggctatttcaggagatagtctaatatctttggctagcaccggcaagcgcgtatccatcaaagaccttctggacgagaaggacttcgagatctgggcaattaacgaacaaaccatgaaattggagagcgcgaaagttagtcgtgtgttttgtaccggtaagaagttggtgtacatcctgaaaacccgtctgggccgtaccattaaagctaccgcgaaccaccgctttctgaccattgatggttggaaacgtttggatgagctgagtctgaaagagcacattgcgttaccgcgtaaacttgagagcagctccctgcaattggctagcggtcatcaccaccaccaccatggtggttccggctcatccccggagattgaaaaactgagccagagcgatatttactgggatagcatcgtgtctatcaccgaaaccggtgtcgaggaagttttcgacctgacggtgccgggtccgcataactttgttgcgaatgacatcatcgttcacaac

SEQ ID NO: 82 - DNA, 507 bp, Artificial Sequence
  Codon-optimized sequence DnaB_v06 encoding Synechocystis sp. PCC6803 DnaB 6xHis-tagged non-ligating mini-intein (SEQ ID NO2)
atggctatttcaggagatagtctaatatctctcgcgagcaccggcaagcgcgtgtccatcaaagacttgcttgacgaaaaggactttgagatctgggcaattaacgaacaaaccatgaaacttgagagcgctaaagtgtcacgtgtgttctgcaccggtaagaaattggtgtacatcctgaaaacgcgtctgggtcgtaccatcaaggcgacggcgaatcatcgctttctgaccatcgatggttggaagagactggatgagctgagcttaaaggagcacattgcactgccgcgtaaattggaaagctcttccctgcaattggcgagcggccaccaccatcaccaccacggcggatctggctcgtccccggagatcgagaagttaagccagtcggacatctactgggatagcatcgtgtctatcaccgagacgggtgtcgaggaagtttttgatttgacggttccgggtccgcataactttgtggcgaatgatattatcgtgcataat

SEQ ID NO: 83 - DNA, 507 bp, Artificial Sequence
  Codon-optimized sequence DnaB_v07 encoding Synechocystis sp. PCC6803 DnaB 6xHis-tagged non-ligating mini-intein (SEQ ID NO2)
atggcaatctctggtgactctctgatttctctggctagcacgggtaaacgcgtttcgatcaaagatcttctggatgagaaagatttcgagatctgggcgattaacgaacagaccatgaaattagagagcgccaaggttagccgcgtcttttgcacgggtaagaaactggtgtatatcttgaaaacgcgtctgggccgcactattaaggccaccgccaaccatcgcttcctgaccattgacggttggaagcgcttggatgaactgagcctgaaagaacacatcgcgctgccgcgtaaactggagtccagctccttgcaactggcgagcggtcatcatcaccaccatcatggtggcagcggtagcagcccagagatcgaaaagttgagccagagcgacatttactgggactccattgttagcattaccgaaacgggcgtcgaggaagtttttgatctgaccgtgccaggtccgcacaattttgtcgcaaacgatatcatcgttcacaat

SEQ ID NO: 84 - DNA, 507 bp, Artificial Sequence
  Codon-optimized sequence DnaB_v08 encoding Synechocystis sp. PCC6803 DnaB 6xHis-tagged non-ligating mini-intein (SEQ ID NO2)
atggcgatctctggcgactctctgatttctctggctagcactggtaagcgtgtttcgattaaagacttgctggatgagaaagattttgagatctgggcaatcaacgaacagactatgaaattggagtcggccaaggtcagccgcgtgttttgcaccggtaagaaactggtttacatcctgaaaacgcgtctgggtcgtacgattaaggcaacggcgaatcaccgcttcctgaccatcgacggctggaagcgcttagacgaactgagcctgaaagaacatattgcgctgccgcgtaaactggagagcagcagcctgcagctggcgagcggtcaccaccaccatcaccatggcggctctggcagcagcccggaaattgaaaagctgagccaatccgatatctactgggatagcatcgtgagcattaccgaaaccggcgtcgaggaagttttcgacctgaccgttccgggtccgcataactttgttgcgaatgacattatcgtgcataat

SEQ ID NO: 85 - DNA, 507 bp, Artificial Sequence
  Codon-optimized sequence DnaB_v09 encoding Synechocystis sp. PCC6803 DnaB 6xHis-tagged non-ligating mini-intein (SEQ ID NO2)
atggccatcagcggcgatagcctcatcagtctggcgagcaccggtaagcgtgtgagcatcaaggatctgctggacgagaaggacttcgagatttgggccatcaacgagcagaccatgaaactcgagagcgccaaggtgagccgcgttttctgcacgggcaagaagctcgtgtacattctgaaaacccgtctgggtcgcacgatcaaagcgacggcgaatcatcgctttctgaccatcgatggctggaaacgtctggatgaactcagtctgaaggagcacatcgcgctgccgcgcaaactggaaagcagcagtctgcagctggcgagcggtcatcatcatcaccatcatggcggcagcggcagcagtccggaaatcgagaaactgagccagagcgacatctactgggacagcatcgtgagcatcaccgaaaccggcgtggaagaagtgtttgatctgaccgtgccgggtccgcacaacttcgttgcgaacgacatcatcgtgcacaac

SEQ ID NO: 86 - DNA, 507 bp, Artificial Sequence
  Codon-optimized sequence DnaB_v10 encoding Synechocystis sp. PCC6803 DnaB 6xHis-tagged non-ligating mini-intein (SEQ ID NO2)
atggctatttcaggagatagtctaatatctttggctagcaccggcaaacgtgtttcgatcaaggacctcctggacgagaaggacttcgagatctgggcaattaatgaacaaaccatgaaactagagtctgcgaaagtttctcgtgttttctgcaccggcaagaagctggtttacatcctgaaaacccgtctgggtcgtaccattaaggctaccgccaaccaccgcttcctgacaatcgacggctggaagcgcctggatgaactgtccctgaaagaacatattgcactgccgcgtaaactagagtctagctcattacaactggcgtcaggtcatcaccaccaccaccacggcggtagcggcagtagcccggaaattgaaaagctgtctcagagcgatatctactgggattccatcgtgagcatcaccgaaaccggtgtggaggaagtttttgatcttacggtgccgggtccgcataattttgtggcgaacgacattattgtgcataac

SEQ ID NO: 87 - DNA, 507 bp, Artificial Sequence
  Codon-optimized sequence DnaB_v11 encoding Synechocystis sp. PCC6803 DnaB 6xHis-tagged non-ligating mini-intein (SEQ ID NO2)
atggcaatttctggtgactccctgatctctctggcttctacgggcaagcgtgtttcgattaaagatttactggatgagaaagacttcgagatttgggcgattaacgaacagaccatgaaactggagagcgcgaaagttagccgtgtgttttgtaccggtaagaaactggtctatattctgaaaacccgtctgggccgtaccattaaagcgaccgcgaatcatcgcttcctgaccatcgatggctggaagcgtttggacgaactgagcctgaaagaacacattgcactgccgcgcaagcttgagtcgtcctccctgcaactggccagcggccaccaccatcatcaccacggtggcagcggcagcagcccggaaattgaaaagctgtcgcaaagcgacatctactgggattccatcgttagcatcacggaaacgggtgtagaggaagtgtttgatctgactgtgccgggtccgcacaatttcgtcgcgaacgatatcatcgtgcataat

SEQ ID NO: 88 - DNA, 507 bp, Artificial Sequence
  Codon-optimized sequence DnaB_v12 encoding Synechocystis sp. PCC6803 DnaB 6xHis-tagged non-ligating mini-intein (SEQ ID NO2)
atggccatcagcggtgatagtctcattagtctggcgagtaccggtaagcgcgtgagtattaaggatctgctggacgagaaggactttgagatttgggcgatcaacgagcagacgatgaaactggagagcgcgaaggttagtcgtgtgttctgcaccggtaagaagctggtgtacatcctcaagacccgcctcggccgcacgattaaagccacggcgaatcaccgttttctgaccatcgatggctggaaacgtctggatgagctgagtctgaaagaacatatcgcgctgccacgcaagctggaaagcagcagtctgcagctggccagcggccatcatcaccatcatcatggtggcagcggcagcagcccagagatcgagaagctgagccagagcgacatctactgggatagcatcgttagcattaccgagacgggcgttgaagaagtgttcgatctcaccgtgccgggcccgcataatttcgtggccaatgacatcatcgtgcacaat

SEQ ID NO: 89 - DNA, 507 bp, Artificial Sequence
  Codon-optimized sequence DnaB_v13 encoding Synechocystis sp. PCC6803 DnaB 6xHis-tagged non-ligating mini-intein (SEQ ID NO2)
atggctatttcaggagatagtctaatatctttggcgtctacgggcaaacgcgtgtctatcaaggatttgctggacgagaaagacttcgaaatctgggcgattaacgagcaaacgatgaaactggaaagcgcgaaagtttctcgcgtgttttgtaccggcaagaaactggtgtatatcctgaaaacccgtctgggtcgcaccatcaaggccaccgcgaaccaccgtttcctgacgatcgacggctggaaacgcctggacgagctcagccttaaggagcacattgcgttaccgcgtaaattggagagcagctctctgcaactggcgagcggtcatcaccaccaccaccatggtggctctggctccagcccggagatcgaaaaattgtctcagagcgatatttattgggatagcatcgttagcatcaccgaaaccggtgttgaagaagtctttgatctgaccgttccgggtccgcataacttcgtggcgaatgacatcattgttcacaac

SEQ ID NO: 90 - DNA, 507 bp, Artificial Sequence
  Codon-optimized sequence DnaB_v14 encoding Synechocystis sp. PCC6803 DnaB 6xHis-tagged non-ligating mini-intein (SEQ ID NO2)
atggcaatttccggtgactctctgatctctctggcatctactggcaaacgcgtatcgatcaaagatttgctggacgagaaagattttgagatctgggcgattaacgaacagaccatgaaactggagagcgcaaaagttagccgcgtgttctgcaccggcaagaaactggtttacattctgaaaacgcgtctgggtcgcaccatcaaggcaaccgctaatcaccgtttcctgactatcgacggctggaagagactggatgaactgagcttgaaagaacacatcgcgctgccgcgtaaattggagagcagcagcctgcaactggcatccggtcatcaccatcaccaccacggtggcagcggcagcagcccggaaatcgaaaagctgtcacagtctgatatctactgggatagcatcgtcagcattaccgagactggtgtggaagaggttttcgacctgacggtcccgggtccgcacaactttgtagcgaacgacattattgttcacaat

SEQ ID NO: 91 - DNA, 507 bp, Artificial Sequence
  Codon-optimized sequence DnaB_v15 encoding Synechocystis sp. PCC6803 DnaB 6xHis-tagged non-ligating mini-intein (SEQ ID NO2)
atggcgatctctggcgactctctgatttctctggcatctactggcaagcgtgtctcgatcaaagacttgttggacgagaaagatttcgagatttgggcgattaatgaacagaccatgaaactggagagcgcaaaggttagccgtgtcttttgcaccggtaagaaactggtctatatcctgaaaacccgcctgggtcgtaccatcaaggccactgccaaccaccgtttcctgaccattgacggttggaaacgtctggatgaactgagcctgaaagaacacattgcgctgccgcgcaaattagagagcagcagcctgcagttggcgtcgggtcatcatcatcaccaccacggtggcagcggtagctccccggaaatcgagaaactgagccaaagcgatatctattgggacagcatcgttagcattaccgaaacgggtgttgaggaagtgttcgatttgaccgtcccgggtccgcataactttgtggcgaatgacatcattgtccacaac

SEQ ID NO: 92 - DNA, 507 bp, Artificial Sequence
  Codon-optimized sequence DnaB_v16 encoding Synechocystis sp. PCC6803 DnaB 6xHis-tagged non-ligating mini-intein (SEQ ID NO2)
atggcaatctctggtgactccctgatttctctggcatctactggcaagcgcgttagcatcaaagacctgttggacgagaaagatttcgagatctgggcgatcaatgaacaaaccatgaaattggagagcgctaaggtcagccgtgttttctgtaccggtaagaaactggtgtacatcctgaaaacgcgtctgggccgtacgattaaggcaacggcgaaccatcgttttctgacgatcgatggctggaagcgtctggatgaactgagccttaaagaacatattgccctgccgcgcaaactggagagcagcagcctgcaactggcgagcggtcaccaccatcaccatcacggtggctcaggtagcagccctgagattgaaaagctgagccagagcgacatctactgggatagcattgtgagcattaccgaaaccggtgtggaagaggttttcgatttgactgtgccgggtccacacaacttcgtcgcaaacgacatcatcgtccataat

SEQ ID NO: 93 - DNA, 507 bp, Artificial Sequence
  Codon-optimized sequence DnaB_v17 encoding Synechocystis sp. PCC6803 DnaB 6xHis-tagged non-ligating mini-intein (SEQ ID NO2)
atggcaatttctggtgattctctgatcagcctggcgtcgactggtaagcgtgtttctatcaaagatctgctggatgagaaagattttgagatctgggccatcaatgaacaaaccatgaaactggagagcgcaaaggtcagccgtgtgttttgtaccggcaagaaactggtttacattctgaaaacgcgcctgggccgtaccattaaagcgacggcaaaccaccgcttcctgaccatcgacggttggaagcgcctggacgaattgagcctgaaagaacacatcgcgttgccacgtaaactggagtcgtcaagcttacagttggcgtccggccaccatcatcaccaccacggtggcagcggtagcagccctgagattgaaaagctgagccaaagcgacatttactgggattccatcgtgagcattacggaaaccggtgtcgaggaagtgttcgacctgacggtcccgggtccgcacaattttgtggctaacgacatcattgtacataat

SEQ ID NO: 94 - DNA, 507 bp, Artificial Sequence
  Codon-optimized sequence DnaB_v18 encoding Synechocystis sp. PCC6803 DnaB 6xHis-tagged non-ligating mini-intein (SEQ ID NO2)
atggctatttctggtgactctctgatttctctggcatccaccggcaaacgtgtttcgatcaaagatttgctggatgagaaagattttgagatttgggcgatcaacgaacagaccatgaaactggagagcgcgaaagtttcccgtgtgttctgcaccggtaagaaactggtgtatatcttaaagacccgtctgggtcgcaccattaaggcaaccgcgaatcatcgcttcttgactatcgacggttggaaacgtctggatgaactgtcgctgaaagaacacattgctctgccgcgcaaactggagtcctctagcctgcagctggcgagcggtcatcatcaccaccaccacggcggcagcggtagctccccagagatcgagaaactgtcccagagcgacatctattgggacagcatcgtcagcatcactgaaacgggtgttgaagaggttttcgacctgacggtgccgggtccgcataattttgtggcgaatgacattatcgttcacaac

SEQ ID NO: 95 - DNA, 507 bp, Artificial Sequence
  Codon-optimized sequence DnaB_v19 encoding Synechocystis sp. PCC6803 DnaB 6xHis-tagged non-ligating mini-intein (SEQ ID NO2)
atggccatcagcggcgatagcctcatcagtctggccagtacgggtaagcgtgtgagcattaaggatctgctggatgagaaggacttcgagatctgggcgatcaacgaacagacgatgaagctggagagtgccaaggtgagccgcgtgttctgtacgggcaagaagctggtgtacattctgaaaacccgcctcggccgtaccatcaaagccaccgcgaatcaccgtttcctcaccatcgatggttggaaacgtctggacgaactgagtctgaaagaacacattgcgctgccacgcaagctggaaagcagcagtctccagctggcgagtggtcaccaccatcaccaccatggtggtagcggcagtagcccagagattgagaagctgagccagagcgatatctactgggatagcatcgtgagcatcacggaaacgggcgtggaagaggtttttgatctgaccgtgccgggcccgcacaatttcgtggcgaacgacatcatcgtgcacaac

SEQ ID NO: 96 - DNA, 507 bp, Artificial Sequence
  Codon-optimized sequence DnaB_v20 encoding Synechocystis sp. PCC6803 DnaB 6xHis-tagged non-ligating mini-intein (SEQ ID NO2)
atggcgattagcggcgatagtctgatcagtctggccagcaccggtaaacgcgtgagcatcaaagatctgctggacgaaaaggactttgagatctgggccatcaacgagcagacgatgaagctggaaagcgcgaaagtgagtcgcgtgttctgtacgggcaagaagctcgtgtacattctgaaaacccgtctgggtcgcaccattaaagcgaccgcgaatcaccgctttctgaccattgatggttggaaacgtctggatgagctgagtctgaaagaacacatcgcgctgccacgcaaactggaaagcagtagtctgcagctggcgagcggccaccatcaccaccaccatggtggcagcggcagcagcccggaaatcgagaagctcagccagagcgacatctactgggacagcatcgtgagtatcaccgagacgggcgtggaggaagtgtttgatctgaccgttccgggcccgcacaacttcgtggccaacgacatcatcgtgcacaac

SEQ ID NO: 97 - DNA, 507 bp, Artificial Sequence
  Codon-optimized sequence DnaB_v21 encoding Synechocystis sp. PCC6803 DnaB 6xHis-tagged non-ligating mini-intein (SEQ ID NO2)
atggccatcagcggcgatagtctgatcagtctggcgagcacgggtaaacgcgttagcatcaaagatctgctggacgagaaggacttcgaaatctgggcgatcaacgagcagacgatgaaactggagagcgccaaggttagccgtgtgttttgcacgggcaagaagctggtgtacatcctcaaaacgcgcctcggtcgtaccattaaagccaccgcgaaccaccgctttctcaccatcgatggctggaaacgtctggatgagctcagtctgaaagaacacatcgccctcccacgtaaactggaaagcagtagtctgcagctggccagtggccaccatcatcaccatcacggcggtagcggcagtagcccagagatcgagaagctgagccagagcgacatctactgggacagcatcgtgagcatcacggaaaccggtgttgaagaagttttcgatctgacggtgccgggcccgcataacttcgtggccaacgatatcatcgtgcacaac

SEQ ID NO: 98 - DNA, 507 bp, Artificial Sequence
  Codon-optimized sequence DnaB_v22 encoding Synechocystis sp. PCC6803 DnaB 6xHis-tagged non-ligating mini-intein (SEQ ID NO2)
atggcgatcagtggcgatagtctgatcagtctcgcgagcaccggcaaacgcgtgagcatcaaggatctgctggacgagaaagacttcgaaatttgggccatcaacgagcagaccatgaaactggagagcgccaaggttagccgcgtgttctgcaccggtaagaagctggtgtacatcctcaaaacccgtctgggccgcacgatcaaagcgaccgcgaaccatcgcttcctcacgatcgatggctggaaacgcctcgacgaactcagtctgaaagaacacattgcgctgccgcgtaaactcgaaagtagcagtctgcagctggcgagtggccatcatcaccatcatcacggcggtagcggcagcagtccagaaatcgagaaactgagccagagcgacatctactgggacagcatcgtgagcatcaccgaaacgggcgttgaggaagtgtttgatctcaccgtgccgggtccacacaactttgtggcgaacgatatcattgtgcacaac

SEQ ID NO: 99 - DNA, 507 bp, Artificial Sequence
  Codon-optimized sequence DnaB_v23 encoding Synechocystis sp. PCC6803 DnaB 6xHis-tagged non-ligating mini-intein (SEQ ID NO2)
atggctatttcaggagatagtctaatatctctggcatccaccggtaagcgcgtttccatcaaagacttgctggatgagaaagatttcgaaatttgggcaattaacgaacagaccatgaaattggagagcgctaaggttagccgcgtgttttgtaccggcaagaagttagtttacatcctgaaaacccgtctgggccgtaccattaaggcgaccgcgaaccatcgttttcttaccatcgatggttggaaacgtctcgacgagctgagcctgaaagaacatattgctctgccccgtaaattggagtcgagcagcttgcaactggcctccggtcatcaccaccaccaccacggtggcagcggctcttcgccggagatcgaaaaactgtcccagtccgacatttactgggacagcattgtatcaatcaccgaaaccggtgttgaggaagttttcgatttgacggtcccgggtccgcataactttgttgcgaatgatatcatcgtgcacaat

SEQ ID NO: 100 - DNA, 507 bp, Artificial Sequence
  Codon-optimized sequence DnaB_v24 encoding Synechocystis sp. PCC6803 DnaB 6xHis-tagged non-ligating mini-intein (SEQ ID NO2)
atggctatatctggagatagtctaatttcattggcgagcacaggcaagcgcgtgagcatcaaggaccttttggacgagaaggacttcgaaatctgggcaattaacgaacaaaccatgaaattggagtctgctaaagtgagccgtgtgttctgcaccggtaagaaattggtttacattctgaaaacccgtctgggtcgtacgattaaggcgaccgcgaaccatcgttttctgaccatcgatggctggaagcgcctggacgagctctctctgaaagaacacatcgcactgccgcgtaaattagagagcagcagcctccaactggcgagcggccaccatcaccaccaccatggtggcagcggcagcagcccggaaattgaaaaactgtctcaatccgatatctactgggactcgattgttagcatcaccgaaaccggtgtcgaagaggttttcgacctgacggtgccgggtccgcacaacttcgtggccaacgacatcatcgtgcataat

SEQ ID NO: 101 - DNA, 507 bp, Artificial Sequence
  Codon-optimized sequence DnaB_v25 encoding Synechocystis sp. PCC6803 DnaB 6xHis-tagged non-ligating mini-intein (SEQ ID NO2)
atggctatatctggagatagtctaatttcattggcgtcaacgggcaaacgcgtgtcgatcaaggacctcctggacgagaaggacttcgagatctgggcaattaatgaacagaccatgaaacttgagtctgcgaaggtgagccgcgtgttctgcaccggcaagaaactagtgtacatcctgaaaacccgtctgggtcgtaccattaaagcgactgccaaccaccgtttcttaaccattgatggttggaagcgcctggacgaactgagcctgaaagagcacattgctctgccgcgtaaattggaaagcagcagcctgcaattggcttctggtcatcaccaccaccaccacggcggcagcggttcgtccccggaaatcgaaaaactgagccaatccgacatctactgggattctatcgtgtccattaccgaaaccggtgtggaggaggtgttcgacctgacggttccgggtccgcataatttcgtggcgaacgatatcattgttcataac

SEQ ID NO: 102 - DNA, 507 bp, Artificial Sequence
  Codon-optimized sequence DnaB_v26 encoding Synechocystis sp. PCC6803 DnaB 6xHis-tagged non-ligating mini-intein (SEQ ID NO2)
atggctatatctggagatagtctaatttcactagcgtccaccggtaagcgcgtgtccatcaaagacctgctggacgagaaggactttgaaatctgggcaattaatgagcagaccatgaaactggaatccgcgaaagtgtcgcgcgtgttctgcactggtaagaagttggtctacatcctgaaaacccgtctgggccgtaccatcaaagcgaccgccaatcatcgttttctgacgattgatggttggaaacgtttggacgaactgtccctgaaagagcacattgcattgccgcgtaaacttgagagctctagcttacagctggccagcggccatcaccaccaccaccatggtggtagcggctcatccccggagatcgagaagctgtcgcaaagcgacatctattgggatagcatcgtgagcatcaccgaaaccggtgtagaggaagtattcgatctgaccgtcccgggtccgcataactttgttgcaaatgatattattgttcataac

SEQ ID NO: 103 - DNA, 507 bp, Artificial Sequence
  Codon-optimized sequence DnaB_v27 encoding Synechocystis sp. PCC6803 DnaB 6xHis-tagged non-ligating mini-intein (SEQ ID NO2)
  misc_feature (6)..(6): n is a, c, g, or t
  misc_feature (12)..(12): n is a, c, g, or t
  misc_feature (15)..(15): n is a, c, g, or t
  misc_feature (24)..(24): n is a, c, g, or t
  misc_feature (30)..(30): n is a, c, g, or t
atggcnathtcnggngayagyctnathtcncttgcttccacagggaagcgcgtatctattaaagatttgttggacgaaaaagattttgaaatttgggctatcaacgagcaaaccatgaagctggaatctgcaaaggtcagccgcgtcttctgcacaggtaaaaaacttgtttacattcttaaaacgcgtctgggtcgtacaatcaaggcgacagccaaccatcgctttttaaccattgatggttggaagcgtctggatgagttgtctctgaaggagcacatcgcgcttcctcgtaaactggagtcgtctagtttgcaattggcctccgggcaccatcaccatcatcatggggggtcggggtcttcgccggagatcgaaaagttgtcccagtccgacatctactgggattcgatcgttagcatcactgagacaggagtggaagaggtgttcgacttaacggtgcctggaccccataactttgtggcgaacgatatcatcgttcacaac

SEQ ID NO: 104 - DNA, 507 bp, Artificial Sequence
  Codon-optimized sequence DnaB_v28 encoding Synechocystis sp. PCC6803 DnaB 6xHis-tagged non-ligating mini-intein (SEQ ID NO2)
atggccatatctggtgactcgctgatcagtcttgcttccacagggaagcgcgtatctattaaagatttgttggacgaaaaagattttgaaatttgggctatcaacgagcaaaccatgaagctggaatctgcaaaggtcagccgcgtcttctgcacaggtaaaaaacttgtttacattcttaaaacgcgtctgggtcgtacaatcaaggcgacagccaaccatcgctttttaaccattgatggttggaagcgtctggatgagttgtctctgaaggagcacatcgcgcttcctcgtaaactggagtcgtctagtttgcaattggcctccgggcaccatcaccatcatcatggggggtcggggtcttcgccggagatcgaaaagttgtcccagtccgacatctactgggattcgatcgttagcatcactgagacaggagtggaagaggtgttcgacttaacggtgcctggaccccataactttgtggcgaacgatatcatcgttcacaac

SEQ ID NO: 105 - DNA, 507 bp, Artificial Sequence
  Codon-optimized sequence DnaB_v29 encoding Synechocystis sp. PCC6803 DnaB 6xHis-tagged non-ligating mini-intein (SEQ ID NO2)
atggctatttcgggcgattcactgataagtcttgcttccacagggaagcgcgtatctattaaagatttgttggacgaaaaagattttgaaatttgggctatcaacgagcaaaccatgaagctggaatctgcaaaggtcagccgcgtcttctgcacaggtaaaaaacttgtttacattcttaaaacgcgtctgggtcgtacaatcaaggcgacagccaaccatcgctttttaaccattgatggttggaagcgtctggatgagttgtctctgaaggagcacatcgcgcttcctcgtaaactggagtcgtctagtttgcaattggcctccgggcaccatcaccatcatcatggggggtcggggtcttcgccggagatcgaaaagttgtcccagtccgacatctactgggattcgatcgttagcatcactgagacaggagtggaagaggtgttcgacttaacggtgcctggaccccataactttgtggcgaacgatatcatcgttcacaac

SEQ ID NO: 106 - DNA, 507 bp, Artificial Sequence
  Codon-optimized sequence DnaB_v30 encoding Synechocystis sp. PCC6803 DnaB 6xHis-tagged non-ligating mini-intein (SEQ ID NO2)
  misc_feature (6)..(6): n is a, c, g, or t
  misc_feature (15)..(15): n is a, c, g, or t
  misc_feature (21)..(21): n is a, c, g, or t
  misc_feature (24)..(24): n is a, c, g, or t
  misc_feature (30)..(30): n is a, c, g, or t
atggcnathagyggngaytcnctnathtcnttggcttctaccggaaagcgcgtaagcatcaaagacctgcttgatgagaaagatttcgaaatctgggcgattaacgagcaaactatgaaattggagtctgctaaggtaagtcgcgtattctgcactgggaaaaagttagtttatattttgaaaacacgtttagggcgcactattaaggcaacagcaaatcaccgctttctgaccattgatggttggaaacgtctggatgagcttagtcttaaagaacacatcgctttgcctcgcaaacttgaatcgtcatcgcttcaactggcgtcaggccatcatcatcaccatcatgggggatcgggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 107 - DNA, 507 bp, Artificial Sequence; identical to SEQ ID NO: 103
  Codon-optimized sequence DnaB_v31 encoding Synechocystis sp. PCC6803 DnaB 6xHis-tagged non-ligating mini-intein (SEQ ID NO2)
  misc_feature (6)..(6): n is a, c, g, or t
  misc_feature (12)..(12): n is a, c, g, or t
  misc_feature (15)..(15): n is a, c, g, or t
  misc_feature (24)..(24): n is a, c, g, or t
  misc_feature (30)..(30): n is a, c, g, or t

SEQ ID NO: 108 - DNA, 507 bp, Artificial Sequence
  Codon-optimized sequence DnaB_v32encoding Synechocystis sp. PCC6803 DnaB 6xHis-tagged non-ligating mini-intein (SEQ ID NO2)
atggcaatatctggtgattcactcatctccttggcttctaccggaaagcgcgtaagcatcaaagacctgcttgatgagaaagatttcgaaatctgggcgattaacgagcaaactatgaaattggagtctgctaaggtaagtcgcgtattctgcactgggaaaaagttagtttatattttgaaaacacgtttagggcgcactattaaggcaacagcaaatcaccgctttctgaccattgatggttggaaacgtctggatgagcttagtcttaaagaacacatcgctttgcctcgcaaacttgaatcgtcatcgcttcaactggcgtcaggccatcatcatcaccatcatgggggatcgggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 109 - DNA, 507 bp, Artificial Sequence
  Codon-optimized sequence DnaB_v33 encoding Synechocystis sp. PCC6803 DnaB 6xHis-tagged non-ligating mini-intein (SEQ ID NO2)
atggcaatctcgggagattcactcatttcattggcttctaccggaaagcgcgtaagcatcaaagacctgcttgatgagaaagatttcgaaatctgggcgattaacgagcaaactatgaaattggagtctgctaaggtaagtcgcgtattctgcactgggaaaaagttagtttatattttgaaaacacgtttagggcgcactattaaggcaacagcaaatcaccgctttctgaccattgatggttggaaacgtctggatgagcttagtcttaaagaacacatcgctttgcctcgcaaacttgaatcgtcatcgcttcaactggcgtcaggccatcatcatcaccatcatgggggatcgggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 110 - DNA, 507 bp, Artificial Sequence
  Codon-optimized sequence DnaB_v34 encoding Synechocystis sp. PCC6803 DnaB 6xHis-tagged non-ligating mini-intein (SEQ ID NO2)
atggccatttctggtgacagtctcatttctctagcgtccaccggtaagcgcgtgtccatcaaagacctgctggacgagaaggactttgaaatctgggcaattaatgagcagaccatgaaactggaatccgcgaaagtgtcgcgcgtgttctgcactggtaagaagttggtctacatcctgaaaacccgtctgggccgtaccatcaaagcgaccgccaatcatcgttttctgacgattgatggttggaaacgtttggacgaactgtccctgaaagagcacattgcattgccgcgtaaacttgagagctctagcttacagctggccagcggccatcaccaccaccaccatggtggtagcggctcatccccggagatcgagaagctgtcgcaaagcgacatctattgggatagcatcgtgagcatcaccgaaaccggtgtagaggaagtattcgatctgaccgtcccgggtccgcataactttgttgcaaatgatattattgttcataac

SEQ ID NO: 111 - DNA, 507 bp, Artificial Sequence
  Codon-optimized sequence DnaB_v35 encoding Synechocystis sp. PCC6803 DnaB 6xHis-tagged non-ligating mini-intein (SEQ ID NO2)
atggctatctctggggatagtctaatatctctagcgtccaccggtaagcgcgtgtccatcaaagacctgctggacgagaaggactttgaaatctgggcaattaatgagcagaccatgaaactggaatccgcgaaagtgtcgcgcgtgttctgcactggtaagaagttggtctacatcctgaaaacccgtctgggccgtaccatcaaagcgaccgccaatcatcgttttctgacgattgatggttggaaacgtttggacgaactgtccctgaaagagcacattgcattgccgcgtaaacttgagagctctagcttacagctggccagcggccatcaccaccaccaccatggtggtagcggctcatccccggagatcgagaagctgtcgcaaagcgacatctattgggatagcatcgtgagcatcaccgaaaccggtgtagaggaagtattcgatctgaccgtcccgggtccgcataactttgttgcaaatgatattattgttcataac

SEQ ID NO: 112 - DNA, 507 bp, Artificial Sequence
  Codon-optimized sequence DnaB_v36 encoding Synechocystis sp. PCC6803 DnaB 6xHis-tagged non-ligating mini-intein (SEQ ID NO2)
atggcaatatcaggagatagtctaatctctctagcgtccaccggtaagcgcgtgtccatcaaagacctgctggacgagaaggactttgaaatctgggcaattaatgagcagaccatgaaactggaatccgcgaaagtgtcgcgcgtgttctgcactggtaagaagttggtctacatcctgaaaacccgtctgggccgtaccatcaaagcgaccgccaatcatcgttttctgacgattgatggttggaaacgtttggacgaactgtccctgaaagagcacattgcattgccgcgtaaacttgagagctctagcttacagctggccagcggccatcaccaccaccaccatggtggtagcggctcatccccggagatcgagaagctgtcgcaaagcgacatctattgggatagcatcgtgagcatcaccgaaaccggtgtagaggaagtattcgatctgaccgtcccgggtccgcataactttgttgcaaatgatattattgttcataac

SEQ ID NO: 113 - DNA, 507 bp, Artificial Sequence
  Codon-optimized sequence DnaB_v37 encoding Synechocystis sp. PCC6803 DnaB 6xHis-tagged non-ligating mini-intein (SEQ ID NO2)
atggctatttctggtgacagtcttatttctctagcgtccaccggtaagcgcgtgtccatcaaagacctgctggacgagaaggactttgaaatctgggcaattaatgagcagaccatgaaactggaatccgcgaaagtgtcgcgcgtgttctgcactggtaagaagttggtctacatcctgaaaacccgtctgggccgtaccatcaaagcgaccgccaatcatcgttttctgacgattgatggttggaaacgtttggacgaactgtccctgaaagagcacattgcattgccgcgtaaacttgagagctctagcttacagctggccagcggccatcaccaccaccaccatggtggtagcggctcatccccggagatcgagaagctgtcgcaaagcgacatctattgggatagcatcgtgagcatcaccgaaaccggtgtagaggaagtattcgatctgaccgtcccgggtccgcataactttgttgcaaatgatattattgttcataac

SEQ ID NO: 114 - DNA, 507 bp, Artificial Sequence
  Codon-optimized sequence DnaB_v38 encoding Synechocystis sp. PCC6803 DnaB 6xHis-tagged non-ligating mini-intein (SEQ ID NO2)
atggctatttccggagatagtctaatctcgctagcgtccaccggtaagcgcgtgtccatcaaagacctgctggacgagaaggactttgaaatctgggcaattaatgagcagaccatgaaactggaatccgcgaaagtgtcgcgcgtgttctgcactggtaagaagttggtctacatcctgaaaacccgtctgggccgtaccatcaaagcgaccgccaatcatcgttttctgacgattgatggttggaaacgtttggacgaactgtccctgaaagagcacattgcattgccgcgtaaacttgagagctctagcttacagctggccagcggccatcaccaccaccaccatggtggtagcggctcatccccggagatcgagaagctgtcgcaaagcgacatctattgggatagcatcgtgagcatcaccgaaaccggtgtagaggaagtattcgatctgaccgtcccgggtccgcataactttgttgcaaatgatattattgttcataac

SEQ ID NO: 115 - DNA, 507 bp, Artificial Sequence
  Codon-optimized sequence DnaB_v39 encoding Synechocystis sp. PCC6803 DnaB 6xHis-tagged non-ligating mini-intein (SEQ ID NO2)
atggcaatctctggcgatagccttatatcactagcgtccaccggtaagcgcgtgtccatcaaagacctgctggacgagaaggactttgaaatctgggcaattaatgagcagaccatgaaactggaatccgcgaaagtgtcgcgcgtgttctgcactggtaagaagttggtctacatcctgaaaacccgtctgggccgtaccatcaaagcgaccgccaatcatcgttttctgacgattgatggttggaaacgtttggacgaactgtccctgaaagagcacattgcattgccgcgtaaacttgagagctctagcttacagctggccagcggccatcaccaccaccaccatggtggtagcggctcatccccggagatcgagaagctgtcgcaaagcgacatctattgggatagcatcgtgagcatcaccgaaaccggtgtagaggaagtattcgatctgaccgtcccgggtccgcataactttgttgcaaatgatattattgttcataac

SEQ ID NO: 116 - DNA, 507 bp, Artificial Sequence
  Codon-optimized sequence DnaB_v40 encoding Synechocystis sp. PCC6803 DnaB 6xHis-tagged non-ligating mini-intein (SEQ ID NO2)
  misc_feature (6)..(6): n is a, c, g, or t
  misc_feature (12)..(12): n is a, c, g, or t
  misc_feature (15)..(15): n is a, c, g, or t
  misc_feature (21)..(21): n is a, c, g, or t
  misc_feature (24)..(24): n is a, c, g, or t
  misc_feature (33)..(33): n is a, c, g, or t
atggcnathtcnggngaytcnctnatyagyctngcttccacagggaagcgcgtatctattaaagatttgttggacgaaaaagattttgaaatttgggctatcaacgagcaaaccatgaagctggaatctgcaaaggtcagccgcgtcttctgcacaggtaaaaaacttgtttacattcttaaaacgcgtctgggtcgtacaatcaaggcgacagccaaccatcgctttttaaccattgatggttggaagcgtctggatgagttgtctctgaaggagcacatcgcgcttcctcgtaaactggagtcgtctagtttgcaattggcctccgggcaccatcaccatcatcatggggggtcggggtcttcgccggagatcgaaaagttgtcccagtccgacatctactgggattcgatcgttagcatcactgagacaggagtggaagaggtgttcgacttaacggtgcctggaccccataactttgtggcgaacgatatcatcgttcacaac

SEQ ID NO: 117 - DNA, 507 bp, Artificial Sequence
  Codon-optimized sequence DnaB_v41 encoding Synechocystis sp. PCC6803 DnaB 6xHis-tagged non-ligating mini-intein (SEQ ID NO2)
  misc_feature (6)..(6): n is a, c, g, or t
  misc_feature (12)..(12): n is a, c, g, or t
  misc_feature (15)..(15): n is a, c, g, or t
  misc_feature (21)..(21): n is a, c, g, or t
  misc_feature (24)..(24): n is a, c, g, or t
atggcnathtcnggngaytcnctnathagyttrgcttctaccggaaagcgcgtaagcatcaaagacctgcttgatgagaaagatttcgaaatctgggcgattaacgagcaaactatgaaattggagtctgctaaggtaagtcgcgtattctgcactgggaaaaagttagtttatattttgaaaacacgtttagggcgcactattaaggcaacagcaaatcaccgctttctgaccattgatggttggaaacgtctggatgagcttagtcttaaagaacacatcgctttgcctcgcaaacttgaatcgtcatcgcttcaactggcgtcaggccatcatcatcaccatcatgggggatcgggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 118 - DNA, 507 bp, Artificial Sequence
  Codon-optimized sequence DnaB_v42 encoding Synechocystis sp. PCC6803 DnaB 6xHis-tagged non-ligating mini-intein (SEQ ID NO2)
atggccatttctggtgattcccttatcagtctcgcttccacagggaagcgcgtatctattaaagatttgttggacgaaaaagattttgaaatttgggctatcaacgagcaaaccatgaagctggaatctgcaaaggtcagccgcgtcttctgcacaggtaaaaaacttgtttacattcttaaaacgcgtctgggtcgtacaatcaaggcgacagccaaccatcgctttttaaccattgatggttggaagcgtctggatgagttgtctctgaaggagcacatcgcgcttcctcgtaaactggagtcgtctagtttgcaattggcctccgggcaccatcaccatcatcatggggggtcggggtcttcgccggagatcgaaaagttgtcccagtccgacatctactgggattcgatcgttagcatcactgagacaggagtggaagaggtgttcgacttaacggtgcctggaccccataactttgtggcgaacgatatcatcgttcacaac

SEQ ID NO: 119 - DNA, 507 bp, Artificial Sequence
  Codon-optimized sequence DnaB_v43 encoding Synechocystis sp. PCC6803 DnaB 6xHis-tagged non-ligating mini-intein (SEQ ID NO2)
atggccatatctggcgattcccttattagtttagcttctaccggaaagcgcgtaagcatcaaagacctgcttgatgagaaagatttcgaaatctgggcgattaacgagcaaactatgaaattggagtctgctaaggtaagtcgcgtattctgcactgggaaaaagttagtttatattttgaaaacacgtttagggcgcactattaaggcaacagcaaatcaccgctttctgaccattgatggttggaaacgtctggatgagcttagtcttaaagaacacatcgctttgcctcgcaaacttgaatcgtcatcgcttcaactggcgtcaggccatcatcatcaccatcatgggggatcgggttcatctcccgagattgagaaactttcccagagcgacatctattgggatagtatcgtgagtattactgaaacgggggtggaggaggtgttcgacttgaccgtccctggtcctcataactttgttgcgaacgatattattgttcacaac

SEQ ID NO: 120 - protein, 169 aa, Artificial Sequence
  Synechocystis sp. PCC6803, DnaB; 4xHisKA-tagged mini-intein
MAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLASGHHHHKAGGSGSSPEIEKLSQSDIYWDSIVSITETGVEEVFDLTVPGPHNFVANDIIVHN

SEQ ID NO: 121 - protein, 169 aa, Artificial Sequence
  Synechocystis sp. PCC6803, DnaB; 4xHisKH-tagged mini-intein
MAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLASGHHHHKHGGSGSSPEIEKLSQSDIYWDSIVSITETGVEEVFDLTVPGPHNFVANDIIVHN

SEQ ID NO: 122 - protein, 169 aa, Artificial Sequence
  Synechocystis sp. PCC6803, DnaB; 4xHisKS-tagged mini-intein
MAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLASGHHHHKSGGSGSSPEIEKLSQSDIYWDSIVSITETGVEEVFDLTVPGPHNFVANDIIVHN

SEQ ID NO: 123 - protein, 169 aa, Artificial Sequence
  Synechocystis sp. PCC6803, DnaB; 4xHisKT-tagged mini-intein
MAISGDSLISLASTGKRVSIKDLLDEKDFEIWAINEQTMKLESAKVSRVFCTGKKLVYILKTRLGRTIKATANHRFLTIDGWKRLDELSLKEHIALPRKLESSSLQLASGHHHHKTGGSGSSPEIEKLSQSDIYWDSIVSITETGVEEVFDLTVPGPHNFVANDIIVHN

SEQ ID NO: 124 - protein, 155 aa, Artificial Sequence
  Synechocystis sp. PCC6803, DnaB; 6xHis-tagged mini-intein variant M86
MAISGDSLISLASTGKRVPIKDLLGEKDFEIWAINEQTMKLESAKVSRVFCTGKKLVYTLKTRLGRTIKATANHRFLTIDGWKRLDELSLKEHIALPRKLESSSLQLAPEIEKLPQSDIYWDPIVSITETGVEEVFDLTVPGLRNFVANDIIVHN